(19) United States
(12) Patent Application Publication
(10) Pub. No.: US 2004/0121338 A1
(43) Pub. Date: Jun. 24, 2004

(54) Real-time detection of rolling circle amplification products

(76) Inventors: Osama A. Alsmadi, Guilford, CT (US); Patricio Abarzua, West Caldwell, NJ (US)

Correspondence Address: NEEDLE & ROSENBERG, P.C., SUITE 1000, 999 PEACHTREE STREET, ATLANTA, GA, 30309-3915, US

(21) Appl. No.: 10/325,665

(22) Filed: Dec. 19, 2002

(51) Int. Cl. C12Q 1/70; C12Q 1/68; C12P 19/34

(52) U.S. Cl. 435/6; 435/91.2

(57) ABSTRACT

Disclosed are compositions and methods for real-time detection of rolling circle amplification products. Real-time detection is detection that takes place during the amplification reaction or operation. Real-time detection can be accomplished by, for example, using fluorescent change probes and/or primers during amplification. The fluorescent signals can be proportional to the amount of amplification product. The amplification can be multiply-primed rolling circle amplification in which replication of a circular template is primed at a plurality of sites on the circular template. Multiply-primed RCA increases the sensitivity of singly-primed rolling circle amplification. Multiply-primed RCA can be performed using a single primer (which hybridizes to multiple sites on the amplification target circle) or multiple primers (each of which can hybridize to a single site on the amplification target circle or multiple sites on the amplification target circle). Fluorescent change probes and primers are probes and primers that involve a change in fluorescence intensity or wavelength based on a change in the form or conformation of the probe or primer and nucleic acid to be detected, assayed or replicated.

### FIELD OF THE INVENTION

The disclosed invention is generally in the field of nucleic amplification and detection and specifically in the area of detection of rolling circle amplification products during amplification.

### BACKGROUND OF THE INVENTION

Numerous nucleic acid amplification techniques have been devised, including strand displacement cascade amplification (SDCA)(referred to herein as exponential rolling circle amplification (ERCA)) and rolling circle amplification (RCA)(U.S. Pat. No. 5,854,033; PCT Application No. WO 97/19193; Lizardi et al., Nature Genetics 19(3):225-232 (1998)); multiple displacement amplification (MDA)(PCT Application WO 99/18241); strand displacement amplification (SDA)(Walker et al., Nucleic Acids Research 20:1691-1696 (1992), Walker et al., Proc. Natl. Acad. Sci. USA 89:392-396 (1992)); polymerase chain reaction (PCR) and other exponential amplification techniques involving thermal cycling, self-sustained sequence replication (3SR), nucleic acid sequence based amplification (NASBA), and amplification with Qβ replicase (Birkenmeyer and Mushahwar, J Virological Methods 35:117-126 (1991); Landegren, Trends Genetics 9:199-202 (1993)); and various linear amplification techniques involving thermal cycling such as cycle sequencing (Craxton et al., Methods Companion Methods in Enzymology 3:20-26 (1991)).

Rolling Circle Amplification (RCA) driven by DNA polymerase can replicate circular oligonucleotide probes with either linear or geometric kinetics under isothermal conditions (Lizardi et al., Nature Genet. 19: 225-232 (1998); U.S. Pat. Nos. 5,854,033 and 6,143,495; PCT Application No. WO 97/19193). If a single primer is used, RCA generates in a few minutes a linear chain of hundreds or thousands of tandemly-linked DNA copies of a target that is covalently linked to that target. Generation of a linear amplification product permits both spatial resolution and accurate quantitation of a target. DNA generated by RCA can be labeled with fluorescent oligonucleotide tags that hybridize at multiple sites in the tandem DNA sequences. RCA can be used with fluorophore combinations designed for multiparametric color coding (PCT Application No. WO 97/19193), thereby markedly increasing the number of targets that can be analyzed simultaneously. RCA technologies can be used in solution, in situ and in microarrays. In solid phase formats, detection and quantitation can be achieved at the level of single molecules (Lizardi et al., 1998). Ligation-mediated Rolling Circle Amplification (LM-RCA) involves circularization of a probe molecule hybridized to a target sequence and subsequent rolling circle amplification of the circular probe (U.S. Pat. Nos. 5,854,033 and 6,143,495; PCT Application No. WO 97/19193). Very high yields of amplified products can be obtained with exponential rolling circle amplification (U.S. Pat. Nos. 5,854,033 and 6,143,495; PCT Application No. WO 97/19193) and multiply-primed rolling circle amplification (Dean et al., Genome Research 11:1095-1099 (2001)).

### BRIEF SUMMARY OF THE INVENTION

Disclosed are compositions and methods for real-time detection of rolling circle amplification products. Real-time detection is detection that takes place during the amplification reaction or operation. Generally, such detection can be accomplished by detecting amplification product at one or more discrete times during amplification, continuously during all or one or more portions of the amplification, or a combination of discrete times and continuous detection. Real-time detection can be aided by the use of labels or moieties that embody or produce a detectable signal that can be detected without disrupting the amplification reaction or operation. Fluorescent labels are an example of useful labels for real-time detection. A particularly useful means of obtaining real-time detection is the use of fluorescent change probes and/or primers in the amplification operation. With suitably designed fluorescent change probes and primers, fluorescent signals can be generated as amplification proceeds. In most such cases, the fluorescent signals will be in proportion to the amount of amplification product and/or amount of target sequence or target molecule.

In some forms, the disclosed method involves rolling circle amplification and real-time detection of amplification products where amplification includes multiply-primed rolling circle amplification (MPRCA). Rolling circle amplification (RCA) refers to nucleic acid amplification reactions involving replication of a circular nucleic acid template (referred to as an amplification target circle; ATC) to form a long strand (referred to as tandem sequence DNA; TS-DNA) with tandem repeats of the sequence complementary to the circular template. Rolling circle replication can be primed at one or more sites on the circular template. Multiply-primed RCA refers to RCA where replication is primed at a plurality of sites on the circular template. Multiply-primed RCA increases the sensitivity of singly-primed rolling circle amplification. Rolling circle amplification refers both to rolling circle replication and to processes involving both rolling circle replication and additional forms of amplification (such as replication of tandem sequence DNA).

Multiply-primed RCA can be performed using a single primer (which hybridizes to multiple sites on the amplification target circle) or multiple primers (each of which can hybridize to a single site on the amplification target circle or multiple sites on the amplification target circle). Multiple priming (as occurs in MPRCA) can increase the yield of amplified product from RCA. Primers anneal to multiple locations on the circular template and a product of extension by polymerase is initiated from each location. In this way, multiple extensions are achieved simultaneously from a single amplification target circle.

In some forms of the disclosed method, multiple priming can be achieved in several different ways. For example, two or more specific primers that anneal to different sequences on the circular template can be used, one or more specific primers that each anneals to a sequence repeated at two or more separate locations on the circular template can be used, a combination of primers that each anneal to a different sequence on the circular template or to a sequence repeated at two or more separate locations on the circular templates can be used, one or more random or degenerate primers, which can anneal to many locations on the circle, can be used, or a combination of such primers can be used.

Fluorescent change probes and primers, which are useful for obtaining real-time detection of amplification, refer to all probes and primers that involve a change in fluorescence intensity or wavelength based on a change in the form or conformation of the probe or primer and nucleic acid to be detected, assayed or replicated. Examples of fluorescent change probes and primers include molecular beacons, Amplifluors, FRET probes, cleavable FRET probes, TaqMan probes, scorpion primers, fluorescent triplex oligos, including but not limited to triplex molecular beacons or triplex FRET probes, fluorescent water-soluble conjugated polymers, PNA probes and QPNA probes. Change in fluorescence wavelength or intensity from fluorescent change probes and primers generally involves energy transfer and/or quenching. Fluorescent change probes and primers can be classified according to their structure and/or function. Fluorescent change probes include, for example, hairpin quenched probes, cleavage quenched probes, cleavage activated probes, and fluorescent activated probes.

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A, 1B and 1C are diagrams illustrating multiply-primed rolling circle amplification. FIG. 1A shows multiple priming of rolling circle replication on the same circular template. FIG. 1B shows strand displacement of multiple growing strands (TS-DNA) on the same circular template. FIG. 1C shows strand displacement replication of the TS-DNA.

FIG. 2 is a diagram illustrating an example of the disclosed method showing multiply-primed rolling circle amplification using molecular beacons to allow real-time detection of amplification during the amplification reaction.

FIGS. 3A, 3B, 3C and 3D are diagrams of four fluorescent change probes directed to sequences in the ampicillin-resistance gene of pUC19. The fluorescent change probes shown are AMP-MB1 (FIG. 3A; SEQ ID NO:1), AMP-MB2 (FIG. 3B; SEQ ID NO:2), AMP-MB3 (FIG. 3C; SEQ ID NO:3) and AMP-MB4 (FIG. 3D; SEQ ID NO:4)

FIG. 4 is graph of incubation time (in minutes) versus fluorescence for examples of multiply-primed RCA using fluorescent change probes for real-time detection of amplification. The different curves were generated form different reactions each containing a different amount of circular template (that is, plasmid pUC19).

FIG. 5 is a graph of the log of the number of copies of circular template (pUC19) versus the time (in minutes) at which fluorescence over background was first detected in the multiply-primed RCA reactions depicted in FIG. 4.

FIG. 6 is a graph of incubation time (in minutes) versus fluorescence for examples of multiply-primed RCA using fluorescent change probes for real-time detection of amplification. The different curves were generated form different reactions each containing a different cell lysate from cells harboring different plasmids (pUC19, pNEB, pBR322, pUC19-HCV) plus a no plasmid control lysate (E. coli).

### DETAILED DESCRIPTION OF THE INVENTION

The disclosed method and compositions can be understood more readily by reference to the following detailed description of particular embodiments and the Example included therein and to the Figures and their previous and following description.

Disclosed are compositions and methods for real-time detection of rolling circle amplification products. Real-time detection is detection that takes place during the amplification reaction or operation. Generally, such detection can be accomplished by detecting amplification product at one or more discrete times during amplification, continuously during all or one or more portions of the amplification, or a combination of discrete times and continuous detection. Real-time detection can be aided by the use of labels or moieties that embody or produce a detectable signal that can be detected without disrupting the amplification reaction or operation. Fluorescent labels are an example of useful labels for real-time detection. A particularly useful means of obtaining real-time detection is the use of fluorescent change probes and/or primers in the amplification operation. With suitably designed fluorescent change probes and primers, fluorescent signals can be generated as amplification proceeds. In most such cases, the fluorescent signals will be in proportion to the amount of amplification product and/or amount of target sequence or target molecule.

In some forms, the disclosed method involves rolling circle amplification and real-time detection of amplification products where amplification includes multiply-primed rolling circle amplification (MPRCA). Rolling circle amplification (RCA) refers to nucleic acid amplification reactions involving replication of a circular nucleic acid template (referred to as an amplification target circle; ATC) to form a long strand (referred to as tandem sequence DNA; TS-DNA) with tandem repeats of the sequence complementary to the circular template. Rolling circle replication can be primed at one or more sites on the circular template. Multiply-primed RCA refers to RCA where replication is primed at a plurality of sites on the circular template. Multiply-primed RCA increases the sensitivity of singly-primed rolling circle amplification. Rolling circle amplification refers both to rolling circle replication and to processes involving both rolling circle replication and additional forms of amplification (such as replication of tandem sequence DNA).

Multiply-primed RCA can be performed using a single primer (which hybridizes to multiple sites on the amplification target circle) or multiple primers (each of which can hybridize to a single site on the amplification target circle or multiple sites on the amplification target circle). Multiple priming (as occurs in MPRCA) can increase the yield of amplified product from RCA. Primers anneal to multiple locations on the circular template and a product of extension by polymerase is initiated from each location. In this way, multiple extensions are achieved simultaneously from a single amplification target circle.

In some forms of the disclosed method, multiple priming can be achieved in several different ways. For example, two or more specific primers that anneal to different sequences on the circular template can be used, one or more specific primers that each anneals to a sequence repeated at two or more separate locations on the circular template can be used, a combination of primers that each anneal to a different sequence on the circular template or to a sequence repeated at two or more separate locations on the circular templates can be used, one or more random or degenerate primers, which can anneal to many locations on the circle, can be used, or a combination of such primers can be used.

Multiply-primed rolling circle amplification generates multiple tandem-sequence DNA (TS-DNA) copies from each circular template molecule. MPRCA can be used with circular template molecules of known, partially known, or unknown sequence, and the circular target DNA molecule can be single-stranded (ssDNA), double-stranded (dsDNA or duplex DNA), or partially double-stranded. Random or degenerate primers are useful for RCA of circular templates of unknown sequence.

Any or all of the primers used in the disclosed method can be resistant to degradation by exonuclease activity that may be present in the reaction. This has the advantage of permitting the primers to persist in reactions that contain an exonuclease activity and that may be carried out for long incubation periods. The persistence of primers allows new priming events to occur for the entire incubation time of the reaction, which is one of the hallmarks of exponential RCA (ERCA) and has the advantage of increasing the yield of amplified DNA.

Fluorescent change probes and primers, which are useful for obtaining real-time detection of amplification, refer to all probes and primers that involve a change in fluorescence intensity or wavelength based on a change in the form or conformation of the probe or primer and nucleic acid to be detected, assayed or replicated. Examples of fluorescent change probes and primers include molecular beacons, Amplifluors, FRET probes, cleavable FRET probes, TaqMan probes, scorpion primers, fluorescent triplex oligos including but not limited to triplex molecular beacons or triplex FRET probes, fluorescent water-soluble conjugated polymers, PNA probes and QPNA probes. Change in fluorescence wavelength or intensity from fluorescent change probes and primers generally involves energy transfer and/or quenching. Fluorescent change probes and primers can be classified according to their structure and/or function. Fluorescent change probes include, for example, hairpin quenched probes, cleavage quenched probes, cleavage activated probes, and fluorescent activated probes.

Random and/or degenerate probes and primers can be used with the disclosed method. As used herein, degenerate refers to an oligonucleotide (or oligomer) in which one or more of the base positions is occupied by more than one base, that is, a mixture of oligonucleotides (or oligomers) of defined length in which one or more positions of an individual member of the mixture is occupied by a base selected at random from among more than one possibilities for that position. Such collections of oligonucleotides (or oligomers) can be readily synthesized using standard oligonucleotide synthesis instruments and software. As used herein, random refers to an oligonucleotide (or oligomer) in which each of the base positions is occupied by a base selected at random from among a complete set of possibilities, but commonly limited to, for example, the four bases adenine (A), guanine (G), cytosine (C) and thymine (T) (or uracil (U)). For example, random oligonucleotides can be composed of the four nucleotides deoxyriboadenosine monophosphate (dAMP), deoxyribocytidine monophosphate (dCMP), deoxyriboguanosine monophosphate (dGMP), or deoxyribothymidine monophosphate (dTMP). Degenerate oligonucleotides (or oligomers) where not every base position is selected at random from among a complete set of possibilities can be referred to as partially random oligonucleotides (or oligomers). In some embodiments, the primers can contain nucleotides, including any types of modified nucleotides or nucleotide analogs, which can serve to make the primers resistant to enzyme degradation, to have other effects, or to give the primers useful properties.

The disclosed method can be used to amplify and detect any circular molecule. Circular template molecules to be subject to rolling circle replication and rolling circle amplification are referred to herein as amplification target circles (ATC). Amplification target circles can be, for example, designed and prefabricated for use in the disclosed method or can be produced from nucleic acid sources and samples of interest. For example, in some forms of the disclosed method, amplification target circles are designed and synthesized to have specific features making them useful for particular forms of the disclosed method. Such features are described in detail elsewhere herein. Amplification target circles can be circularized open circle probes. Such circularization is usefully accomplished via target-mediated ligation of the ends of the open circle probe. Amplification target circles can also be produced by circularizing nucleic acid molecules of interest or inserting nucleic acid molecules of interest into, for example, linker, vector or circularization sequences. Thus, target sequences can be copied or inserted into circular ssDNA or dsDNA by any suitable cloning or recombinant DNA technique. Amplification target circles can also be circular nucleic acid molecules isolated from cell, tissues or other nucleic acid samples. For example, plasmid DNA, viral DNA, and other circular nucleic acids can be used as amplification target circles in the disclosed method.

Genomic sequences can be amplified using the disclosed method. For example, known sequences or sequences of interest from genomic or other complex DNAs can be circularized or otherwise placed in amplification target circles for use in the disclosed method. Alternatively, amplification target circles generated in or from a whole genome amplification method can be used in the disclosed method. Whole genome amplification can involve randomly primed or specifically primed generation of all or a subset of genomic, cDNA or other complex DNA. Any suitable method then can be used to circularize the products of the whole genome amplification. The resulting amplification target circles could then be amplified in the disclosed method. Regardless of the means used to generate the circular products of whole genome amplification, multiply-primed RCA (using random primers, for example) would allow the selective amplification of the circles over any background of linear DNAs without the need for knowing the sequence of the circles. Alternatively, circular DNA containing known linker, vector, circularizing or target sequences would allow use of specific primer sequences for multiply-primed RCA.

Multiply-primed RCA represents an improvement over linear RCA (LRCA) in allowing increased rate of synthesis and increased yield. This results from the multiple priming sites for DNA polymerase extension. Use of random or degenerate primers also can have the benefit of generating double stranded products. This is because the linear ssDNA products generated by copying of the circular template will themselves be converted to duplex form by random (or degenerate) priming of DNA synthesis. Double stranded products can also be generated in most forms of DNA strand displacement replication, such as exponential RCA. Double stranded DNA product is advantageous in allowing for DNA sequencing of either strand and for restriction endonuclease digestion and other methods used in cloning, labeling, and detection.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

### Materials

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a rolling circle replication primer is disclosed and discussed and a number of modifications that can be made to a number of molecules including the rolling circle replication primer are discussed, each and every combination and permutation of the rolling circle replication primer and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

A. Amplification Target Circles

An amplification target circle (ATC) is a circular DNA molecule. ATCs are preferably single-stranded but can be partially or fully double-stranded. Portions of ATCs have specific functions making the ATC useful for rolling circle amplification (RCA). These portions are referred to as the primer complement portions, the secondary DNA strand displacement primer matching portions, the detection tag portions, the secondary target sequence portions, the address tag portions, and the promoter portions. At least one primer complement portion is a required element of an amplification target circle. For multiply-primed RCA, a plurality of primer complement portions are required. Where random or degenerate rolling circle replication primers are used, the sequence of the primer complement portions need not either be known or of a specified sequence. The amplification target circle can include at least one detection tag portion when fluorescent change probes (or other detection probes) are used for detection.

Secondary DNA strand displacement primer matching portions, detection tag portions, secondary target sequence portions, address tag portions, and promoter portions are optional. The primer complement portions, the secondary DNA strand displacement primer matching portions, the detection tag portions, the secondary target sequence portions, the address tag portions, and the promoter portion, if present, are preferably non-overlapping. However, various of these portions can be partially or completely overlapping if desired. Generally, an amplification target circle can be a circular DNA molecule comprising one or more primer complement portions. Amplification target circles can be single-stranded, double-stranded, or partially double-stranded. Useful amplification target circles can comprise one or more primer complement portions, one or more secondary DNA strand displacement primer matching portions, and one or more detection tag portions.

Those segments of the ATC that do not correspond to a specific portion of the ATC can be arbitrarily chosen sequences. It is preferred that ATCs do not have any sequences that are self-complementary, although this is required. It is considered that this condition is met if there are no complementary regions greater than six nucleotides long without a mismatch or gap. It is also preferred that ATCs containing a promoter portion do not have any sequences that resemble a transcription terminator, such as a run of eight or more thymidine nucleotides. A lack of self-complementary sequences and a lack of promoter sequences is generally not required in the case of amplification target circles including, derived from, or comprising nucleic acid molecules of interest. such features will generally not be controlled for such amplification target circles.

Ligated and circularized open circle probes are a type of ATC, and as used herein the term amplification target circle includes ligated open circle probes and circularized open circle probes. An ATC can be used in the same manner as described herein for OCPs that have been ligated or circularized. Amplification target circles can be any desired length. Generally, amplification target circles designed for use as amplifiable labels can contain between 40 to 1000 nucleotides, more preferably between about 50 to 150 nucleotides, and most preferably between about 50 to 100 nucleotides. Amplification target circles including, derived from, or comprising nucleic acid molecules of interest can be any useful size, including, for example, the size of a plasmid, virus, vector, or artificial chromosome.

An amplification target circle, when replicated, gives rise to a long DNA molecule containing multiple repeats of sequences complementary to the amplification target circle. This long DNA molecule is referred to herein as tandem sequences DNA (TS-DNA). TS-DNA contains sequences complementary to the primer complement portions and, if present on the amplification target circle, the secondary DNA strand displacement primer matching portions, the detection tag portions, the secondary target sequence portions, the address tag portions, and the promoter portion. These sequences in the TS-DNA are referred to as primer sequences (which match the sequence of the rolling circle replication primers), spacer sequences (complementary to the spacer region), detection tags, secondary target sequences, address tags, and promoter sequences. The TS-DNA will also have sequence complementary to the matching portion of secondary DNA strand displacement primers. This sequence in the TS-DNA is referred to as the secondary DNA strand displacement primer complement or as the primer complement. Amplification target circles are useful as tags for specific binding molecules.

1. Primer Complement Portions

Primer complement portions are parts of an amplification target circle that are complementary to rolling circle replication primers (RCRP). Each ATC preferably has multiple primer complement portions. This allows rolling circle replication to initiate at multiple sites on the ATC. However, an ATC can include one or more than one primer complement portion. If multiple primer complement portions are present, they can have sequence complementary to the same rolling circle replication primer, different rolling circle replication primers, or a combination of the same and different rolling circle replication primers. A primer complement portion and its cognate primer can have any desired sequence so long as they are complementary to each other. The sequence of the primer complement portion is referred to as the primer complement sequence.

In general, the sequence of a primer complement can be chosen such that it is not significantly similar to any other portion of the ATC. The primer complement portion can be any length that supports specific and stable hybridization between the primer complement portion and the primer. For this purpose, a length of 10 to 35 nucleotides is preferred, with a primer complement portion 16 to 20 nucleotides long being most preferred. If random or degenerate rolling circle replication primers are used, the amplification target circles will have multiple primer complement portions that generally will not be, and need not be, specifically identified. If random or degenerate rolling circle replication primers are used, the primers and the primer complement portions are preferably 4 to 10 nucleotides long, and most preferably 6, 7 or 8 nucleotides long.

The primer complement portions can be located anywhere on the ATC, such as within the spacer region of an ATC. Primer complement portions can be anywhere on the ATC or circularized OCP. For example, the primer complement portions can be adjacent to the right target probe, with the right target probe portion and the primer complement portion preferably separated by three to ten nucleotides, and most preferably separated by six nucleotides, from the proximate primer complement portion. This location prevents the generation of any other spacer sequences, such as detection tags and secondary target sequences, from unligated open circle probes during DNA replication. Such an arrangement is less useful when using multiply-primed RCA. A primer complement portion can also be a part of or overlap all or a part of the target probe portions and/or any gap space sequence, if present.

2. Secondary DNA Strand Displacement Primer Matching Portions

Secondary DNA strand displacement primer matching portions are parts of an amplification target circle that match sequence in secondary DNA strand displacement primers. The sequence in a secondary DNA strand displacement primer that matches a secondary DNA strand displacement primer matching portion in an ATC is referred to as the matching portion of the secondary DNA strand displacement primer. An ATC can include one or more than one primer matching portion. If multiple primer matching portions are present, they can have sequence matching the same secondary DNA strand displacement primer (which is preferred), different secondary DNA strand displacement primers, or a combination of the same and different secondary DNA strand displacement primers. A single secondary DNA strand displacement primer matching portion is preferred. A primer matching portion and its cognate primer can have any desired sequence so long as they are complementary to each other. The sequence of the primer matching portion can be referred to as the primer matching sequence. More specifically, the sequence of the secondary DNA strand displacement primer matching portion can be referred to as the secondary DNA strand displacement primer matching sequence.

In general, the sequence of a primer matching portion can be chosen such that it is not significantly similar to any other portion of the ATC. Primer matching portions can overlap with primer complement portions, although it is preferred that they not overlap. The primer matching portion can be any length that supports specific and stable hybridization between the primer complement portion in the resulting TS-DNA and the primer. For this purpose, a length of 10 to 35 nucleotides is preferred, with a primer matching portion 16 to 20 nucleotides long being most preferred. The primer matching portion can be located anywhere on the ATC, such as within the spacer region of an ATC. Primer matching portions can be anywhere on the ATC or circularized OCP. If random or degenerate rolling circle replication primers are used, they can act as secondary DNA strand displacement primer. In this case, the amplification target circles will have multiple secondary DNA strand displacement primer matching portions that generally will not be, and need not be, specifically identified. If random or degenerate rolling circle replication primers are used, the primers and the secondary DNA strand displacement primer matching portions are preferably 4 to 10 nucleotides long, and most preferably 6, 7 or 8 nucleotides long.

3. Detection Tag Portions

Detection tag portions are part of the spacer region of an amplification target circle. Detection tag portions have sequences matching the sequence of the complementary portion of detection probes. These detection tag portions, when amplified during rolling circle replication, result in TS-DNA having detection tag sequences that are complementary to the complementary portion of detection probes. If present, there can be one, two, three, or more than three detection tag portions on an ATC. For example, an ATC can have two, three or four detection tag portions. Most preferably, an ATC will have three detection tag portions. Generally, it is preferred that an ATC have 60 detection tag portions or less. There is no fundamental limit to the number of detection tag portions that can be present on an ATC except the size of the ATC. When there are multiple detection tag portions, they can have the same sequence or they can have different sequences, with each different sequence complementary to a different detection probe. It is preferred that an ATC contain detection tag portions that have the same sequence such that they are all complementary to a single detection probe. For some multiplex detection methods, it is preferable that ATCs contain up to six detection tag portions and that the detection tag portions have different sequences such that each of the detection tag portions is complementary to a different detection probe. If the amplification target circles include, are derived from, or comprise nucleic acid molecules of interest, some or all of the detection tag portions can be sequences of interest in the nucleic acid of interest. In this way, detection can be based on the amplification of the specific sequences of interest. The detection tag portions can each be any length that supports specific and stable hybridization between the detection tags and the detection probe. For this purpose, a length of 10 to 35 nucleotides is preferred, with a detection tag portion 15 to 20 nucleotides long being most preferred.

4. Secondary Target Sequence Portions

Secondary target sequence portions are part of the spacer region of an amplification target circle. Secondary target sequence portions have sequences matching the sequence of target probes of a secondary open circle probe. These secondary target sequence portions, when amplified during rolling circle replication, result in TS-DNA having secondary target sequences that are complementary to target probes of a secondary open circle probe. If present, there can be one, two, or more than two secondary target sequence portions on an ATC. It is preferred that an ATC have one or two secondary target sequence portions. Most preferably, an ATC will have one secondary target sequence portion. Generally, it is preferred that an ATC have 50 secondary target sequence portions or less. There is no fundamental limit to the number of secondary target sequence portions that can be present on an ATC except the size of the ATC. When there are multiple secondary target sequence portions, they can have the same sequence or they can have different sequences, with each different sequence complementary to a different secondary OCP. It is preferred that an ATC contain secondary target sequence portions that have the same sequence such that they are all complementary to a single target probe portion of a secondary OCP. If the amplification target circles include, are derived from, or comprise nucleic acid molecules of interest, some or all of the secondary target sequence portions can be sequences of interest in the nucleic acid of interest. In this way, further amplification can be based on the presence of the specific sequences of interest.

The secondary target sequence portions can each be any length that supports specific and stable hybridization between the secondary target sequence and the target sequence probes of its cognate secondary OCP. For this purpose, a length of 20 to 70 nucleotides is preferred, with a secondary target sequence portion 30 to 40 nucleotides long being most preferred. As used herein, a secondary open circle probe is an open circle probe where the target probe portions match or are complementary to secondary target sequences in another open circle probe or an amplification target circle. It is contemplated that a secondary open circle probe can itself contain secondary target sequences that match or are complementary to the target probe portions of another secondary open circle probe. Secondary open circle probes related to each other in this manner are referred to herein as nested open circle probes.

5. Address Tag Portions

The address tag portion is part of an amplification target circle. The address tag portion has a sequence matching the sequence of the complementary portion of an address probe. This address tag portion, when amplified during rolling circle replication, results in TS-DNA having address tag sequences that are complementary to the complementary portion of address probes. If present, there can be one, or more than one, address tag portions on an ATC. It is preferred that an ATC have one or two address tag portions. Most preferably, an ATC will have one address tag portion. Generally, it is preferred that an ATC have 50 address tag portions or less. There is no fundamental limit to the number of address tag portions that can be present on an ATC except the size of the ATC. When there are multiple address tag portions, they can have the same sequence or they can have different sequences, with each different sequence complementary to a different address probe. It is preferred that an ATC contain address tag portions that have the same sequence such that they are all complementary to a single address probe. The address tag portion can be any length that supports specific and stable hybridization between the address tag and the address probe. For this purpose, a length between 10 and 35 nucleotides long is preferred, with an address tag portion 15 to 20 nucleotides long being most preferred. Where the ATC is formed from an OCP, the address tag portion can be part either the target probe portions or the spacer region. In this case, the address tag portion preferably overlaps all or a portion of the target probe portions, and all of any intervening gap space. Most preferably, the address tag portion overlaps all or a portion of both the left and right target probe portions.

6. Promoter Portions

The promoter portion corresponds to the sequence of an RNA polymerase promoter. A promoter portion can be included in an amplification target circle so that transcripts can be generated from the ATC or TS-DNA. The sequence of any promoter can be used, but simple promoters for RNA polymerases without complex requirements are preferred. It is also preferred that the promoter is not recognized by any RNA polymerase that may be present in the sample containing the target nucleic acid sequence. Preferably, the promoter portion corresponds to the sequence of a T7 or SP6 RNA polymerase promoter. The T7 and SP6 RNA polymerases are highly specific for particular promoter sequences. Other promoter sequences specific for RNA polymerases with this characteristic would also be preferred. Because promoter sequences are generally recognized by specific RNA polymerases, the cognate polymerase for the promoter portion of the ATC should be used for transcriptional amplification. Numerous promoter sequences are known and any promoter specific for a suitable RNA polymerase can be used. The promoter portion can be located anywhere within the spacer region of an ATC and can be in either orientation.

B. Rolling Circle Replication Primers

A rolling circle replication primer (RCRP) is an oligonucleotide or oligomer having sequence complementary to one or more primer complement portions of an OCP or ATC. This sequence is referred to as the complementary portion of the RCRP. The complementary portion of a RCRP and the cognate primer complement portion can have any desired sequence so long as they are complementary to each other. In general, the sequence of the RCRP can be chosen such that it is not significantly complementary to any other portion of the OCP or ATC. That is, the RCRP would be complementary only to primer complement portions. If random or degenerate rolling circle replication primers are used, the primers collectively will be complementary to many sequences on an ATC or OCP. The complementary portion of a rolling circle replication primer can be any length that supports specific and stable hybridization between the primer and the primer complement portion. Generally this is 10 to 35 nucleotides long, but is preferably 16 to 20 nucleotides long. Random or degenerate rolling circle replication primers are preferably 4 to 10 nucleotides long, and most preferably 6, 7 or 8 nucleotides long. Useful rolling circle replication primers are fluorescent change primers.

It is preferred that rolling circle replication primers also contain additional sequence at the 5′ end of the RCRP that is not complementary to any part of the OCP or ATC. This sequence is referred to as the non-complementary portion of the RCRP. The non-complementary portion of the RCRP, if present, can serve to facilitate strand displacement during DNA replication. The non-complementary portion of a RCRP can be any length, but is generally 1 to 100 nucleotides long, and preferably 4 to 8 nucleotides long. The non-complementary portion can be involved in interactions that provide specialized effects. For example, the non-complementary portion can comprise a quencher complement portion that can hybridize to a peptide nucleic acid quencher or peptide nucleic acid fluor or that can form an intramolecular structure. Random or degenerate rolling circle replication primers preferably do not include a non-complementary portion. Rolling circle replication primers can also comprise fluorescent moieties or labels and quenching moieties. Rolling circle replication primers can be capable of forming an intramolecular stem structure involving one or both of the RCRP's ends. Such rolling circle replication primers are referred to herein as hairpin rolling circle replication primers. Primers forming intramolecular stem structures, and their use in rolling circle amplification, are described in U.S. patent application Ser. No. 09/803,713.

Rolling circle replication primers can also include modified nucleotides to make it resistant to exonuclease digestion. For example, the primer can have three or four phosphorothioate linkages between nucleotides at the 5′ end of the primer. Such nuclease resistant primers allow selective degradation of excess unligated OCP and gap oligonucleotides that might otherwise interfere with hybridization of detection probes, address probes, and secondary OCPs to the amplified nucleic acid. A rolling circle replication primer can be used as the tertiary DNA strand displacement primer in strand displacement cascade amplification. Random or degenerate rolling circle replication primers can serve as secondary and tertiary DNA strand displacement primers.

A rolling circle replication primer is specific for, or corresponds to, an open circle probe or amplification target circle when the complementary portion of the rolling circle replication primer is complementary to the primer complement portion of the open circle probe or amplification target circle. A rolling circle replication primer is not specific for, or does not correspond to, an open circle probe or amplification target circle when the complementary portion of the rolling circle replication primer is not substantially complementary to the open circle probe or amplification target circle. A complementary portion is not substantially complementary to another sequence if it has a melting temperature 10° C. lower than the melting temperature under the same conditions of a sequence fully complementary to the complementary portion of the rolling circle replication primer.

A rolling circle replication primer is specific for, or corresponds to, a set of open circle probes or a set of amplification target circles when the complementary portion of the rolling circle replication primer is complementary to the primer complement portion of the open circle probes or amplification target circles in the set. A rolling circle replication primer is not specific for, or does not correspond to, a set of open circle probes or a set of amplification target circles when the complementary portion of the rolling circle replication primer is not substantially complementary to the open circle probes or amplification target circles in the set.

C. DNA Strand Displacement Primers

Primers used for secondary DNA strand displacement are referred to herein as DNA strand displacement primers. One form of DNA strand displacement primer, referred to herein as a secondary DNA strand displacement primer, is an oligonucleotide or oligomer having sequence matching part of the sequence of an OCP or ATC. This sequence in the secondary DNA strand displacement primer is referred to as the matching portion of the secondary DNA strand displacement primer. The sequence in the OCP or ATC that matches the matching portion of the secondary DNA strand displacement primer is referred to as the secondary DNA strand displacement primer matching portion. The matching portion of a secondary DNA strand displacement primer is complementary to sequences in TS-DNA. The matching portion of a secondary DNA strand displacement primer may be complementary to any sequence in TS-DNA. However, it is preferred that it not be complementary TS-DNA sequence matching either the rolling circle replication primers or a tertiary DNA strand displacement primer, if one is being used. This prevents hybridization of the primers to each other.

The matching portion of a secondary DNA strand displacement primer may be complementary to all or a portion of the target sequence. In this case, it is preferred that the 3′ end nucleotides of the secondary DNA strand displacement primer are complementary to the gap sequence in the target sequence. It is most preferred that nucleotide at the 3′ end of the secondary DNA strand displacement primer falls complementary to the last nucleotide in the gap sequence of the target sequence, that is, the 5′ nucleotide in the gap sequence of the target sequence. The matching portion of a secondary DNA strand displacement primer can be any length that supports specific and stable hybridization between the primer and its complement. Generally this is 12 to 35 nucleotides long, but is preferably 18 to 25 nucleotides long.

Secondary DNA strand displacement primers can be specific for, or correspond to, all of the open circle probes or amplification target circles in an amplification reaction or in a set of open circle probes or set of amplification target circles in an amplification reaction. A secondary DNA strand displacement primer is specific for, or corresponds to, an open circle probe or amplification target circle when the matching portion of the secondary DNA strand displacement primer matches the primer complement portion of the open circle probe or amplification target circle. A secondary DNA strand displacement primer is not specific for, or does not correspond to, an open circle probe or amplification target circle when the matching portion of the secondary DNA strand displacement primer does not substantially match sequence in the open circle probe or amplification target circle. A matching portion does not substantially match another sequence if it has a melting temperature with the complement of the other sequence that is 10° C. lower than the melting temperature under the same conditions of a sequence fully complementary to the matching portion of the secondary DNA strand displacement primer.

A secondary DNA strand displacement primer is specific for, or corresponds to, a set of open circle probes or a set of amplification target circles when the matching portion of the secondary DNA strand displacement primer matches the primer complement portion of the open circle probes or amplification target circles in the set. A secondary DNA strand displacement primer is not specific for, or does not correspond to, a set of open circle probes or a set of amplification target circles when the matching portion of the secondary DNA strand displacement primer does not substantially match the open circle probes or amplification target circles in the set. Secondary DNA strand displacement primers can be fluorescent change primers although this is not preferred.

It is preferred that secondary DNA strand displacement primers also contain additional sequence at the 5′ end of the primer that does not match any part of the OCP or ATC. This sequence is referred to as the non-matching portion of the secondary DNA strand displacement primer. The non-matching portion of the secondary DNA strand displacement primer, if present, can serve to facilitate strand displacement during DNA replication. The non-matching portion of a secondary DNA strand displacement primer may be any length, but is generally 1 to 100 nucleotides long, and preferably 4 to 8 nucleotides long. The non-matching portion can be involved in interactions that provide specialized effects. For example, the non-matching portion can comprise a quencher complement portion that can hybridize to a peptide nucleic acid quencher or peptide nucleic acid fluor or that can form an intramolecular structure. Secondary DNA strand displacement primers can also comprise fluorescent moieties or labels and quenching moieties.

Useful secondary DNA strand displacement primers for use in the disclosed method can form an intramolecular stem structure involving one or both of the secondary DNA strand displacement primer's ends. Such secondary DNA strand displacement primers are referred to herein as hairpin secondary DNA strand displacement primers. Primers forming intramolecular stem structures, and their use in rolling circle amplification, are described in U.S. patent application Ser. No. 09/803,713.

Another form of DNA strand displacement primer, referred to herein as a tertiary DNA strand displacement primer, is an oligonucleotide having sequence complementary to part of the sequence of an OCP or ATC. This sequence is referred to as the complementary portion of the tertiary DNA strand displacement primer. This complementary portion of the tertiary DNA strand displacement primer matches sequences in TS-DNA. The complementary portion of a tertiary DNA strand displacement primer may be complementary to any sequence in the OCP or ATC. However, it is preferred that it not be complementary OCP or ATC sequence matching the secondary DNA strand displacement primer. This prevents hybridization of the primers to each other. Preferably, the complementary portion of the tertiary DNA strand displacement primer has sequence complementary to a portion of the spacer portion of an OCP. The complementary portion of a tertiary DNA strand displacement primer can be any length that supports specific and stable hybridization between the primer and its complement. Generally this is 12 to 35 nucleotides long, but is preferably 18 to 25 nucleotides long. Tertiary DNA strand displacement primers can be fluorescent change primers although this is not preferred.

Useful tertiary DNA strand displacement primers for use in the disclosed method can form an intramolecular stem structure involving one or both of the tertiary DNA strand displacement primer's ends. Such tertiary DNA strand displacement primers are referred to herein as hairpin tertiary DNA strand displacement primers.

It is preferred that tertiary DNA strand displacement primers also contain additional sequence at their 5′ end that is not complementary to any part of the OCP or ATC. This sequence is referred to as the non-complementary portion of the tertiary DNA strand displacement primer. The non-complementary portion of the tertiary DNA strand displacement primer, if present, serves to facilitate strand displacement during DNA replication. The non-complementary portion of a tertiary DNA strand displacement primer may be any length, but is generally 1 to 100 nucleotides long, and preferably 4 to 8 nucleotides long. A rolling circle replication primer is a preferred form of tertiary DNA strand displacement primer. Tertiary DNA strand displacement primers can also comprise fluorescent moieties or labels and quenching moieties.

DNA strand displacement primers may also include modified nucleotides to make them resistant to exonuclease digestion. For example, the primer can have three or four phosphorothioate linkages between nucleotides at the 5′ end of the primer. Such nuclease resistant primers allow selective degradation of excess unligated OCP and gap oligonucleotides that might otherwise interfere with hybridization of detection probes, address probes, and secondary OCPs to the amplified nucleic acid. DNA strand displacement primers can be used for secondary DNA strand displacement and strand displacement cascade amplification, both described below and in U.S. Pat. No. 6,143,495.

D. Fluorescent Change Probes and Primers

Fluorescent change probes and fluorescent change primers refer to all probes and primers that involve a change in fluorescence intensity or wavelength based on a change in the form or conformation of the probe or primer and nucleic acid to be detected, assayed or replicated. Examples of fluorescent change probes and primers include molecular beacons, Amplifluors, FRET probes, cleavable FRET probes, TaqMan probes, scorpion primers, fluorescent triplex oligos including but not limited to triplex molecular beacons or triplex FRET probes, fluorescent water-soluble conjugated polymers, PNA probes and QPNA probes.

Fluorescent change probes and primers can be classified according to their structure and/or function. Fluorescent change probes include hairpin quenched probes, cleavage quenched probes, cleavage activated probes, and fluorescent activated probes. Fluorescent change primers include stem quenched primers and hairpin quenched primers. The use of several types of fluorescent change probes and primers are reviewed in Schweitzer and Kingsmore, Curr. Opin. Biotech. 12:21-27 (2001). Hall et al., Proc. Natl. Acad. Sci. USA 97:8272-8277 (2000), describe the use of fluorescent change probes with Invader assays.

Hairpin quenched probes are probes that when not bound to a target sequence form a hairpin structure (and, typically, a loop) that brings a fluorescent label and a quenching moiety into proximity such that fluorescence from the label is quenched. When the probe binds to a target sequence, the stem is disrupted, the quenching moiety is no longer in proximity to the fluorescent label and fluorescence increases. Examples of hairpin quenched probes are molecular beacons, fluorescent triplex oligos, triplex molecular beacons, triplex FRET probes, and QPNA probes.

Cleavage activated probes are probes where fluorescence is increased by cleavage of the probe. Cleavage activated probes can include a fluorescent label and a quenching moiety in proximity such that fluorescence from the label is quenched. When the probe is clipped or digested (typically by the 5′-3′ exonuclease activity of a polymerase during amplification), the quenching moiety is no longer in proximity to the fluorescent label and fluorescence increases. TaqMan probes (Holland et al., Proc. Natl. Acad. Sci. USA 88:7276-7280 (1991)) are an example of cleavage activated probes.

Cleavage quenched probes are probes where fluorescence is decreased or altered by cleavage of the probe. Cleavage quenched probes can include an acceptor fluorescent label and a donor moiety such that, when the acceptor and donor are in proximity, fluorescence resonance energy transfer from the donor to the acceptor causes the acceptor to fluoresce. The probes are thus fluorescent, for example, when hybridized to a target sequence. When the probe is clipped or digested (typically by the 5′-3′ exonuclease activity of a polymerase during amplification), the donor moiety is no longer in proximity to the acceptor fluorescent label and fluorescence from the acceptor decreases. If the donor moiety is itself a fluorescent label, it can release energy as fluorescence (typically at a different wavelength than the fluorescence of the acceptor) when not in proximity to an acceptor. The overall effect would then be a reduction of acceptor fluorescence and an increase in donor fluorescence. Donor fluorescence in the case of cleavage quenched probes is equivalent to fluorescence generated by cleavage activated probes with the acceptor being the quenching moiety and the donor being the fluorescent label. Cleavable FRET (fluorescence resonance energy transfer) probes are an example of cleavage quenched probes.

Fluorescent activated probes are probes or pairs of probes where fluorescence is increased or altered by hybridization of the probe to a target sequence. Fluorescent activated probes can include an acceptor fluorescent label and a donor moiety such that, when the acceptor and donor are in proximity (when the probes are hybridized to a target sequence), fluorescence resonance energy transfer from the donor to the acceptor causes the acceptor to fluoresce. Fluorescent activated probes are typically pairs of probes designed to hybridize to adjacent sequences such that the acceptor and donor are brought into proximity. Fluorescent activated probes can also be single probes containing both a donor and acceptor where, when the probe is not hybridized to a target sequence, the donor and acceptor are not in proximity but where the donor and acceptor are brought into proximity when the probe hybridized to a target sequence. This can be accomplished, for example, by placing the donor and acceptor on opposite ends a the probe and placing target complement sequences at each end of the probe where the target complement sequences are complementary to adjacent sequences in a target sequence. If the donor moiety of a fluorescent activated probe is itself a fluorescent label, it can release energy as fluorescence (typically at a different wavelength than the fluorescence of the acceptor) when not in proximity to an acceptor (that is, when the probes are not hybridized to the target sequence). When the probes hybridize to a target sequence, the overall effect would then be a reduction of donor fluorescence and an increase in acceptor fluorescence. FRET probes are an example of fluorescent activated probes.

Stem quenched primers are primers that when not hybridized to a complementary sequence form a stem structure (either an intramolecular stem structure or an intermolecular stem structure) that brings a fluorescent label and a quenching moiety into proximity such that fluorescence from the label is quenched. When the primer binds to a complementary sequence, the stem is disrupted, the quenching moiety is no longer in proximity to the fluorescent label and fluorescence increases. In the disclosed method, stem quenched primers are used as primers for nucleic acid synthesis and thus become incorporated into the synthesized or amplified nucleic acid. Examples of stem quenched primers are peptide nucleic acid quenched primers and hairpin quenched primers.

Peptide nucleic acid quenched primers are primers associated with a peptide nucleic acid quencher or a peptide nucleic acid fluor to form a stem structure. The primer contains a fluorescent label or a quenching moiety and is associated with either a peptide nucleic acid quencher or a peptide nucleic acid fluor, respectively. This puts the fluorescent label in proximity to the quenching moiety. When the primer is replicated, the peptide nucleic acid is displaced, thus allowing the fluorescent label to produce a fluorescent signal.

Hairpin quenched primers are primers that when not hybridized to a complementary sequence form a hairpin structure (and, typically, a loop) that brings a fluorescent label and a quenching moiety into proximity such that fluorescence from the label is quenched. When the primer binds to a complementary sequence, the stem is disrupted, the quenching moiety is no longer in proximity to the fluorescent label and fluorescence increases. Hairpin quenched primers are typically used as primers for nucleic acid synthesis and thus become incorporated into the synthesized or amplified nucleic acid. Examples of hairpin quenched primers are Amplifluor primers (Nazerenko et al., Nucleic Acids Res. 25:2516-2521 (1997)) and scorpion primers (Thelwell et al., Nucleic Acids Res. 28(19):3752-3761 (2000)).

Cleavage activated primers are similar to cleavage activated probes except that they are primers that are incorporated into replicated strands and are then subsequently cleaved. Little et al., Clin. Chem. 45:777-784 (1999), describe the use of cleavage activated primers.

E. Open Circle Probes

An open circle probe (OCP) is a linear DNA molecule. OCPs can be any length, but preferably contain between 50 to 1000 nucleotides, more preferably between about 60 to 150 nucleotides, and most preferably between about 70 to 100 nucleotides. The OCP has a 5′ phosphate group and a 3′ hydroxyl group. This allows the ends to be ligated (to each other or to other nucleic acid ends) using a ligase, coupled, or extended in a gap-filling operation. Open circle probes can be partially double-stranded. Useful open circle probes can comprise one or more primer complement portions, one or more secondary DNA strand displacement primer matching portions, and one or more detection tag portions.

Portions of the OCP can have specific functions making the OCP useful for RCA and LM-RCA. These portions are referred to as the target probe portions, the primer complement portions, the spacer region, the secondary DNA strand displacement primer matching portions, the detection tag portions, the secondary target sequence portions, the address tag portions, and the promoter portions. These portions are analogous to similarly-named portions of ATCs and their further description elsewhere herein in the context of ATCs is applicable to the analogous portion in OCPs. The target probe portions and at least one primer complement portion are required elements of an open circle probe. The primer complement portion can be part of, for example, the spacer region. Detection tag portions, secondary target sequence portions, promoter portions, and additional primer complement portions are optional and, when present, can be part of, for example, the spacer region. Address tag portions are optional and, when present, can be part of, for example, the spacer region. The primer complement portions, and the detection tag portions, the secondary target sequence portions, the address tag portions, and the promoter portions, if present, can be non-overlapping. However, various of these portions can be partially or completely overlapping if desired. OCPs can be single-stranded but may be partially double-stranded. In use, the target probe portions of an OCP should be single-stranded so that they can interact with target sequences.

Generally, an open circle probe can be a single-stranded, linear DNA molecule comprising, from 5′ end to 3′ end, a 5′ phosphate group, a right target probe portion, a spacer region, a left target probe portion, and a 3′ hydroxyl group, with a primer complement portion present as part of the spacer region. Particularly useful open circle probes can comprise a right target probe portion, a left target probe portion, one or more primer complement portions, and a secondary DNA strand displacement primer matching portion. Those segments of the spacer region that do not correspond to a specific portion of the OCP can be arbitrarily chosen sequences. For multiply-primed RCA, a plurality of primer complement portions are required. Where random or degenerate rolling circle replication primers are used, the sequence of the primer complement portions need not either be known or of a specified sequence. The open circle probe can include at least one detection tag portion when fluorescent change probes (or other detection probes) are used for detection.

It is preferred that OCPs do not have any sequences that are self-complementary. It is considered that this condition is met if there are no complementary regions greater than six nucleotides long without a mismatch or gap. It is also preferred that OCPs containing a promoter portion do not have any sequences that resemble a transcription terminator, such as a run of eight or more thymidine nucleotides. A lack of self-complementary sequences and a lack of promoter sequences is generally not required in the case of open circle probes including, derived from, or comprising nucleic acid molecules of interest. Such features will generally not be controlled for such open circle probes.

The open circle probe, when ligated and replicated, gives rise to a long DNA molecule containing multiple repeats of sequences complementary to the open circle probe. This long DNA molecule is referred to herein as tandem sequences DNA (TS-DNA). TS-DNA contains sequences complementary to the target probe portions, the primer complement portion, the spacer region, and, if present on the open circle probe, the detection tag portions, the secondary target sequence portions, the address tag portions, and the promoter portion. These sequences in the TS-DNA are referred to as target sequences (which match the original target sequence), primer sequences (which match the sequence of the rolling circle replication primer), spacer sequences (complementary to the spacer region), detection tags, secondary target sequences, address tags, and promoter sequences. The TS-DNA will also have sequence complementary to the matching portion of secondary DNA strand displacement primers. This sequence in the TS-DNA is referred to as the secondary DNA strand displacement primer complement or as the primer complement.

Preferably, the promoter portion of an OCP is immediately adjacent to the left target probe and is oriented to promote transcription toward the 3′ end of the open circle probe. This orientation results in transcripts that are complementary to TS-DNA, allowing independent detection of TS-DNA and the transcripts, and prevents transcription from interfering with rolling circle replication. Open circle probes can be capable of forming an intramolecular stem structure involving one or both of the OCP's ends. Such open circle probes are referred to herein as hairpin open circle probes. Open circle probes forming intramolecular stem structures, and their use in rolling circle amplification, are described in U.S. patent application Ser. No. 09/803,713.

1. Target Probe Portions

There are two target probe portions on each OCP, one at each end of the OCP. The target probe portions can each be any length that supports specific and stable hybridization between the target probes and the target sequence. For this purpose, a length of 10 to 35 nucleotides for each target probe portion is preferred, with target probe portions 15 to 25 nucleotides long being most preferred. The target probe portion at the 3′ end of the OCP is referred to as the left target probe, and the target probe portion at the 5′ end of the OCP is referred to as the right target probe. These target probe portions are also referred to herein as left and right target probes or left and right probes. The target probe portions are complementary to a target nucleic acid sequence.

The target probe portions are complementary to the target sequence, such that upon hybridization the 5′ end of the right target probe portion and the 3′ end of the left target probe portion are base-paired to adjacent nucleotides in the target sequence, with the objective that they serve as a substrate for ligation.

In another form of open circle probe, the 5′ end and the 3′ end of the target probe portions may hybridize in such a way that they are separated by a gap space. In this case the 5′ end and the 3′ end of the OCP may only be ligated if one or more additional oligonucleotides, referred to as gap oligonucleotides, are used, or if the gap space is filled during the ligation operation. The gap oligonucleotides hybridize to the target sequence in the gap space to form a continuous probe/target hybrid. The gap space may be any length desired but is generally ten nucleotides or less. It is preferred that the gap space is between about three to ten nucleotides in length, with a gap space of four to eight nucleotides in length being most preferred. Alternatively, a gap space could be filled using a DNA polymerase during the ligation operation. When using such a gap-filling operation, a gap space of three to five nucleotides in length is most preferred. As another alternative, the gap space can be partially bridged by one or more gap oligonucleotides, with the remainder of the gap filled using DNA polymerase.

F. Detection Labels

To aid in detection and quantitation of nucleic acids amplified using the disclosed method, detection labels can be directly incorporated into amplified nucleic acids or can be coupled to detection molecules. As used herein, a detection label is any molecule that can be associated with amplified nucleic acid, directly or indirectly, and which results in a measurable, detectable signal, either directly or indirectly. Many such labels for incorporation into nucleic acids or coupling to nucleic acid probes are known to those of skill in the art. Examples of detection labels suitable for use in the disclosed method are radioactive isotopes, fluorescent molecules, phosphorescent molecules, enzymes, antibodies, and ligands. Fluorescent labels, especially in the context of fluorescent change probes and primers, are useful for real-time detection of amplification.

Examples of suitable fluorescent labels include fluorescein isothiocyanate (FITC), 5,6-carboxymethyl fluorescein, Texas red, nitrobenz-2-oxa-1,3-diazol-4-yl (NBD), coumarin, dansyl chloride, rhodamine, amino-methyl coumarin (AMCA), Eosin, Erythrosin, BODIPY®, Cascade Blue®, Oregon Green®, pyrene, lissamine, xanthenes, acridines, oxazines, phycoerythrin, macrocyclic chelates of lanthamide ions such as quantum dye™, fluorescent energy transfer dyes, such as thiazole orange-ethidium heterodimer, and the cyanine dyes Cy3, Cy3.5, Cy5, Cy5.5 and Cy7. Examples of other specific fluorescent labels include 3-Hydroxypyrene 5,8,10-Tri Sulfonic acid, 5-Hydroxy Tryptamine (5-HT), Acid Fuchsin, Alizarin Complexon, Alizarin Red, Allophycocyanin, Aminocoumarin, Anthroyl Stearate, Astrazon Brilliant Red 4G, Astrazon Orange R, Astrazon Red 6B, Astrazon Yellow 7 GLL, Atabrine, Auramine, Aurophosphine, Aurophosphine G, BAO 9 (Bisaminophenyloxadiazole), BCECF, Berberine Sulphate, Bisbenzamide, Blancophor FFG Solution, Blancophor SV, Bodipy F1, Brilliant Sulphoflavin FF, Calcien Blue, Calcium Green, Calcofluor RW Solution, Calcofluor White, Calcophor White ABT Solution, Calcophor White Standard Solution, Carbostyryl, Cascade Yellow, Catecholamine, Chinacrine, Coriphosphine O, Coumarin-Phalloidin, CY3.1 8, CY5.1 8, CY7, Dans (1-Dimethyl Amino Naphaline 5 Sulphonic Acid), Dansa (Diamino Naphtyl Sulphonic Acid), Dansyl NH—CH3, Diamino Phenyl Oxydiazole (DAO), Dimethylamino-5-Sulphonic acid, Dipyrrometheneboron Difluoride, Diphenyl Brilliant Flavine 7GFF, Dopamine, Erythrosin ITC, Euchrysin, FIF (Formaldehyde Induced Fluorescence), Flazo Orange, Fluo 3, Fluorescamine, Fura-2, Genacryl Brilliant Red B, Genacryl Brilliant Yellow 10GF, Genacryl Pink 3G, Genacryl Yellow 5GF, Gloxalic Acid, Granular Blue, Haematoporphyrin, Indo-1, Intrawhite Cf Liquid, Leucophor PAF, Leucophor SF, Leucophor WS, Lissamine Rhodamine B200 (RD200), Lucifer Yellow CH, Lucifer Yellow VS, Magdala Red, Marina Blue, Maxilon Brilliant Flavin 10 GFF, Maxilon Brilliant Flavin 8 GFF, MPS (Methyl Green Pyronine Stilbene), Mithramycin, NBD Amine, Nitrobenzoxadidole, Noradrenaline, Nuclear Fast Red, Nuclear Yellow, Nylosan Brilliant Flavin E8G, Oxadiazole, Pacific Blue, Pararosaniline (Feulgen), Phorwite AR Solution, Phorwite BKL, Phorwite Rev, Phorwite RPA, Phosphine 3R, Phthalocyanine, Phycoerythrin R, Polyazaindacene Pontochrome Blue Black, Porphyrin, Primuline, Procion Yellow, Pyronine, Pyronine B, Pyrozal Brilliant Flavin 7GF, Quinacrine Mustard, Rhodamine 123, Rhodamine 5 GLD, Rhodamine 6G, Rhodamine B, Rhodamine B 200, Rhodamine B Extra, Rhodamine BB, Rhodamine BG, Rhodamine WT, Serotonin, Sevron Brilliant Red 2B, Sevron Brilliant Red 4G, Sevron Brilliant Red B, Sevron Orange, Sevron Yellow L, SITS (Primuline), SITS (Stilbene Isothiosulphonic acid), Stilbene, Snarf 1, sulpho Rhodamine B Can C, Sulpho Rhodamine G Extra, Tetracycline, Thiazine Red R, Thioflavin S, Thioflavin TCN, Thioflavin 5, Thiolyte, Thiozol Orange, Tinopol CBS, True Blue, Ultralite, Uranine B, Uvitex SFC, Xylene Orange, and XRITC.

Preferred fluorescent labels are fluorescein (5-carboxyfluorescein-N-hydroxysuccinimide ester), rhodamine (5,6-tetramethyl rhodamine), and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. Other examples of fluorescein dyes include 6-carboxyfluorescein (6-FAM), 2′,4′,1,4,-tetrachlorofluorescein (TET), 2′,4′,5′,7′,1,4-hexachlorofluorescein (HEX), 2′,7′-dimethoxy-4′,5′-dichloro-6-carboxyrhodamine (JOE), 2′-chloro-5′-fluoro-7′,8′-fused phenyl-1,4-dichloro-6-carboxyfluorescein (NED), and 2′-chloro-7′-phenyl-1,4-dichloro-6-carboxyfluorescein (VIC). Fluorescent labels can be obtained from a variety of commercial sources, including Amersham Pharmacia Biotech, Piscataway, N.J.; Molecular Probes, Eugene, Oreg.; and Research Organics, Cleveland, Ohio.

Additional labels of interest include those that provide for signal only when the probe with which they are associated is specifically bound to a target molecule, where such labels include: “molecular beacons” as described in Tyagi & Kramer, Nature Biotechnology (1996) 14:303 and EP 0 070 685 B1. Other labels of interest include those described in U.S. Pat. No. 5,563,037; WO 97/17471 and WO 97/17076.

Labeled nucleotides are a preferred form of detection label since they can be directly incorporated into the amplification products during synthesis. Examples of detection labels that can be incorporated into amplified nucleic acids include nucleotide analogs such as BrdUrd (5-bromodeoxyunridine, Hoy and Schimke, Mutation Research 290:217-230 (1993)), aminoallyldeoxyunridine (Henegariu et al., Nature Biotechnology 18:345-348 (2000)), 5-methylcytosine (Sano et al., Biochim. Biophys. Acta 951:157-165 (1988)), bromouridine (Wansick et al., J Cell Biology 122:283-293 (1993)) and nucleotides modified with biotin (Langer et al., Proc. Natl. Acad. Sci. USA 78:6633 (1981)) or with suitable haptens such as digoxygenin (Kerkhof, Anal. Biochem. 205:359-364 (1992)). Suitable fluorescence-labeled nucleotides are Fluorescein-isothiocyanate-dUTP, Cyanine-3-dUTP and Cyanine-5-dUTP (Yu et al., Nucleic Acids Res., 22:3226-3232 (1994)). A preferred nucleotide analog detection label for DNA is BrdUrd (bromodeoxyuridine, BrdUrd, BrdU, BUdR, Sigma-Aldrich Co). Other preferred nucleotide analogs for incorporation of detection label into DNA are AA-dUTP (aminoallyl-deoxyuridine triphosphate, Sigma-Aldrich Co.), and 5-methyl-dCTP (Roche Molecular Biochemicals). A preferred nucleotide analog for incorporation of detection label into RNA is biotin-16-UTP (biotin-16-uridine-5′-triphosphate, Roche Molecular Biochemicals). Fluorescein, Cy3, and Cy5 can be linked to dUTP for direct labeling. Cy3.5 and Cy7 are available as avidin or anti-digoxygenin conjugates for secondary detection of biotin- or digoxygenin-labeled probes.

Detection labels that are incorporated into amplified nucleic acid, such as biotin, can be subsequently detected using sensitive methods well-known in the art. For example, biotin can be detected using streptavidin-alkaline phosphatase conjugate (Tropix, Inc.), which is bound to the biotin and subsequently detected by chemiluminescence of suitable substrates (for example, chemiluminescent substrate CSPD: disodium, 3-(4-methoxyspiro-[1,2,-dioxetane-3-2′-(5′-chloro)tricyclo [3.3.1.1^{3,7}]decane]-4-yl)phenyl phosphate; Tropix, Inc.). Labels can also be enzymes, such as alkaline phosphatase, soybean peroxidase, horseradish peroxidase and polymerases, that can be detected, for example, with chemical signal amplification or by using a substrate to the enzyme which produces light (for example, a chemiluminescent 1,2-dioxetane substrate) or fluorescent signal.

Molecules that combine two or more of these detection labels are also considered detection labels. Any of the known detection labels can be used with the disclosed probes, tags, and method to label and detect nucleic acid amplified using the disclosed method. Methods for detecting and measuring signals generated by detection labels are also known to those of skill in the art. For example, radioactive isotopes can be detected by scintillation counting or direct visualization; fluorescent molecules can be detected with fluorescent spectrophotometers; phosphorescent molecules can be detected with a spectrophotometer or directly visualized with a camera; enzymes can be detected by detection or visualization of the product of a reaction catalyzed by the enzyme; antibodies can be detected by detecting a secondary detection label coupled to the antibody. As used herein, detection molecules are molecules which interact with amplified nucleic acid and to which one or more detection labels are coupled.

G. Nucleic Acid Molecules

The disclosed method can involve use of nucleic acid molecules and nucleic acid sequences as nucleic acid molecules of interest and as a source for target sequences and nucleic acid sequences of interest. Nucleic acid molecules of interest can be, or can be used in, amplification target circles. As used herein, unless the context indicates otherwise, the term nucleic acid molecule refers to both actual molecules and to nucleic acid sequences that are part of a larger nucleic acid molecule.

Nucleic acid molecule and sequences can be from any nucleic acid sample of interest. The source, identity, and preparation of many such nucleic acid samples are known. It is useful if nucleic acid samples known or identified for use in amplification or detection methods are used for the method described herein. The nucleic acid sample can be, for example, a nucleic acid sample from one or more cells, tissue, or bodily fluids such as blood, urine, semen, lymphatic fluid, cerebrospinal fluid, or amniotic fluid, or other biological samples, such as tissue culture cells, buccal swabs, mouthwash, stool, tissues slices, biopsy aspiration, and archeological samples such as bone or mummified tissue. Types of useful nucleic acid samples include blood samples, urine samples, semen samples, lymphatic fluid samples, cerebrospinal fluid samples, amniotic fluid samples, biopsy samples, needle aspiration biopsy samples, cancer samples, tumor samples, tissue samples, cell samples, cell lysate samples, a crude cell lysate samples, forensic samples, archeological samples, infection samples, nosocomial infection samples, production samples, drug preparation samples, biological molecule production samples, protein preparation samples, lipid preparation samples, and/or carbohydrate preparation samples.

Nucleic acid molecules and nucleic acid sequences that have or are sequences complementary to target probe portions of an open circle probe are also referred to as target molecules and target sequences. Examples of target molecules, target sequences, or sources of target sequences are mRNA molecules and cDNA molecules, although any nucleic acid molecule or sequence can be used in the disclosed compositions and method. Target sequences, which can be the object of amplification, can be any nucleic acid. Target sequences can include multiple nucleic acid molecules, such as in the case of mRNA amplification, multiple sites in a nucleic acid molecule, or a single region of a nucleic acid molecule. For example, target sequences can be mRNA and cDNA.

H. Nucleic Acid Samples

Nucleic acid samples can be derived from any source that has, or is suspected of having, nucleic acids. A nucleic acid sample is the source of nucleic acid molecules and nucleic acid sequences. Nucleic acid sample can contain, for example, a target nucleic acid, for example a specific mRNA or pool of mRNA molecules. The nucleic acid sample can contain RNA or DNA or both. The nucleic acid sample in certain embodiments can also include chemically synthesized nucleic acids. The nucleic acid sample can include any nucleotide, nucleotide analog, nucleotide substitute or nucleotide conjugate.

The nucleic acid sample can be, for example, a nucleic acid sample from one or more cells, tissue, or bodily fluids such as blood, urine, semen, lymphatic fluid, cerebrospinal fluid, or amniotic fluid, or other biological samples, such as tissue culture cells, buccal swabs, mouthwash, stool, tissues slices, biopsy aspiration, and archeological samples such as bone or mummified tissue. Types of useful nucleic acid samples include blood samples, urine samples, semen samples, lymphatic fluid samples, cerebrospinal fluid samples, amniotic fluid samples, biopsy samples, needle aspiration biopsy samples, cancer samples, tumor samples, tissue samples, cell samples, cell lysate samples, crude cell lysate samples, forensic samples, archeological samples, infection samples, nosocomial infection samples, production samples, drug preparation samples, biological molecule production samples, protein preparation samples, lipid preparation samples, and/or carbohydrate preparation samples.

I. Detection Probes

Detection probes are labeled oligonucleotides or oligomers having sequence complementary to detection tags on TS-DNA or transcripts of TS-DNA. The complementary portion of a detection probe can be any length that supports specific and stable hybridization between the detection probe and the detection tag. For this purpose, a length of 10 to 35 nucleotides is preferred, with a complementary portion of a detection probe 16 to 20 nucleotides long being most preferred. Detection probes can contain any of the detection labels described above. Preferred labels are biotin and fluorescent molecules. Useful detection probes are fluorescent change probes. A particularly preferred detection probe is a molecular beacon (which is a form of fluorescent change probe). Molecular beacons are detection probes labeled with fluorescent moieties where the fluorescent moieties fluoresce only when the detection probe is hybridized (Tyagi and Kramer, Nature Biotechnology 14:303-308 (1996)). The use of such probes eliminates the need for removal of unhybridized probes prior to label detection because the unhybridized detection probes will not produce a signal. This is especially useful in multiplex assays.

One form of detection probe, referred to herein as a collapsing detection probe, contains two separate complementary portions. This allows each detection probe to hybridize to two detection tags in TS-DNA. In this way, the detection probe forms a bridge between different parts of the TS-DNA. The combined action of numerous collapsing detection probes hybridizing to TS-DNA will be to form a collapsed network of cross-linked TS-DNA. Collapsed TS-DNA occupies a much smaller volume than free, extended TS-DNA, and includes whatever detection label present on the detection probe. This result is a compact and discrete detectable signal for each TS-DNA. Collapsing TS-DNA is useful both for in situ hybridization applications and for multiplex detection because it allows detectable signals to be spatially separate even when closely packed. Collapsing TS-DNA is described in U.S. Pat. No. 6,143,495.

J. Gap Oligonucleotides

Gap oligonucleotides are oligonucleotides that are complementary to all or a part of that portion of a target sequence which covers a gap space between the ends of a hybridized open circle probe. Gap oligonucleotides have a phosphate group at their 5′ ends and a hydroxyl group at their 3′ ends. This facilitates ligation of gap oligonucleotides to open circle probes, or to other gap oligonucleotides. The gap space between the ends of a hybridized open circle probe can be filled with a single gap oligonucleotide, or it can be filled with multiple gap oligonucleotides. For example, two 3 nucleotide gap oligonucleotides can be used to fill a six nucleotide gap space, or a three nucleotide gap oligonucleotide and a four nucleotide gap oligonucleotide can be used to fill a seven nucleotide gap space. Gap oligonucleotides are particularly useful for distinguishing between closely related target sequences. For example, multiple gap oligonucleotides can be used to amplify different allelic variants of a target sequence. By placing the region of the target sequence in which the variation occurs in the gap space formed by an open circle probe, a single open circle probe can be used to amplify each of the individual variants by using an appropriate set of gap oligonucleotides.

K. Reporter Binding Agents

A reporter binding agent is a specific binding molecule coupled or tethered to a nucleic acid such as an oligonucleotide. The specific binding molecule is referred to as the affinity portion of the reporter binding agent and the nucleic acid is referred to as the oligonucleotide portion of the reporter binding agent. As used herein, a specific binding molecule is a molecule that interacts specifically with a particular molecule or moiety (that is, an analyte). The molecule or moiety that interacts specifically with a specific binding molecule is referred to herein as a target molecule. The target molecules can be any analyte. It is to be understood that the term target molecule refers to both separate molecules and to portions of molecules, such as an epitope of a protein, that interacts specifically with a specific binding molecule. Antibodies, either member of a receptor/ligand pair, and other molecules with specific binding affinities are examples of specific binding molecules, useful as the affinity portion of a reporter binding molecule. A reporter binding molecule with an affinity portion which is an antibody is referred to herein as a reporter antibody. The oligonucleotide portion can be a nucleic acid molecule or a combination of nucleic acid molecules. The oligonucleotide portion is preferably an oligonucleotide or an amplification target circle.

By tethering an amplification target circle or coupling a target sequence to a specific binding molecule, binding of a specific binding molecule to its specific target can be detected by amplifying the ATC or target sequence with rolling circle amplification. This amplification allows sensitive detection of a very small number of bound specific binding molecules. A reporter binding molecule that interacts specifically with a particular target molecule is said to be specific for that target molecule. For example, a reporter binding molecule with an affinity portion which is an antibody that binds to a particular antigen is said to be specific for that antigen. The antigen is the target molecule. Reporter binding agents are also referred to herein as reporter binding molecules. FIGS. 25, 26, 27, 28, and 29 of U.S. Pat. No. 6,143,495 illustrate examples of several preferred types of reporter binding molecules and their use. FIG. 29 of U.S. Pat. No. 6,143,495 illustrates a reporter binding molecule using an antibody as the affinity portion.

Preferred target molecules are proteins and peptides. Use of reporter binding agents that target proteins and peptides allows sensitive signal amplification using rolling circle amplification for the detection of proteins and peptides. The ability to multiplex rolling circle amplification detection allows multiplex detection of the proteins and peptides (or any other target molecule). Thus, the disclosed method can be used for multi-protein analysis such as proteomics analysis. Such multi-protein analysis can be accomplished, for example, by using reporter binding agents targeted to different proteins, with the oligonucleotide portion of each reporter binding agent coded to allow separate amplification and detection of each different reporter binding agent.

In one embodiment, the oligonucleotide portion of a reporter binding agent can include an amplification target circle which serves as a template for rolling circle replication. In a multiplex assay using multiple reporter binding agents, it is preferred that primer complement portions, detection tag portions and/or whatever portions of the ATC comprising the oligonucleotide portion of each reporter binding agent that match or are complementary to a fluorescent change probe or primer be substantially different to aid unique detection of each reporter binding agent. Where fluorescent change probes are used, it is desirable to use the same primer complement portion in all of the ATCs used in a multiplex assay. The ATC is tethered to the specific binding molecule by looping the ATC around a tether loop. This allows the ATC to rotate freely during rolling circle replication while remaining coupled to the affinity portion. The tether loop can be any material that can form a loop and be coupled to a specific binding molecule. Linear polymers are a preferred material for tether loops.

A preferred method of producing a reporter binding agent with a tethered ATC is to form the tether loop by ligating the ends of oligonucleotides coupled to a specific binding molecule around an ATC. Oligonucleotides can be coupled to specific binding molecules using known techniques. For example, Hendrickson et al. (1995), describes a suitable method for coupling oligonucleotides to antibodies. This method is generally useful for coupling oligonucleotides to any protein. To allow ligation, oligonucleotides comprising the two halves of the tether loop should be coupled to the specific binding molecule in opposite orientations such that the free end of one is the 5′ end and the free end of the other is the 3′ end. Ligation of the ends of the tether oligonucleotides can be mediated by hybridization of the ends of the tether oligonucleotides to adjacent sequences in the ATC to be tethered. In this way, the ends of the tether oligonucleotides are analogous to the target probe portions of an open circle probe, with the ATC containing the target sequence. Similar techniques can be used to form tether loops containing a target sequence.

Another useful method of producing a reporter binding agent with a tethered ATC is to ligate an open circle probe while hybridized to an oligonucleotide tether loop on a specific binding molecule. In this method, both ends of a single tether oligonucleotide are coupled to a specific binding molecule. This can be accomplished using known coupling techniques as described above. Ligation of an open circle probe hybridized to a tether loop is analogous to the ligation operation of LM-RCA. In this case, the target sequence is part of an oligonucleotide with both ends coupled to a specific binding molecule. This same ligation technique can be used to circularize open circle probes on target sequences that are part of reporter binding agents. This topologically locks the open circle probe to the reporter binding agent (and thus, to the target molecule to which the reporter binding agent binds).

The ends of tether loops can be coupled to any specific binding molecule with functional groups that can be derivatized with suitable activating groups. When the specific binding molecule is a protein, or a molecule with similar functional groups, coupling of tether ends can be accomplished using known methods of protein attachment. Many such methods are described in Protein immobilization: fundamentals and applications Richard F. Taylor, ed. (M. Dekker, New York, 1991).

In another embodiment, the oligonucleotide portion of a reporter binding agent includes a sequence, referred to as a target sequence, that serves as a target sequence for an OCP. The sequence of the target sequence can be arbitrarily chosen. In a multiplex assay using multiple reporter binding agents, it is preferred that the target sequence for each reporter binding agent be substantially different to limit the possibility of non-specific target detection. Alternatively, it may be desirable in some multiplex assays, to use target sequences with related sequences. By using different, unique gap oligonucleotides to fill different gap spaces, such assays can use one or a few OCPs to amplify and detect a larger number of target sequences. The oligonucleotide portion can be coupled to the affinity portion by any of several established coupling reactions. For example, Hendrickson et al., Nucleic Acids Res., 23(3):522-529 (1995) describes a suitable method for coupling oligonucleotides to antibodies.

A preferred form of target sequence in a reporter binding agent is an oligonucleotide having both ends coupled to the specific binding molecule so as to form a loop. In this way, when the OCP hybridizes to the target and is circularized, the OCP will remain topologically locked to the reporter binding agent during rolling circle replication of the circularized OCP. This improves the localization of the resulting amplified signal to the location where the reporter binding agent is bound (that is, at the location of the target molecule).

A special form of reporter binding molecule, referred to herein as a reporter binding probe, has an oligonucleotide or oligonucleotide derivative as the specific binding molecule. Reporter binding probes are designed for and used to detect specific nucleic acid sequences. Thus, the target molecule for reporter binding probes are nucleic acid sequences. The target molecule for a reporter binding probe can be a nucleotide sequence within a larger nucleic acid molecule. It is to be understood that the term reporter binding molecule encompasses reporter binding probes. The specific binding molecule of a reporter binding probe can be any length that supports specific and stable hybridization between the reporter binding probe and the target molecule. For this purpose, a length of 10 to 40 nucleotides is preferred, with a specific binding molecule of a reporter binding probe 16 to 25 nucleotides long being most preferred.

It is preferred that the specific binding molecule of a reporter binding probe is peptide nucleic acid. Peptide nucleic acid forms a stable hybrid with DNA. This allows a reporter binding probe with a peptide nucleic acid specific binding molecule to remain firmly adhered to the target sequence during subsequent amplification and detection operations. This useful effect can also be obtained with reporter binding probes with oligonucleotide specific binding molecules by making use of the triple helix chemical bonding technology described by Gasparro et al., Nucleic Acids Res. 1994 22(14):2845-2852 (1994). Briefly, the affinity portion of a reporter binding probe is designed to form a triple helix when hybridized to a target sequence. This is accomplished generally as known, preferably by selecting either a primarily homopurine or primarily homopyrimidine target sequence. The matching oligonucleotide sequence which constitutes the affinity portion of the reporter binding probe will be complementary to the selected target sequence and thus be primarily homopyrimidine or primarily homopurine, respectively. The reporter binding probe (corresponding to the triple helix probe described by Gasparro et al.) contains a chemically linked psoralen derivative. Upon hybridization of the reporter binding probe to a target sequence, a triple helix forms. By exposing the triple helix to low wavelength ultraviolet radiation, the psoralen derivative mediates cross-linking of the probe to the target sequence. FIGS. 25, 26, 27, and 28 of U.S. Pat. No. 6,143,495 illustrate examples of reporter binding molecules that are reporter binding probes.

The specific binding molecule in a reporter binding probe can also be a bipartite DNA molecule, such as ligatable DNA probes adapted from those described by Landegren et al., Science 241:1077-1080 (1988). When using such a probe, the affinity portion of the probe is assembled by target-mediated ligation of two oligonucleotide portions which hybridize to adjacent regions of a target nucleic acid. Thus, the components used to form the affinity portion of such reporter binding probes are a truncated reporter binding probe (with a truncated affinity portion which hybridizes to part of the target sequence) and a ligation probe which hybridizes to an adjacent part of the target sequence such that it can be ligated to the truncated reporter binding probe. The ligation probe can also be separated from (that is, not adjacent to) the truncated reporter binding probe when both are hybridized to the target sequence. The resulting space between them can then be filled by a second ligation probe or by gap-filling synthesis. For use in the disclosed methods, it is preferred that the truncated affinity portion be long enough to allow target-mediated ligation but short enough to, in the absence of ligation to the ligation probe, prevent stable hybridization of the truncated reporter binding probe to the target sequence during the subsequent amplification operation. For this purpose, a specific step designed to eliminate hybrids between the target sequence and unligated truncated reporter binding probes can be used following the ligation operation.

In another embodiment, the oligonucleotide portion of a reporter binding agent includes a sequence, referred to as a rolling circle replication primer sequence, that serves as a rolling circle replication primer for an ATC. This allows rolling circle replication of an added ATC where the resulting TS-DNA is coupled to the reporter binding agent. Because of this, the TS-DNA will be effectively immobilized at the site of the target molecule. Preferably, the immobilized TS-DNA can then be collapsed in situ prior to detection. The sequence of the rolling circle replication primer sequence can be arbitrarily chosen. The rolling circle replication sequence can be designed to form and intramolecular stem structure as described for rolling circle replication primers above. Additional, untethered rolling circle replication primers can also be used to achieve multiply-primed RCA.

In a multiplex assay using multiple reporter binding agents, it is preferred that the fluorescent change probes or primers used with each reporter binding agent be substantially different to limit the possibility of non-specific target detection. Alternatively, it may be desirable in some multiplex assays, to use fluorescent change probes or primers with related sequences. Such assays can use one or a few ATCs to detect a larger number of target molecules. Any of the other relationships between ATCs and primers and probes disclosed herein can also be used. When the oligonucleotide portion of a reporter binding agent is used as a rolling circle replication primer, the oligonucleotide portion can be any length that supports specific and stable hybridization between the oligonucleotide portion and the primer complement portion of an amplification target circle. Generally this is 10 to 35 nucleotides long, but is preferably 16 to 20 nucleotides long. FIGS. 25, 26, 27, 28, and 29 of U.S. Pat. No. 6,143,495 illustrate examples of reporter binding molecules in which the oligonucleotide portion is a rolling circle replication primer.

Antibodies useful as the affinity portion of reporter binding agents, can be obtained commercially or produced using well established methods. For example, Johnstone and Thorpe, on pages 30-85, describe general methods useful for producing both polyclonal and monoclonal antibodies. The entire book describes many general techniques and principles for the use of antibodies in assay systems.

L. Address Probes

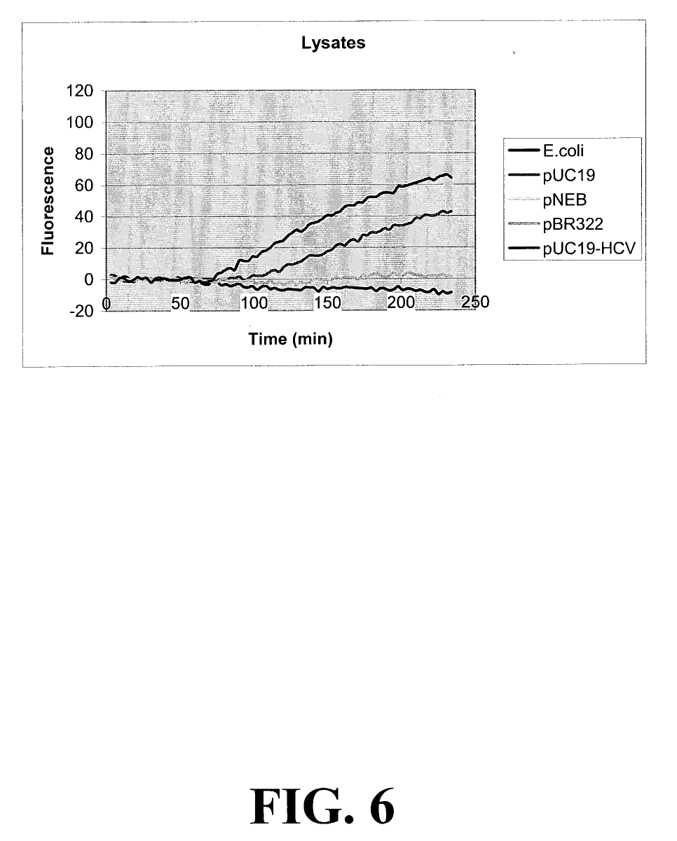

An address probe is an oligonucleotide or oligomer having a sequence complementary to address tags on TS-DNA or transcripts of TS-DNA. The complementary portion of an address probe can be any length that supports specific and stable hybridization between the address probe and the address tag. For this purpose, a length of 10 to 35 nucleotides is preferred, with a complementary portion of an address probe 12 to 18 nucleotides long being most preferred. Preferably, the complementary portion of an address probe is complementary to all or a portion of the target probe portions of an OCP. Most preferably, the complementary portion of an address probe is complementary to a portion of either or both of the left and right target probe portions of an OCP and all or a part of any gap oligonucleotides or gap sequence created in a gap-filling operation (see FIG. 6 of U.S. Pat. No. 6,143,495). Address probe can contain a single complementary portion or multiple complementary portions. Preferably, address probes are coupled, either directly or via a spacer molecule, to a solid-state support. Such a combination of address probe and solid-state support are a preferred form of solid-state detector. Address probes can be fluorescent change probes although this is not preferred.

M. Solid Supports

Solid supports are solid-state substrates or supports with which amplification products of the disclosed method (or other components used in, or produced by, the disclosed method) can be associated. Amplification products can be associated with solid supports directly or indirectly. For example, amplification products can be bound to the surface of a solid support or associated with address probes, or detection probes immobilized on solid supports. An array detector is a solid support to which multiple different address probes or detection probes have been coupled in an array, grid, or other organized pattern. Target molecules and target sequences can also be attached to solid supports.

Solid-state substrates for use in solid supports can include any solid material with which components can be associated, directly or indirectly. This includes materials such as acrylamide, agarose, cellulose, nitrocellulose, glass, gold, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, functionalized silane, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or a combination. Solid-state substrates and solid supports can be porous or non-porous. A chip is a rectangular or square small piece of material. Preferred forms for solid-state substrates are thin films, beads, or chips. A useful form for a solid-state substrate is a microtiter dish. In some embodiments, a multiwell glass slide can be employed.

Different address probes and/or detection probes can be used together as a set. The set can be used as a mixture of all or subsets of the address probes and/or detection probes used separately in separate reactions, or immobilized on a solid support. Address probes and/or detection probes used separately or as mixtures can be physically separable through, for example, association with or immobilization on a solid support. An array can include a plurality of address probes and/or detection probes immobilized at identified or predefined locations on the solid support. Each predefined location on the solid support generally has one type of component (that is, all the components at that location are the same). Alternatively, multiple types of components can be immobilized in the same predefined location on a solid support. Each location will have multiple copies of the given components. The spatial separation of different components on the solid support allows separate detection and identification of amplification products.

Although useful, it is not required that the solid support be a single unit or structure. The set of analytes, analyte capture agents, or accessory molecules may be distributed over any number of solid supports. For example, at one extreme, each probe may be immobilized in a separate reaction tube or container, or on separate beads or microparticles.

Methods for immobilization of oligonucleotides to solid-state substrates are well established. Oligonucleotides, including address probes and detection probes, can be coupled to substrates using established coupling methods. For example, suitable attachment methods are described by Pease et al., Proc. Natl. Acad. Sci. USA 91(11):5022-5026 (1994), and Khrapko et al., Mol Biol (Mosk) (USSR) 25:718-730 (1991). A method for immobilization of 3′-amine oligonucleotides on casein-coated slides is described by Stimpson et al., Proc. Natl. Acad. Sci. USA 92:6379-6383 (1995). A useful method of attaching oligonucleotides to solid-state substrates is described by Guo et al., Nucleic Acids Res. 22:5456-5465 (1994).

Each of the components (for example, address probes and/or detection probes) immobilized on the solid support can be located in a different predefined region of the solid support. The different locations can be different reaction chambers. Each of the different predefined regions can be physically separated from each other of the different regions. The distance between the different predefined regions of the solid support can be either fixed or variable. For example, in an array, each of the components can be arranged at fixed distances from each other, while components associated with beads will not be in a fixed spatial relationship. In particular, the use of multiple solid support units (for example, multiple beads) will result in variable distances.

Components can be associated or immobilized on a solid support at any density. Components can be immobilized to the solid support at a density exceeding 400 different components per cubic centimeter. Arrays of components can have any number of components. For example, an array can have at least 1,000 different components immobilized on the solid support, at least 10,000 different components immobilized on the solid support, at least 100,000 different components immobilized on the solid support, or at least 1,000,000 different components immobilized on the solid support.

N. Solid-State Detectors

Solid-state detectors are solid supports to which address probes or detection molecules have been coupled. A preferred form of solid-state detector is an array detector. An array detector is a solid-state detector to which multiple different address probes or detection molecules have been coupled in an array, grid, or other organized pattern.

Solid-state substrates for use in solid-state detectors can include any solid material to which oligonucleotides can be coupled. This includes materials such as acrylamide, agarose, cellulose, nitrocellulose, glass, gold, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, functionalized silane, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or a combination. Solid-state substrates and solid supports can be porous or non-porous. A chip is a rectangular or square small piece of material. Preferred forms for solid-state substrates are thin films, beads, or chips. A useful form for a solid-state substrate is a microtiter dish. In some embodiments, a multiwell glass slide can be employed.

Address probes immobilized on a solid-state substrate allow capture of the products of the disclosed amplification method on a solid-state detector. Such capture provides a convenient means of washing away reaction components that might interfere with subsequent detection steps. By attaching different address probes to different regions of a solid-state detector, different amplification products can be captured at different, and therefore diagnostic, locations on the solid-state detector. For example, in a multiplex assay, address probes specific for numerous different amplified nucleic acids (each representing a different target sequence amplified via a different set of primers) can be immobilized in an array, each in a different location. Capture and detection will occur only at those array locations corresponding to amplified nucleic acids for which the corresponding target sequences were present in a sample.

Methods for immobilization of oligonucleotides to solid-state substrates are well established. Oligonucleotides, including address probes and detection probes, can be coupled to substrates using established coupling methods. For example, suitable attachment methods are described by Pease et al., Proc. Natl. Acad. Sci. USA 91(11):5022-5026 (1994), and Khrapko et al., Mol Biol (Mosk) (USSR) 25:718-730 (1991). A method for immobilization of 3′-amine oligonucleotides on casein-coated slides is described by Stimpson et al., Proc. Natl. Acad. Sci. USA 92:6379-6383 (1995). A preferred method of attaching oligonucleotides to solid-state substrates is described by Guo et al., Nucleic Acids Res. 22:5456-5465 (1994). Examples of nucleic acid chips and arrays, including methods of making and using such chips and arrays, are described in U.S. Pat. No. 6,287,768, U.S. Pat. No. 6,288,220, U.S. Pat. No. 6,287,776, U.S. Pat. No. 6,297,006, and U.S. Pat. No. 6,291,193.

Some solid-state detectors useful in the disclosed method have detection antibodies attached to a solid-state substrate. Such antibodies can be specific for a molecule of interest. Captured molecules of interest can then be detected by binding of a second, reporter antibody, followed by amplification. Such a use of antibodies in a solid-state detector allows amplification assays to be developed for the detection of any molecule for which antibodies can be generated. Methods for immobilizing antibodies to solid-state substrates are well established. Immobilization can be accomplished by attachment, for example, to aminated surfaces, carboxylated surfaces or hydroxylated surfaces using standard immobilization chemistries. Examples of attachment agents are cyanogen bromide, succinimide, aldehydes, tosyl chloride, avidin-biotin, photocrosslinkable agents, epoxides and maleimides. A preferred attachment agent is glutaraldehyde. These and other attachment agents, as well as methods for their use in attachment, are described in Protein immobilization: fundamentals and applications, Richard F. Taylor, ed. (M. Dekker, New York, 1991), Johnstone and Thorpe, Immunochemistry In Practice (Blackwell Scientific Publications, Oxford, England, 1987) pages 209-216 and 241-242, and Immobilized Affinity Ligands, Craig T. Hermanson et al., eds. (Academic Press, New York, 1992). Antibodies can be attached to a substrate by chemically cross-linking a free amino group on the antibody to reactive side groups present within the solid-state substrate. For example, antibodies may be chemically cross-linked to a substrate that contains free amino or carboxyl groups using glutaraldehyde or carbodiimides as cross-linker agents. In this method, aqueous solutions containing free antibodies are incubated with the solid-state substrate in the presence of glutaraldehyde or carbodiimide. For crosslinking with glutaraldehyde the reactants can be incubated with 2% glutaraldehyde by volume in a buffered solution such as 0.1 M sodium cacodylate at pH 7.4. Other standard immobilization chemistries are known by those of skill in the art.

O. Solid-State Samples

Solid-state samples are solid supports to which target molecules or target sequences have been coupled or adhered. Target molecules or target sequences are preferably delivered in a target sample or assay sample. A preferred form of solid-state sample is an array sample. An array sample is a solid-state sample to which multiple different target samples or assay samples have been coupled or adhered in an array, grid, or other organized pattern.

Solid-state substrates for use in solid-state samples can include any solid material to which target molecules or target sequences can be coupled or adhered. This includes materials such as acrylamide, agarose, cellulose, nitrocellulose, glass, gold, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, functionalized silane, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or a combination. Solid-state substrates and solid supports can be porous or non-porous. A chip is a rectangular or square small piece of material. Preferred forms for solid-state substrates are thin films, beads, or chips. A useful form for a solid-state substrate is a microtiter dish. In some embodiments, a multiwell glass slide can be employed.

Target molecules and target sequences immobilized on a solid-state substrate allow formation of target-specific TS-DNA localized on the solid-state substrate. Such localization provides a convenient means of washing away reaction components that might interfere with subsequent detection steps, and a convenient way of assaying multiple different samples simultaneously. Diagnostic TS-DNA can be independently formed at each site where a different sample is adhered. For immobilization of target sequences or other oligonucleotide molecules to form a solid-state sample, the methods described above for can be used. Nucleic acids produced in the disclosed method can be coupled or adhered to a solid-state substrate in any suitable way. For example, nucleic acids generated by multiple strand displacement can be attached by adding modified nucleotides to the 3′ ends of nucleic acids produced by strand displacement replication using terminal deoxynucleotidyl transferase, and reacting the modified nucleotides with a solid-state substrate or support thereby attaching the nucleic acids to the solid-state substrate or support.

A preferred form of solid-state substrate is a glass slide to which up to 256 separate target samples have been adhered as an array of small dots. Each dot is preferably from 0.1 to 2.5 mm in diameter, and most preferably around 2.5 mm in diameter. Such microarrays can be fabricated, for example, using the method described by Schena et al., Science 270:487-470 (1995). Briefly, microarrays can be fabricated on poly-L-lysine-coated microscope slides (Sigma) with an arraying machine fitted with one printing tip. The tip is loaded with 1 μl of a DNA sample (0.5 mg/ml) from, for example, 96-well microtiter plates and deposited ˜0.005 μl per slide on multiple slides at the desired spacing. The printed slides can then be rehydrated for 2 hours in a humid chamber, snap-dried at 100° C. for 1 minute, rinsed in 0.1% SDS, and treated with 0.05% succinic anhydride prepared in buffer consisting of 50% 1-methyl-2-pyrrolidinone and 50% boric acid. The DNA on the slides can then be denatured in, for example, distilled water for 2 minutes at 90° C. immediately before use. Microarray solid-state samples can scanned with, for example, a laser fluorescent scanner with a computer-controlled XY stage and a microscope objective. A mixed gas, multiline laser allows sequential excitation of multiple fluorophores.

P. DNA polymerases

DNA polymerases useful in the rolling circle replication step of the disclosed method must perform rolling circle replication of primed circular templates. Such polymerases are referred to herein as rolling circle DNA polymerases. For rolling circle replication, it is preferred that a DNA polymerase be capable of displacing the strand complementary to the template strand, termed strand displacement, and lack a 5′ to 3′ exonuclease activity. Strand displacement is necessary to result in synthesis of multiple tandem copies of the ligated OCP. A 5′ to 3′ exonuclease activity, if present, might result in the destruction of the synthesized strand. DNA polymerases for use in the disclosed method can also be highly processive, if desired. The suitability of a DNA polymerase for use in the disclosed method can be readily determined by assessing its ability to carry out rolling circle replication. Preferred rolling circle DNA polymerases are Bst DNA polymerase, VENT® DNA polymerase (Kong et al., J. Biol. Chem. 268:1965-1975 (1993)), ThermoSequenase™, delta Tts DNA polymerase, Bca DNA polymerase (Journal of Biochemistry 113(3):401-10, 1993 Mar.), bacteriophage φ29 DNA polymerase (U.S. Pat. Nos. 5,198,543 and 5,001,050 to Blanco et al.), phage M2 DNA polymerase (Matsumoto et al., Gene 84:247 (1989)), phage φPRD1 DNA polymerase (Jung et al., Proc. Natl. Acad. Sci. USA 84:8287 (1987)), Klenow fragment of DNA polymerase I (Jacobsen et al., Eur. J. Biochem. 45:623-627 (1974)), T5 DNA polymerase (Chatterjee et al., Gene 97:13-19 (1991)), PRD1 DNA polymerase (Zhu and Ito, Biochim. Biophys. Acta. 1219:267-276 (1994)), modified T7 DNA polymerase (Tabor and Richardson, J. Biol. Chem. 262:15330-15333 (1987); Tabor and Richardson, J. Biol. Chem. 264:6447-6458 (1989); Sequenase™ (U.S. Biochemicals)), and T4 DNA polymerase holoenzyme (Kaboord and Benkovic, Curr. Biol. 5:149-157 (1995)). More preferred are Bst DNA polymerase, VENT® DNA polymerase, ThermoSequenase™, and delta Tts DNA polymerase. Bst DNA polymerase is most preferred.

Strand displacement can be facilitated through the use of a strand displacement factor, such as helicase. It is considered that any DNA polymerase that can perform rolling circle replication in the presence of a strand displacement factor is suitable for use in the disclosed method, even if the DNA polymerase does not perform rolling circle replication in the absence of such a factor. Strand displacement factors useful in the disclosed method include BMRF1 polymerase accessory subunit (Tsurumi et al., J Virology 67(12):7648-7653 (1993)), adenovirus DNA-binding protein (Zijderveld and van der Vliet, J Virology 68(2):1158-1164 (1994)), herpes simplex viral protein ICP8 (Boehmer and Lehman, J Virology 67(2):711-715 (1993); Skaliter and Lehman, Proc. Natl. Acad. Sci. USA 91(22):10665-10669 (1994)), single-stranded DNA binding proteins (SSB; Rigler and Romano, J. Biol. Chem. 270:8910-8919 (1995)), and calf thymus helicase (Siegel et al., J. Biol. Chem. 267:13629-13635 (1992)).

The ability of a polymerase to carry out rolling circle replication can be determined by using the polymerase in a rolling circle replication assay such as those described in Fire and Xu, Proc. Natl. Acad. Sci. USA 92:4641-4645 (1995) and in U.S. Pat. No. 6,143,495 (Example 1).

Another type of DNA polymerase can be used if a gap-filling synthesis step is used, such as in gap-filling LM-RCA (see U.S. Pat. No. 6,143,495, Example 3). When using a DNA polymerase to fill gaps, strand displacement by the DNA polymerase is undesirable. Such DNA polymerases are referred to herein as gap-filling DNA polymerases. Unless otherwise indicated, a DNA polymerase referred to herein without specifying it as a rolling circle DNA polymerase or a gap-filling DNA polymerase, is understood to be a rolling circle DNA polymerase and not a gap-filling DNA polymerase. Preferred gap-filling DNA polymerases are T7 DNA polymerase (Studier et al., Methods Enzymol. 185:60-89 (1990)), DEEP VENT® DNA polymerase (New England Biolabs, Beverly, Mass.), modified T7 DNA polymerase (Tabor and Richardson, J. Biol. Chem. 262:15330-15333 (1987); Tabor and Richardson, J. Biol. Chem. 264:6447-6458 (1989); Sequenase™ (U.S. Biochemicals)), and T4 DNA polymerase (Kunkel et al., Methods Enzymol. 154:367-382 (1987)). An especially preferred type of gap-filling DNA polymerase is the Thermus flavus DNA polymerase (MBR, Milwaukee, Wis.). The most preferred gap-filling DNA polymerase is the Stoffel fragment of Taq DNA polymerase (Lawyer et al., PCR Methods Appl. 2(4):275-287 (1993), King et al., J. Biol. Chem. 269(18):13061-13064 (1994)).

The ability of a polymerase to fill gaps can be determined by performing gap-filling LM-RCA. Gap-filling LM-RCA is performed with an open circle probe that forms a gap space when hybridized to the target sequence. Ligation can only occur when the gap space is filled by the DNA polymerase. If gap-filling occurs, TS-DNA can be detected, otherwise it can be concluded that the DNA polymerase, or the reaction conditions, is not useful as a gap-filling DNA polymerase.

Q. DNA ligases

Any DNA ligase is suitable for use in the disclosed amplification method. Preferred ligases are those that preferentially form phosphodiester bonds at nicks in double-stranded DNA. That is, ligases that fail to ligate the free ends of single-stranded DNA at a significant rate are preferred. Thermostable ligases are especially preferred. Many suitable ligases are known, such as T4 DNA ligase (Davis et al., Advanced Bacterial Genetics—A Manual for Genetic Engineering (Cold Spring Harbor Laboratory, Cold Spring Harbor, N.Y., 1980)), E. coli DNA ligase (Panasnko et al., J. Biol. Chem. 253:4590-4592 (1978)), AMPLIGASE® (Kalin et al., Mutat. Res., 283(2):119-123 (1992); Winn-Deen et al., Mol Cell Probes (England) 7(3):179-186 (1993)), Taq DNA ligase (Barany, Proc. Natl. Acad. Sci. USA 88:189-193 (1991), Thermus thermophilus DNA ligase (Abbott Laboratories), Thermus scotoductus DNA ligase and Rhodothermus marinus DNA ligase (Thorbjarnardottir et al., Gene 151:177-180 (1995)). T4 DNA ligase is preferred for ligations involving RNA target sequences due to its ability to ligate DNA ends involved in DNA:RNA hybrids (Hsuih et al., Quantitative detection of HCV RNA using novel ligation-dependent polymerase chain reaction, American Association for the Study of Liver Diseases (Chicago, Ill., Nov. 3-7, 1995)).

The frequency of non-target-directed ligation catalyzed by a ligase can be determined as follows. LM-RCA is performed with an open circle probe and a gap oligonucleotide in the presence of a target sequence. Non-targeted-directed ligation products can then be detected by using an address probe specific for the open circle probe ligated without the gap oligonucleotide to capture TS-DNA from such ligated probes. Target directed ligation products can be detected by using an address probe specific for the open circle probe ligated with the gap oligonucleotide. By using a solid-state detector with regions containing each of these address probes, both target directed and non-target-directed ligation products can be detected and quantitated. The ratio of target-directed and non-target-directed TS-DNA produced provides a measure of the specificity of the ligation operation. Target-directed ligation can also be assessed as discussed in Barany (1991).

R. RNA polymerases

Any RNA polymerase which can carry out transcription in vitro and for which promoter sequences have been identified can be used in the disclosed rolling circle transcription method. Stable RNA polymerases without complex requirements are preferred. Most preferred are T7 RNA polymerase (Davanloo et al., Proc. Natl. Acad. Sci. USA 81:2035-2039 (1984)) and SP6 RNA polymerase (Butler and Chamberlin, J. Biol. Chem. 257:5772-5778 (1982)) which are highly specific for particular promoter sequences (Schenbom and Meirendorf, Nucleic Acids Research 13:6223-6236 (1985)). Other RNA polymerases with this characteristic are also preferred. Because promoter sequences are generally recognized by specific RNA polymerases, the OCP or ATC should contain a promoter sequence recognized by the RNA polymerase that is used. Numerous promoter sequences are known and any suitable RNA polymerase having an identified promoter sequence can be used. Promoter sequences for RNA polymerases can be identified using established techniques.

S. Oligonucleotide Synthesis

Amplification target circles, rolling circle replication primers, detection probes, address probes, DNA strand displacement primers, open circle probes, gap oligonucleotides and any other oligonucleotides can be synthesized using established oligonucleotide synthesis methods. Methods to produce or synthesize oligonucleotides are well known. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method. Solid phase chemical synthesis of DNA fragments is routinely performed using protected nucleoside cyanoethyl phosphoramidites (S. L. Beaucage et al. (1981) Tetrahedron Lett. 22:1859). In this approach, the 3′-hydroxyl group of an initial 5′-protected nucleoside is first covalently attached to the polymer support (R. C. Pless et al. (1975) Nucleic Acids Res. 2:773 (1975)). Synthesis of the oligonucleotide then proceeds by deprotection of the 5′-hydroxyl group of the attached nucleoside, followed by coupling of an incoming nucleoside-3′-phosphoramidite to the deprotected hydroxyl group (M. D. Matteucci et a. (1981) J. Am. Chem. Soc. 103:3185). The resulting phosphite triester is finally oxidized to a phosphorotriester to complete the internucleotide bond (R. L. Letsinger et al. (1976) J. Am. Chem. Soc. 9:3655). Alternatively, the synthesis of phosphorothioate linkages can be carried out by sulfurization of the phosphite triester. Several chemicals can be used to perform this reaction, among them 3H-1,2-benzodithiole-3-one, 1,1-dioxide (R. P. Iyer, W. Egan, J. B. Regan, and S. L. Beaucage, J. Am. Chem. Soc., 1990, 112, 1253-1254). The steps of deprotection, coupling and oxidation are repeated until an oligonucleotide of the desired length and sequence is obtained. Other methods exist to generate oligonucleotides such as the H-phosphonate method (Hall et al, (1957) J. Chem. Soc., 3291-3296) or the phosphotriester method as described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994). Other forms of oligonucleotide synthesis are described in U.S. Pat. No. 6,294,664 and U.S. Pat. No. 6,291,669.

The nucleotide sequence of an oligonucleotide is generally determined by the sequential order in which subunits of subunit blocks are added to the oligonucleotide chain during synthesis. Each round of addition can involve a different, specific nucleotide precursor, or a mixture of one or more different nucleotide precursors. In general, degenerate or random positions in an oligonucleotide can be produced by using a mixture of nucleotide precursors representing the range of nucleotides that can be present at that position. Thus, precursors for A and T can be included in the reaction for a particular position in an oligonucleotide if that position is to be degenerate for A and T. Precursors for all four nucleotides can be included for a fully degenerate or random position. Completely random oligonucleotides can be made by including all four nucleotide precursors in every round of synthesis. Degenerate oligonucleotides can also be made having different proportions of different nucleotides. Such oligonucleotides can be made, for example, by using different nucleotide precursors, in the desired proportions, in the reaction.

Many of the oligonucleotides described herein are designed to be complementary to certain portions of other oligonucleotides or nucleic acids such that stable hybrids can be formed between them. The stability of these hybrids can be calculated using known methods such as those described in Lesnick and Freier, Biochemistry 34:10807-10815 (1995), McGraw et al., Biotechniques 8:674-678 (1990), and Rychlik et al., Nucleic Acids Res. 18:6409-6412 (1990).

Oligonucleotides can be synthesized, for example, on a Perseptive Biosystems 8909 Expedite Nucleic Acid Synthesis system using standard β-cyanoethyl phosphoramidite coupling chemistry on synthesis columns (Glen Research, Sterling, Va.). Oxidation of the newly formed phosphites can be carried out using, for example, the sulfurizing reagent 3H-1,2-benzothiole-3-one-1,1-idoxide (Glen Research) or the standard oxidizing reagent after the first and second phosphoramidite addition steps. The thio-phosphitylated oligonucleotides can be deprotected, for example, using 30% ammonium hydroxide (3.0 ml) in water at 55° C. for 16 hours, concentrated in an OP 120 Savant Oligo Prep deprotection unit for 2 hours, and desalted with PD10 Sephadex columns using the protocol provided by the manufacturer.

Hexamer oligonucleotides can be synthesized on a Perseptive Biosystems 8909 Expedite Nucleic Acid Synthesis system using standard β-cyanoethyl phosphoramidite coupling chemistry on mixed dA+dC+dG+dT synthesis columns (Glen Research, Sterling, Va.). The four phosphoramidites can be mixed in equal proportions to randomize the bases at each position in the oligonucleotide. Oxidation of the newly formed phosphites can be carried out using the sulfurizing reagent 3H-1,2-benzothiole-3-one-1,1-idoxide (Glen Research) instead of the standard oxidizing reagent after the first and second phosphoramidite addition steps. The thio-phosphitylated oligonucleotides can be deprotected using 30% ammonium hydroxide (3.0 ml) in water at 55° C. for 16 hours, concentrated in an OP 120 Savant Oligo Prep deprotection unit for 2 hours, and desalted with PD 10 Sephadex columns using the protocol provided by the manufacturer.

So long as their relevant function is maintained, amplification target circles, rolling circle replication primers, detection probes, address probes, DNA strand displacement primers, open circle probes, gap oligonucleotides and any other oligonucleotides can be made up of or include modified nucleotides (nucleotide analogs). Many modified nucleotides are known and can be used in oligonucleotides. A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl, hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine. 5-methylcytosine can increase the stability of duplex formation. Other modified bases are those that function as universal bases. Universal bases include 3-nitropyrrole and 5-nitroindole. Universal bases substitute for the normal bases but have no bias in base pairing. That is, universal bases can base pair with any other base. Base modifications often can be combined with for example a sugar modification, such as 2′-O-methoxyethyl, to achieve unique properties such as increased duplex stability. There are numerous United States patents such as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, which detail and describe a range of base modifications. Each of these patents is herein incorporated by reference in its entirety, and specifically for their description of base modifications, their synthesis, their use, and their incorporation into oligonucleotides and nucleic acids.

Nucleotide analogs can also include modifications of the sugar moiety. Modifications to the sugar moiety would include natural modifications of the ribose and deoxyribose as well as synthetic modifications. Sugar modifications include but are not limited to the following modifications at the 2′ position: OH; F; O—, S—, or N-alkyl; O—, S—, or N-alkenyl; O—, S- or N-alkynyl; or O-alkyl-O-alkyl, wherein the alkyl, alkenyl and alkynyl may be substituted or unsubstituted C1 to C10, alkyl or C2 to C10 alkenyl and alkynyl. 2′ sugar modifications also include but are not limited to —O[(CH₂)n O]m CH₃, —O(CH₂)n OCH₃, —O(CH₂)n NH₂, —O(CH₂)n CH₃, —O(CH₂)n —ONH₂, and —O(CH₂)nON[(CH₂)n CH₃)]₂, where n and m are from 1 to about 10.

Other modifications at the 2′ position include but are not limited to: C1 to C10 lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH₂ and S. Nucleotide sugar analogs may also have sugar mimetics such as cyclobutyl moieties in place of the pentofuranosyl sugar. There are numerous United States patents that teach the preparation of such modified sugar structures such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety, and specifically for their description of modified sugar structures, their synthesis, their use, and their incorporation into nucleotides, oligonucleotides and nucleic acids.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkages between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference its entirety, and specifically for their description of modified phosphates, their synthesis, their use, and their incorporation into nucleotides, oligonucleotides and nucleic acids.

It is understood that nucleotide analogs need only contain a single modification, but may also contain multiple modifications within one of the moieties or between different moieties.

Nucleotide substitutes are molecules having similar functional properties to nucleotides, but which do not contain a phosphate moiety, such as peptide nucleic acid (PNA). Nucleotide substitutes are molecules that will recognize and hybridize to (base pair to) complementary nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference its entirety, and specifically for their description of phosphate replacements, their synthesis, their use, and their incorporation into nucleotides, oligonucleotides and nucleic acids.

It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. (See also Nielsen et al., Science 254:1497-1500 (1991)).

Oligonucleotides can be comprised of nucleotides and can be made up of different types of nucleotides or the same type of nucleotides. For example, one or more of the nucleotides in an oligonucleotide can be ribonucleotides, 2′-O-methyl ribonucleotides, or a mixture of ribonucleotides and 2′-O-methyl ribonucleotides; about 10% to about 50% of the nucleotides can be ribonucleotides, 2′-O-methyl ribonucleotides, or a mixture of ribonucleotides and 2′-O-methyl ribonucleotides; about 50% or more of the nucleotides can be ribonucleotides, 2′-O-methyl ribonucleotides, or a mixture of ribonucleotides and 2′-O-methyl ribonucleotides; or all of the nucleotides are ribonucleotides, 2′-O-methyl ribonucleotides, or a mixture of ribonucleotides and 2′-O-methyl ribonucleotides. Such oligonucleotides can be referred to as chimeric oligonucleotides.

T. Nucleic Acid Libraries

The disclosed method can be used to produce replicated strands that serve as a nucleic acid library of a nucleic acid sample. Such a nucleic acid library can be used for any purpose, including, for example, detection of sequences, production of probes, production of nucleic acid arrays or chips, and comparison with nucleic acids in other nucleic acid libraries. Similarly prepared nucleic acid libraries of other nucleic acid samples to allow convenient detection of differences between the samples. The nucleic acid libraries can be used both for detection of related nucleic acid samples and comparison of nucleic acid samples. For example, the presence or identity of specific organisms can be detected by producing a nucleic acid library of the test organism and comparing the resulting nucleic acid library with reference nucleic acid libraries prepared from known organisms. Changes and differences in gene expression patterns can also be detected by preparing nucleic acid libraries of mRNA from different cell samples and comparing the nucleic acid libraries. The replicated strands can also be used to produce a set of probes or primers that is specific for the source of a nucleic acid sample. The replicated strands can also be used as a fingerprint of nucleic acid sequences present in a sample. Nucleic acid libraries can be made up of, or derived from, the mRNA of a sample such that the entire relevant mRNA content of the sample is substantially represented.

Nucleic acid libraries can be stored or archived for later use. For example, replicated strands produced in the disclosed method can be physically stored, either in solution, frozen, or attached or adhered to a solid-state substrate such as an array. Storage in an array is useful for providing an archived probe set derived from the nucleic acids in any sample of interest. As another example, informational content of, or derived from, nucleic acid fingerprints can also be stored. Such information can be stored, for example, in or as computer readable media. Examples of informational content of nucleic acid libraries include nucleic acid sequence information (complete or partial); differential nucleic acid sequence information such as sequences present in one sample but not another; hybridization patterns of replicated strands to, for example, nucleic acid arrays, sets, chips, or other replicated strands. Numerous other data that is or can be derived from nucleic acid libraries and replicated strands produced in the disclosed method can also be collected, used, saved, stored, and/or archived.

Nucleic acid libraries can also contain or be made up of other information derived from the information generated in the disclosed method, and can be combined with information obtained or generated from any other source. The informational nature of nucleic acid libraries produced using the disclosed method lends itself to combination and/or analysis using known bioinformatics systems and methods.

Nucleic acid libraries of nucleic acid samples can be compared to a similar nucleic acid library derived from any other sample to detect similarities and differences in the samples (which is indicative of similarities and differences in the nucleic acids in the samples). For example, a nucleic acid library of a first nucleic acid sample can be compared to a nucleic acid library of a sample from the same type of organism as the first nucleic acid sample, a sample from the same type of tissue as the first nucleic acid sample, a sample from the same organism as the first nucleic acid sample, a sample obtained from the same source but at time different from that of the first nucleic acid sample, a sample from an organism different from that of the first nucleic acid sample, a sample from a type of tissue different from that of the first nucleic acid sample, a sample from a strain of organism different from that of the first nucleic acid sample, a sample from a species of organism different from that of the first nucleic acid sample, or a sample from a type of organism different from that of the first nucleic acid sample.

The same type of tissue is tissue of the same type such as liver tissue, muscle tissue, or skin (which may be from the same or a different organism or type of organism). The same organism refers to the same individual, animal, or cell. For example, two samples taken from a patient are from the same organism. The same source is similar but broader, referring to samples from, for example, the same organism, the same tissue from the same organism, the same DNA molecule, or the same DNA library. Samples from the same source that are to be compared can be collected at different times (thus allowing for potential changes over time to be detected). This is especially useful when the effect of a treatment or change in condition is to be assessed. Samples from the same source that have undergone different treatments can also be collected and compared using the disclosed method. A different organism refers to a different individual organism, such as a different patient, a different individual animal. Different organism includes a different organism of the same type or organisms of different types. A different type of organism refers to organisms of different types such as a dog and cat, a human and a mouse, or E. coli and Salmonella. A different type of tissue refers to tissues of different types such as liver and kidney, or skin and brain. A different strain or species of organism refers to organisms differing in their species or strain designation as those terms are understood in the art.

U. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for real-time detection of rolling circle amplification products, the kit comprising one or more rolling circle replication primers and one or more fluorescent change probes. The kits also can contain DNA polymerase, amplification target circles, nucleotides, buffers, ligase, open circle probes, linkers, circularization sequences, or a combination.

V. Mixtures

Disclosed are mixtures formed by performing or preparing to perform the disclosed method. For example, disclosed are mixtures comprising one or more amplification target circles, one or more rolling circle replication primers, and one or more fluorescent change probes; tandem sequence DNA, one or more amplification target circles, one or more rolling circle replication primers, and one or more fluorescent change probes; DNA polymerase, one or more amplification target circles, one or more rolling circle replication primers, and one or more fluorescent change probes; DNA polymerase, tandem sequence DNA, one or more amplification target circles, one or more rolling circle replication primers, and one or more fluorescent change probes; secondary tandem sequence DNA, one or more amplification target circles, one or more rolling circle replication primers, and one or more fluorescent change probes; tandem sequence DNA, secondary tandem sequence DNA, one or more amplification target circles, one or more rolling circle replication primers, and one or more fluorescent change probes; DNA polymerase, tandem sequence DNA, secondary tandem sequence DNA, one or more amplification target circles, one or more rolling circle replication primers, and one or more fluorescent change probes; one or more amplification target circles and one or more fluorescent change rolling circle replication primers; tandem sequence DNA, one or more amplification target circles, and one or more fluorescent change rolling circle replication primers; DNA polymerase, one or more amplification target circles, and one or more fluorescent change rolling circle replication primers; DNA polymerase, tandem sequence DNA, one or more amplification target circles, and one or more fluorescent change rolling circle replication primers; secondary tandem sequence DNA, one or more amplification target circles, and one or more fluorescent change rolling circle replication primers; tandem sequence DNA, secondary tandem sequence DNA, one or more amplification target circles, and one or more fluorescent change rolling circle replication primers; or DNA polymerase, tandem sequence DNA, secondary tandem sequence DNA, one or more amplification target circles, and one or more fluorescent change rolling circle replication primers.

Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

W. Systems

Disclosed are systems useful for performing, or aiding in the performance of, the disclosed method. Systems generally comprise combinations of articles of manufacture such as structures, machines, devices, and the like, and compositions, compounds, materials, and the like. Such combinations that are disclosed or that are apparent from the disclosure are contemplated. For example, disclosed and contemplated are systems comprising solid supports and rolling circle replication primers, amplification target circles, fluorescent change probes, or a combination.

X. Data Structures and Computer Control

Disclosed are data structures used in, generated by, or generated from, the disclosed method. Data structures generally are any form of data, information, and/or objects collected, organized, stored, and/or embodied in a composition or medium. A nucleic acid library stored in electronic form, such as in RAM or on a storage disk, is a type of data structure.

The disclosed method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

### Uses

The disclosed method and compositions are applicable to numerous areas including, but not limited to, analysis of nucleic acids present in cells (for example, analysis of genomic DNA in cells), disease detection, mutation detection, gene discovery, gene mapping (molecular haplotyping), and agricultural research. Particularly useful is whole genome amplification. Other uses include, for example, detection of nucleic acids in cells and on genomic DNA arrays; molecular haplotyping; mutation detection; detection of inherited diseases such as cystic fibrosis, muscular dystrophy, diabetes, hemophilia, sickle cell anemia; assessment of predisposition for cancers such as prostate cancer, breast cancer, lung cancer, colon cancer, ovarian cancer, testicular cancer, pancreatic cancer.

### Method

Disclosed is a method for real-time detection of rolling circle amplification products. Real-time detection is detection that takes place during the amplification reaction or operation. Generally, such detection can be accomplished by detecting amplification product at one or more discrete times during amplification, continuously during all or one or more portions of the amplification, or a combination of discrete times and continuous detection. Real-time detection can be aided by the use of labels or moieties that embody or produce a detectable signal that can be detected without disrupting the amplification reaction or operation. Fluorescent labels are an example of useful labels for real-time detection. A particularly useful means of obtaining real-time detection is the use of fluorescent change probes and/or primers in the amplification operation. With suitably designed fluorescent change probes and primers, fluorescent signals can be generated as amplification proceeds. In most such cases, the fluorescent signals will be in proportion to the amount of amplification product and/or amount of target sequence or target molecule.

In some forms, the disclosed method involves rolling circle amplification and real-time detection of amplification products where amplification includes multiply-primed rolling circle amplification (MPRCA). Rolling circle amplification (RCA) refers to nucleic acid amplification reactions involving replication of a circular nucleic acid template to form a long strand with tandem repeats of the sequence complementary to the circular template. Rolling circle replication can be primed at one or more sites on the circular template. Multiply-primed RCA refers to RCA where replication is primed at a plurality of sites on the circular template. Multiply-primed RCA increases the sensitivity of singly-primed rolling circle amplification. Rolling circle amplification refers both to rolling circle replication and to processes involving both rolling circle replication and additional forms of amplification (such as replication of tandem sequence DNA).

Multiply-primed RCA can be performed using a single primer (which hybridizes to multiple sites on the amplification target circle) or multiple primers (each of which can hybridize to a single site on the amplification target circle or multiple sites on the amplification target circle). Multiple priming (as occurs in MPRCA) can increase the yield of amplified product from RCA. Primers anneal to multiple locations on the circular template and a product of extension by polymerase is initiated from each location. In this way, multiple extensions are achieved simultaneously from a single amplification target circle.

Multiple priming can be achieved in several different ways. For example, two or more specific primers that anneal to different sequences on the circular template can be used, one or more specific primers that each anneals to a sequence repeated at two or more separate locations on the circular template can be used, a combination of primers that each anneal to a different sequence on the circular template or to a sequence repeated at two or more separate locations on the circular templates can be used, one or more random or degenerate primers, which can anneal to many locations on the circle, can be used, or a combination of such primers can be used.

A. Rolling Circle Amplification

The disclosed method involves rolling circle amplification. Rolling circle amplification refers to nucleic acid amplification reactions where a circular nucleic acid template is replicated in a single long strand with tandem repeats of the sequence of the circular template. This first, directly produced tandem repeat strand is referred to as tandem sequence DNA (TS-DNA) and its production is referred to as rolling circle replication. Rolling circle amplification refers both to rolling circle replication and to processes involving both rolling circle replication and additional forms of amplification. For example, tandem sequence DNA can be replicated to form complementary strands referred to a secondary tandem sequence DNA. Secondary tandem sequence DNA can, in turn, be replicated, and so on. Tandem sequence DNA can also be transcribed. Rolling circle amplification involving production of only the first tandem sequence DNA (that is, the replicated strand produced by rolling circle replication) can be referred to as of linear rolling circle amplification (where “linear” refers to the general amplification kinetics of the amplification).

When rolling circle amplification is involved the rolling circle replication primer and the rolling circle template must be associated together. This typically can occur through mixing one or more amplification target circles with the rolling circle replication primers under conditions that promote association of the rolling circle replication primers with the amplification target circles. To get replication of the amplification target circles the amplification target circle and the rolling circle replication primer typically are incubated under conditions that promote replication of the amplification target circles, wherein replication of the amplification target circles results in the formation of tandem sequence DNA. There are numerous variations of rolling circle amplification that can be used in the disclosed methods. Some useful variations of rolling circle amplification are described in, for example, U.S. Pat. No. 5,563,912, U.S. Pat. No. 6,143,495, and U.S. Pat. No. 6,316,229. In some embodiments the tandem sequence DNA can itself be replicated or otherwise amplified.

In the disclosed method, the amplification or amplification products are detected during the amplification reaction or operation. That is, the progress of amplification or amplification products are detected in real-time. This can be accomplished in any suitable manner, but preferably involves the use of one or more fluorescent change probes and/or one or more fluorescent change primers.

B. Amplification Operation

The basic form of amplification operation is rolling circle replication of a circular DNA molecule (that is, a circularized open circle probe or an amplification target circle). Rolling circle amplification generally requires use of one or more rolling circle replication primers, which are complementary to the primer complement portions of the ATC, and a rolling circle DNA polymerase. The DNA polymerase catalyzes primer extension and strand displacement in a processive rolling circle polymerization reaction that proceeds as long as desired, generating a large DNA molecule that contains a large number of tandem copies of a sequence complementary to the amplification target circle. Some forms of the disclosed method use rolling circle replication primers and secondary DNA strand displacement primers in the amplification reaction.

In multiply-primed RCA, one or more rolling circle replication primers anneal at various places on an amplification target circle to generate multiple replication forks. As each strand grows, the DNA polymerase encounters an adjacent replicating strand and displaces it from the amplification target circle. The result is multiple copies of each circle being produced simultaneously. Multiply-primed RCA can be performed using a single primer (which hybridizes to multiple sites on the amplification target circle) or multiple primers (each of which can hybridize to a single site on the amplification target circle or multiple sites on the amplification target circle). Multiple priming (as occurs in MPRCA) can increase the yield of amplified product from RCA. Primers anneal to multiple locations on the circular template and a product of extension by polymerase is initiated from each location. In this way, multiple extensions are achieved simultaneously from a single amplification target circle.

The amplification operation also involves detection of amplification during the amplification operation (that is, real-time detection). This can be accomplished in any suitable manner. A particularly useful means of obtaining real-time detection is the use of fluorescent change probes and/or primers in the amplification operation. With suitably designed fluorescent change probes and primers, fluorescent signals can be generated as amplification proceeds. In most such cases, the fluorescent signals will be in proportion to the amount of amplification product and/or amount of target sequence or target molecule.

In the disclosed method, detection generally will be during rolling circle amplification and preferably is accomplished through the use of fluorescent changes probes and/or primers. For example, rolling circle replication primers and/or secondary DNA strand displacement primers can be fluorescent change primers. Alternatively or in addition, detection probes that are fluorescent change probes can be used.

As well as rolling circle replication, the amplification operation can include additional nucleic acid replication or amplification processes. For example, TS-DNA can itself be replicated to form secondary TS-DNA. This process is referred to as secondary DNA strand displacement. The combination of rolling circle replication and secondary DNA strand displacement is referred to as linear rolling circle amplification (LRCA). The secondary TS-DNA can itself be replicated to form tertiary TS-DNA in a process referred to as tertiary DNA strand displacement. Secondary and tertiary DNA strand displacement can be performed sequentially or simultaneously. When performed simultaneously, the result is strand displacement cascade amplification. The combination of rolling circle replication and strand displacement cascade amplification is referred to as exponential rolling circle amplification (ERCA). Secondary TS-DNA, tertiary TS-DNA, or both can-be amplified by transcription. Exponential rolling circle amplification is a preferred form of amplification operation.

After RCA, a round of LM-RCA can be performed on the TS-DNA produced in the first RCA. This new round of LM-RCA can be performed with a new open circle probe, referred to as a secondary open circle probe, having target probe portions complementary to a target sequence in the TS-DNA produced in the first round. When such new rounds of LM-RCA are performed, the amplification is referred to as nested LM-RCA. Nested LM-RCA can also be performed on ligated OCPs or ATCs that have not been amplified. In this case, LM-RCA can be carried out using either ATCs or target-dependent ligated OCPs. This is especially useful for in situ detection. For in situ detection, the first, unamplified OCP, which is topologically locked to its target sequence, can be subjected to nested LM-RCA. By not amplifying the first OCP, it can remain hybridized to the target sequence while LM-RCA amplifies a secondary OCP topologically locked to the first OCP. Nested LM-RCA is described in U.S. Pat. No. 6,143,495.

When an open circle probe is used to form the amplification target circle, the amplification target circle can be formed by target-mediated ligation. Where OCPs are used, the tandem sequence DNA consists of alternating target sequence and spacer sequence. Note that the spacer sequence of the TS-DNA is the complement of the sequence between the left target probe and the right target probe in the original open circle probe.

1. DNA Strand Displacement

DNA strand displacement is one way to amplify TS-DNA. Secondary DNA strand displacement is accomplished by hybridizing secondary DNA strand displacement primers to TS-DNA and allowing a DNA polymerase to synthesize DNA from these primed sites (see FIG. 11 in U.S. Pat. No. 6,143,495). Because a complement of the secondary DNA strand displacement primer occurs in each repeat of the TS-DNA, secondary DNA strand displacement can result in a high level of amplification. The product of secondary DNA strand displacement is referred to as secondary tandem sequence DNA or TS-DNA-2. Secondary DNA strand displacement can be accomplished by performing RCA to produce TS-DNA, mixing secondary DNA strand displacement primer with the TS-DNA, and incubating under conditions promoting replication of the tandem sequence DNA.

Secondary DNA strand displacement can also be carried out simultaneously with rolling circle replication. This is accomplished by mixing secondary DNA strand displacement primer with the reaction prior to rolling circle replication. As a secondary DNA strand displacement primer is elongated, the DNA polymerase will run into the 5′ end of the next hybridized secondary DNA strand displacement molecule and will displace its 5′ end. In this fashion a tandem queue of elongating DNA polymerases is formed on the TS-DNA template. As long as the rolling circle reaction continues, new secondary DNA strand displacement primers and new DNA polymerases are added to TS-DNA at the growing end of the rolling circle. The generation of TS-DNA-2 and its release into solution by strand displacement is shown diagrammatically in FIG. 11 in U.S. Pat. No. 6,143,495. For simultaneous rolling circle replication and secondary DNA strand displacement, it is preferred that the rolling circle DNA polymerase be used for both replications. This allows optimum conditions to be used and results in displacement of other strands being synthesized downstream. Secondary DNA strand displacement can follow any DNA replication operation, such as RCA, LM-RCA or nested LM-RCA.

Generally, secondary DNA strand displacement can be performed by, simultaneous with or following RCA, mixing a secondary DNA strand displacement primer with the reaction mixture and incubating under conditions that promote both hybridization between the tandem sequence DNA and the secondary DNA strand displacement primer, and replication of the tandem sequence DNA, where replication of the tandem sequence DNA results in the formation of secondary tandem sequence DNA.

When secondary DNA strand displacement is carried out in the presence of a tertiary DNA strand displacement primer (or an equivalent primer), an exponential amplification of TS-DNA sequences takes place. This special and preferred mode of DNA strand displacement is referred to as strand displacement cascade amplification (SDCA) and is a form of exponential rolling circle amplification (ERCA). In SDCA, a secondary DNA strand displacement primer primes replication of TS-DNA to form TS-DNA-2, as described above. The tertiary DNA strand displacement primer strand can then hybridize to, and prime replication of, TS-DNA-2 to form TS-DNA-3. Strand displacement of TS-DNA-3 by the adjacent, growing TS-DNA-3 strands makes TS-DNA-3 available for hybridization with secondary DNA strand displacement primer. This results in another round of replication resulting in TS-DNA-4 (which is equivalent to TS-DNA-2). TS-DNA-4, in turn, becomes a template for DNA replication primed by tertiary DNA strand displacement primer. The cascade continues this manner until the reaction stops or reagents become limiting. This reaction amplifies DNA at an almost exponential rate. In a useful mode of SDCA, the rolling circle replication primers serve as the tertiary DNA strand displacement primer, thus eliminating the need for a separate primer. The additional forms of tandem sequence DNA beyond secondary tandem sequence DNA are collectively referred to herein as higher order tandem sequence DNA. Higher order tandem sequence DNA encompasses TS-DNA-3, TS-DNA-4, and any other tandem sequence DNA produced from replication of secondary tandem sequence DNA or the products of such replication.

For this mode, the rolling circle replication primer should be used at a concentration sufficiently high to obtain rapid priming on the growing TS-DNA-2 strands. To optimize the efficiency of SDCA, it is preferred that a sufficient concentration of secondary DNA strand displacement primer and tertiary DNA strand displacement primer be used to obtain sufficiently rapid priming of the growing TS-DNA strand to out compete TS-DNA for binding to its complementary TS-DNA. Optimization of primer concentrations are described in U.S. Pat. No. 6,143,495 and can be aided by analysis of hybridization kinetics (Young and Anderson, “Quantitative analysis of solution hybridization” in Nucleic Acid Hybridization: A Practical Approach (IRL Press, 1985) pages 47-71).

Generally, strand displacement cascade amplification can be performed by, simultaneous with, or following, RCA, mixing a secondary DNA strand displacement primer and a tertiary DNA strand displacement primer with the reaction mixture and incubating under conditions that promote hybridization between the tandem sequence DNA and the secondary DNA strand displacement primer, replication of the tandem sequence DNA—where replication of the tandem sequence DNA results in the formation of secondary tandem sequence DNA—hybridization between the secondary tandem sequence DNA and the tertiary DNA strand displacement primer, and replication of secondary tandem sequence DNA—where replication of the secondary tandem sequence DNA results in formation of tertiary tandem sequence DNA (TS-DNA-3).

Secondary and tertiary DNA strand displacement can also be carried out sequentially. Following a first round of secondary DNA strand displacement, a tertiary DNA strand displacement primer can be mixed with the secondary tandem sequence DNA and incubated under conditions that promote hybridization between the secondary tandem sequence DNA and the tertiary DNA strand displacement primer, and replication of secondary tandem sequence DNA, where replication of the secondary tandem sequence DNA results in formation of tertiary tandem sequence DNA (TS-DNA-3). This round of strand displacement replication can be referred to as tertiary DNA strand displacement. However, all rounds of strand displacement replication following rolling circle replication can also be referred to collectively as DNA strand displacement or secondary DNA strand displacement.

A modified form of secondary DNA strand displacement results in amplification of TS-DNA and is referred to as opposite strand amplification (OSA). OSA is the same as secondary DNA strand displacement except that a special form of rolling circle replication primer is used that prevents it from hybridizing to TS-DNA-2. Opposite strand amplification is described in U.S. Pat. No. 6,143,495.

The DNA generated by DNA strand displacement can be labeled and/or detected using the same labels, labeling methods, and detection methods described for use with TS-DNA. In the disclosed method, detection generally will be during DNA strand displacement and preferably is accomplished through the use of fluorescent changes probes and/or primers. For example, secondary DNA strand displacement primers and/or tertiary DNA strand displacement primers can be fluorescent change primers. Alternatively or in addition, detection probes that are fluorescent change probes can be used.

2. Geometric Rolling Circle Amplification

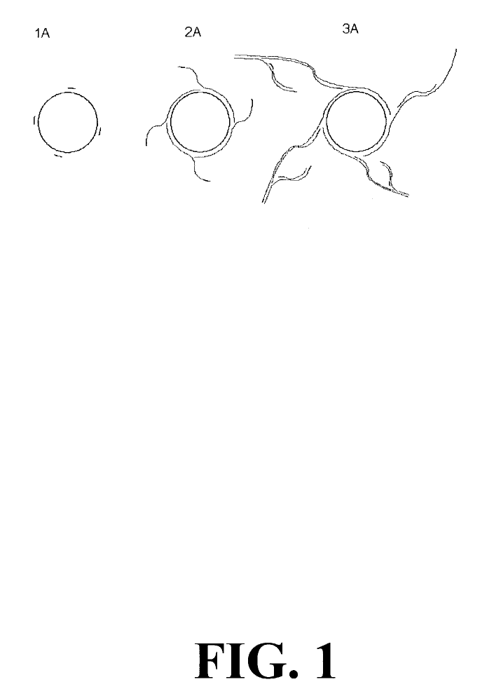

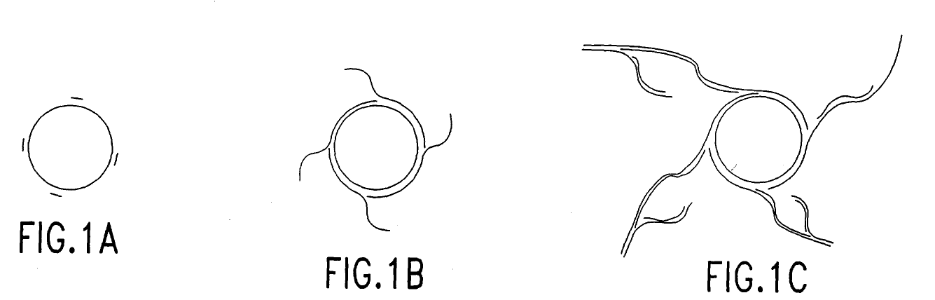

RCA reactions can be carried out with either linear or geometric kinetics (Lizardi et al., 1998). Linear rolling circle amplification generally follows linear kinetics. Two useful forms of RCA with geometric kinetics are exponential multiply-primed rolling circle amplification (EMPRCA) and exponential rolling circle amplification (ERCA). In exponential multiply-primed RCA, one or more rolling circle replication primers anneal at various places on the amplification target circle to generate multiple replication forks (FIG. 1A). As each strand grows, the DNA polymerase encounters an adjacent replicating strand and displaces it from the amplification target circle (FIG. 1B). The result is multiple copies of each circle being produced simultaneously. The replicated strands are referred to as tandem sequence DNA (TS-DNA). As each TS-DNA strand is displaced from the circular template, secondary DNA strand displacement primers can anneal to, and prime replication of, the TS-DNA (FIG. 1C). Replication of the TS-DNA forms complementary strands referred to as secondary tandem sequence DNA or TS-DNA-2. As a secondary TS-DNA strand is elongated, the DNA polymerase will run into the 5′ end of the next growing strand of secondary TS-DNA and will displace its 5′ end. In this fashion a tandem queue of elongating DNA polymerases is formed on the TS-DNA template. As long as the rolling circle reaction continues, new primers and new DNA polymerases are added to TS-DNA at the growing end of the rolling circle.

Random or degenerate primers can be used to perform multiply-primed RCA. Such random or degenerate primers will anneal to multiple sites on the amplification target circle (resulting in production of tandem sequence DNA), as well as to multiple sites on the tandem sequence DNA (resulting in production of secondary tandem sequence DNA). The random primers can then hybridize to, and prime replication of, TS-DNA-2 to form TS-DNA-3 (which is equivalent to the original TS-DNA). Strand displacement of TS-DNA-3 by the adjacent, growing TS-DNA-3 strands makes TS-DNA-3 available for hybridization with the primers. This can result in another round of replication resulting in TS-DNA-4 (which is equivalent to TS-DNA-2). TS-DNA-4, in turn, becomes a template for DNA replication primed by random primers. The cascade continues this manner until the reaction stops or reagents become limiting. Multiply-primed RCA is particularly useful for amplifying larger circular templates such as amplification target circles that are, or are derived from or include, nucleic acid molecules of interest. Multiply-primed RCA is described in Dean et al., Rapid Amplification of Plasmid and Phage DNA Using Phi29 DNA Polymerase and Multiply-Primed Rolling Circle Amplification, Genome Research 11: 1095-1099 (2001).

Exponential multiply-primed RCA also can be achieved using specific rolling circle replication primers, secondary DNA strand displacement primers and tertiary DNA strand displacement primers. In this form of the disclosed method, rolling circle replication is primed from multiple specific primer complement portions of the circular template. As the strand grows, the DNA polymerase encounters 5′ end of the strand and displaces it from the circular template. A secondary DNA strand displacement primer primes replication of TS-DNA to form a complementary strand referred to as secondary tandem sequence DNA or TS-DNA-2. As a secondary DNA strand displacement primer is elongated, the DNA polymerase will run into the 5′ end of the next hybridized secondary DNA strand displacement molecule and will displace its 5′ end. In this fashion a tandem queue of elongating DNA polymerases is formed on the TS-DNA template. As long as the rolling circle reaction continues, new secondary DNA strand displacement primers and new DNA polymerases are added to TS-DNA at the growing end of the rolling circle. A tertiary DNA strand displacement primer strand (which is complementary to the TS-DNA-2 strand and which can be the rolling circle replication primer) can then hybridize to, and prime replication of, TS-DNA-2 to form TS-DNA-3 (which is equivalent to the original TS-DNA). Strand displacement of TS-DNA-3 by the adjacent, growing TS-DNA-3 strands makes TS-DNA-3 available for hybridization with secondary DNA strand displacement primer. This results in another round of replication resulting in TS-DNA-4 (which is equivalent to TS-DNA-2). TS-DNA-4, in turn, becomes a template for DNA replication primed by tertiary DNA strand displacement primer. The cascade continues this manner until the reaction stops or reagents become limiting. In one mode of ERCA, the rolling circle replication primer serves as the tertiary DNA strand displacement primer, thus eliminating the need for a separate primer. Exponential RCA and other useful forms of RCA are described in U.S. Pat. No. 5,854,033, and U.S. Pat. No. 6,143,495.

C. Detection of Amplification Products

Products of the amplification operation can be detected using any nucleic acid detection technique. For real-time detection, the amplification products and the progress of amplification are detected during the amplification operation. Real-time detection is usefully accomplished using one or more or one or a combination of fluorescent change probes and fluorescent change primers. Other detection techniques can be used, either alone or in combination with real-timer detection and/or detection involving fluorescent change probes and primers. Many techniques are known for detecting nucleic acids. The nucleotide sequence of the amplified sequences also can be determined using any suitable technique.

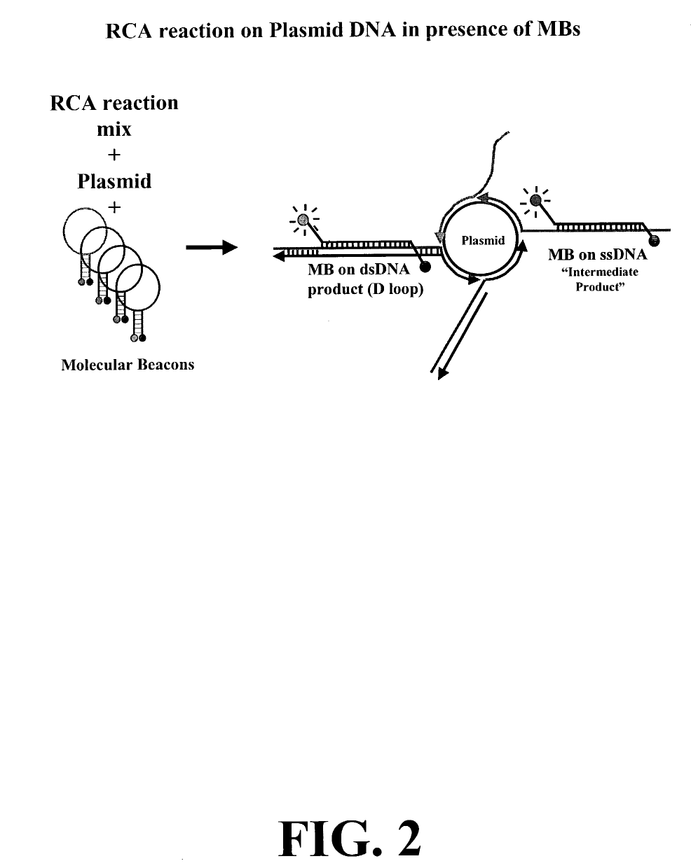

FIG. 2 shows a typical real-time detection scheme for a multiply-primed rolling circle amplification. This illustration involves the use of nuclease-resistant random hexamer primers for multiply-primed RCA and of molecular beacon probes as fluorescent change probes for real-time detection.

1. Primary Labeling

Primary labeling consists of incorporating labeled moieties, such as fluorescent nucleotides, biotinylated nucleotides, digoxygenin-containing nucleotides, or bromodeoxyuridine, during rolling circle replication in RCA, or during transcription in RCT. For example, fluorescent labels can be incorporated into replicated nucleic acid by using fluorescently labeled primers, such as fluorescent change rolling circle replication primers. In another example, one can incorporate cyanine dye UTP analogs (Yu et al. (1994)) at a frequency of 4 analogs for every 100 nucleotides. A preferred method for detecting nucleic acid amplified in situ is to label the DNA during amplification with BrdUrd, followed by binding of the incorporated BUDR with a biotinylated anti-BUDR antibody (Zymed Labs, San Francisco, Calif.), followed by binding of the biotin moieties with Streptavidin-Peroxidase (Life Sciences, Inc.), and finally development of fluorescence with Fluorescein-tyramide (DuPont de Nemours & Co., Medical Products Dept.).

A useful form of primary labeling is the use of fluorescent change primers in the amplification operation. Fluorescent change primers exhibit a change in fluorescence intensity or wavelength based on a change in the form or conformation of the primer and the amplified nucleic acid. Stem quenched primers are primers that when not hybridized to a complementary sequence form a stem structure (either an intramolecular stem structure or an intermolecular stem structure) that brings a fluorescent label and a quenching moiety into proximity such that fluorescence from the label is quenched. When the primer binds to a complementary sequence, the stem is disrupted, the quenching moiety is no longer in proximity to the fluorescent label and fluorescence increases. In the disclosed method, stem quenched primers are used as primers for nucleic acid synthesis and thus become incorporated into the synthesized or amplified nucleic acid. Examples of stem quenched primers are peptide nucleic acid quenched primers and hairpin quenched primers.

Peptide nucleic acid quenched primers are primers associated with a peptide nucleic acid quencher or a peptide nucleic acid fluor to form a stem structure. The primer contains a fluorescent label or a quenching moiety and is associated with either a peptide nucleic acid quencher or a peptide nucleic acid fluor, respectively. This puts the fluorescent label in proximity to the quenching moiety. When the primer is replicated, the peptide nucleic acid is displaced, thus allowing the fluorescent label to produce a fluorescent signal.

Hairpin quenched primers are primers that when not hybridized to a complementary sequence form a hairpin structure (and, typically, a loop) that brings a fluorescent label and a quenching moiety into proximity such that fluorescence from the label is quenched. When the primer binds to a complementary sequence, the stem is disrupted, the quenching moiety is no longer in proximity to the fluorescent label and fluorescence increases. Hairpin quenched primers are typically used as primers for nucleic acid synthesis and thus become incorporated into the synthesized or amplified nucleic acid. Examples of hairpin quenched primers are Amplifluor primers and scorpion primers.

Cleavage activated primers are primers where fluorescence is increased by cleavage of the primer. Generally, cleavage activated primers are incorporated into replicated strands and are then subsequently cleaved. Cleavage activated primers can include a fluorescent label and a quenching moiety in proximity such that fluorescence from the label is quenched. When the primer is clipped or digested (typically by the 5′-3′ exonuclease activity of a polymerase during amplification), the quenching moiety is no longer in proximity to the fluorescent label and fluorescence increases. Little et al., Clin. Chem. 45:777-784 (1999), describe the use of cleavage activated primers.

2. Secondary Labeling

Secondary labeling consists of using suitable molecular probes, such as detection probes, to detect the amplified nucleic acids. For example, an amplification target circle may be designed to contain several repeats of a known arbitrary sequence, referred to as detection tags. The detection probes can then be hybridized to these detection tags. The detection probes may be labeled as described above with, for example, an enzyme, fluorescent moieties, or radioactive isotopes. By using three detection tags per amplification target circle, and four fluorescent moieties per each detection probe, one may obtain a total of twelve fluorescent signals for every amplification target circle repeat in the TS-DNA, yielding a total of 12,000 fluorescent moieties for every amplification target circle that is amplified by RCA. Detection probes can interact by hybridization or annealing via normal Watson-Crick base-pairing (or related alternatives) or can interact with double-stranded targets to form a triple helix. Such triplex-forming detection probes can be used in the same manner as other detection probes, such as in the form of fluorescent change probes.

A useful form of secondary labeling is the use of fluorescent change probes and primers in or following the amplification operation. Hairpin quenched probes are probes that when not bound to a target sequence form a hairpin structure (and, typically, a loop) that brings a fluorescent label and a quenching moiety into proximity such that fluorescence from the label is quenched. When the probe binds to a target sequence, the stem is disrupted, the quenching moiety is no longer in proximity to the fluorescent label and fluorescence increases. Examples of hairpin quenched probes are molecular beacons, fluorescent triplex oligos, triplex molecular beacons, triplex FRET probes, and QPNA probes.

Cleavage activated probes are probes where fluorescence is increased by cleavage of the probe. Cleavage activated probes can include a fluorescent label and a quenching moiety in proximity such that fluorescence from the label is quenched. When the probe is clipped or digested (typically by the 5′-3′ exonuclease activity of a polymerase during or following amplification), the quenching moiety is no longer in proximity to the fluorescent label and fluorescence increases. TaqMan probes are an example of cleavage activated probes.

Cleavage quenched probes are probes where fluorescence is decreased or altered by cleavage of the probe. Cleavage quenched probes can include an acceptor fluorescent label and a donor moiety such that, when the acceptor and donor are in proximity, fluorescence resonance energy transfer from the donor to the acceptor causes the acceptor to fluoresce. The probes are thus fluorescent, for example, when hybridized to a target sequence. When the probe is clipped or digested (typically by the 5′-3′ exonuclease activity of a polymerase during or after amplification), the donor moiety is no longer in proximity to the acceptor fluorescent label and fluorescence from the acceptor decreases. If the donor moiety is itself a fluorescent label, it can release energy as fluorescence (typically at a different wavelength than the fluorescence of the acceptor) when not in proximity to an acceptor. The overall effect would then be a reduction of acceptor fluorescence and an increase in donor fluorescence. Donor fluorescence in the case of cleavage quenched probes is equivalent to fluorescence generated by cleavage activated probes with the acceptor being the quenching moiety and the donor being the fluorescent label. Cleavable FRET (fluorescence resonance energy transfer) probes are an example of cleavage quenched probes.

Fluorescent activated probes are probes or pairs of probes where fluorescence is increased or altered by hybridization of the probe to a target sequence. Fluorescent activated probes can include an acceptor fluorescent label and a donor moiety such that, when the acceptor and donor are in proximity (when the probes are hybridized to a target sequence), fluorescence resonance energy transfer from the donor to the acceptor causes the acceptor to fluoresce. Fluorescent activated probes are typically pairs of probes designed to hybridize to adjacent sequences such that the acceptor and donor are brought into proximity. Fluorescent activated probes can also be single probes containing both a donor and acceptor where, when the probe is not hybridized to a target sequence, the donor and acceptor are not in proximity but where the donor and acceptor are brought into proximity when the probe hybridized to a target sequence. This can be accomplished, for example, by placing the donor and acceptor on opposite ends a the probe and placing target complement sequences at each end of the probe where the target complement sequences are complementary to adjacent sequences in a target sequence. If the donor moiety of a fluorescent activated probe is itself a fluorescent label, it can release energy as fluorescence (typically at a different wavelength than the fluorescence of the acceptor) when not in proximity to an acceptor (that is, when the probes are not hybridized to the target sequence). When the probes hybridize to a target sequence, the overall effect would then be a reduction of donor fluorescence and an increase in acceptor fluorescence. FRET probes are an example of fluorescent activated probes. Stem quenched primers (such as peptide nucleic acid quenched primers and hairpin quenched primers) can be used as secondary labels.

3. Multiplexing and Hybridization Array Detection

RCA is easily multiplexed by using sets of different amplification target circles, each amplification target circle being associated with, for example, different target molecules, target sequences, and/or array positions. Each amplification target circle can have a different primer complement portions and/or different detection tag portions corresponding to different rolling circle replication primers and/or different detection probes. Use of different fluorescent labels with different rolling circle replication primers and/or different detection probes allows specific detection of different open circle probes (and thus, of different targets).

For multiplexing, the mixture of amplification target circle(s), rolling circle replication primer(s) and fluorescent change probe(s) in the disclosed method can comprise a plurality of amplification target circles. The fluorescent change probes each can comprise a complementary portion, the amplification target circles each can comprise at least one detection tag portion, and the complementary portion of each of the fluorescent change probes matches the sequence of one or more of the detection tag portions of the amplification target circles. The mixture can comprise a plurality of fluorescent change probes, where the complementary portion of each fluorescent change probe matches the sequence of one or more of the detection tag portions of a different one of the amplification target circles. The mixture can comprise a plurality of fluorescent change probes, where the complementary portion of each fluorescent change probe matches the sequence of one or more of the detection tag portions of one or more of the amplification target circles. The mixture can comprise a plurality of fluorescent change probes, where the complementary portion of each fluorescent change probe matches the sequence of one of the detection tag portions of a different one of the amplification target circles. The mixture can comprise a plurality of fluorescent change probes, where the complementary portion of each fluorescent change probe matches the sequence of a plurality of the detection tag portions of a different one of the amplification target circles. The mixture can comprise a plurality of fluorescent change probes, where the complementary portion of each fluorescent change probe matches the sequence of a plurality of the detection tag portions of one of the amplification target circles. The mixture can comprise a plurality of fluorescent change probes, where the complementary portion of each fluorescent change probe matches the sequence of a plurality of the detection tag portions of a plurality of the amplification target circles. The mixture can comprise a plurality of fluorescent change probes, where the complementary portion of each fluorescent change probe matches the sequence of one of the detection tag portions of a plurality of the amplification target circles. The mixture can comprise a plurality of fluorescent change probes, where the complementary portion of each fluorescent change probe matches the sequence of one of the detection tag portions of one of the amplification target circles.

RCA can also be multiplexed by using sets of different open circle probes, each open circle probe carrying different target probe sequences designed for binding to unique targets and each open circle probe having a different primer complement portions and/or different detection tag portions corresponding to different rolling circle replication primers and/or different detection probes. Only those open circle probes that are able to find their targets will give rise to TS-DNA. Use of different fluorescent labels with different rolling circle replication primers and/or different detection probes allows specific detection of different open circle probes (and thus, of different targets).

The TS-DNA molecules generated by RCA are of high molecular weight and low complexity; the complexity being the length of the amplification target circle. There several ways to capture a given TS-DNA to a fixed position in a solid-state detector. One is to include within the amplification target circles a unique address tag sequence for each unique amplification target circle. TS-DNA generated from a given amplification target circle will then contain sequences corresponding to a specific address tag sequence. Another way to capture TS-DNA when open circle probes are used is to use the target sequence present on the TS-DNA as the address tag.

4. Detecting Multiple Amplification Target Circles

Multiplex RCA assays are useful for detecting multiple amplification target circles. A single RCA assay can be used to detect the presence of one or more members of a group of any number of amplification target circles (and, thus, any number of corresponding target sequences or target molecules). By associating different amplification target circles with different target molecules (using reporter binding agents specific for the target molecules), each different target molecule can be detected by differential detection of the various amplification target circles. This can be accomplished, for example, by designing an amplification target circle for each target molecule, where the detection tag portions and/or the primer complement portions of each amplification target circle are different. Amplification of the different ATCs can be detected based on different primer complement portion sequences by using, for example, rolling circle replication primers that are fluorescent change primers. Alternatively, the different amplification target circles can be detected based on different detection tag sequences by using, for example, detection probes that are fluorescent change probes. In this case, the primer portions of all the amplification target circles can be the same. Use of different detection tag sequences and different detection probes also allows differential detection of amplification target circles even when random or degenerate primers are used for multiply-primed RCA. Different detection probes can be used to detect the various TS-DNAs (each having specific detection tag sequences).

By associating different target sequences or different amplification target circles with different target molecules, such as proteins (using reporter binding agents specific for the proteins of interest), each different target molecule can be detected by differential detection of the various target sequences. This can be accomplished, for example, by designing an open circle probe (and associated gap oligonucleotides, if desired) for each target sequence in the group, where the target probe portions and the detection primer complement portions of each open circle probe are different but the sequence of the common primer complement portions and secondary DNA strand displacement matching portions of all the open circle probes are the same. All of the open circle probes are placed in the same OCP-target sample mixture, and the same primers are used to amplify. For each target sequence present in the assay (those associated with proteins present in the target sample, for example), the OCP for that target will be ligated into a circle and the circle will be amplified to form TS-DNA. Since the detection primer complement portions are different, amplification of the different OCPs can be detected (using, for example, rolling circle replication primers that are fluorescent change primers). Alternatively, the open circle probes can each target a different target sequence in the group, where the target probe portions and the sequence of the detection tag portions of each open circle probe are different but the sequence of the primer complement portions of all the open circle probes are the same. Different detection probes are used to detect the various TS-DNAs (each having specific detection tag sequences). For each target sequence present in the assay (those associated with proteins present in the target sample, for example), the OCP for that target will be ligated into a circle and the circle will be amplified to form TS-DNA. Since the detection tags on TS-DNA resulting from amplification of the OCPs are the different, TS-DNA resulting from ligation each OCP can be detected individually in that assay.

5. Combinatorial Multicolor Coding

One form of multiplex detection involves the use of a combination of labels that either fluoresce at different wavelengths or are colored differently. One of the advantages of fluorescence for the detection of hybridization probes is that several targets can be visualized simultaneously in the same sample. Using a combinatorial strategy, many more targets can be discriminated than the number of spectrally resolvable fluorophores. Combinatorial labeling provides the simplest way to label probes in a multiplex fashion since a probe fluor is either completely absent (−) or present in unit amounts (+); image analysis is thus more amenable to automation, and a number of experimental artifacts, such as differential photobleaching of the fluors and the effects of changing excitation source power spectrum, are avoided. Combinatorial labeling can be used with fluorescent change probes and primers.

The combinations of labels establish a code for identifying different detection probes and, by extension, different target molecules to which those detection probes are associated with. This labeling scheme is referred to as Combinatorial Multicolor Coding (CMC). Such coding is described by Speicher et al., Nature Genetics 12:368-375 (1996). Use of CMC in connection with rolling circle amplification is described in U.S. Pat. No. 6,143,495. Any number of labels, which when combined can be separately detected, can be used for combinatorial multicolor coding. It is preferred that 2, 3, 4, 5, or 6 labels be used in combination. It is most preferred that 6 labels be used. The number of labels used establishes the number of unique label combinations that can be formed according to the formula 2^{N}−1, where N is the number of labels. According to this formula, 2 labels forms three label combinations, 3 labels forms seven label combinations, 4 labels forms 15 label combinations, 5 labels form 31 label combinations, and 6 labels forms 63 label combinations.

For combinatorial multicolor coding, a group of different detection probes are used as a set. Each type of detection probe in the set is labeled with a specific and unique combination of fluorescent labels. For those detection probes assigned multiple labels, the labeling can be accomplished by labeling each detection probe molecule with all of the required labels. Alternatively, pools of detection probes of a given type can each be labeled with one of the required labels. By combining the pools, the detection probes will, as a group, contain the combination of labels required for that type of detection probe. Where each detection probe is labeled with a single label, label combinations can also be generated by using OCPs or ATCs with coded combinations of detection tags complementary to the different detection probes. In this scheme, the OCPs or ATCs will contain a combination of detection tags representing the combination of labels required for a specific label code. Further illustrations are described in U.S. Pat. No. 6,143,495. Use of pools of detection probes each probe with a single label is preferred when fluorescent change probes are used.

Speicher et al. describes a set of fluors and corresponding optical filters spaced across the spectral interval 350-770 nm that give a high degree of discrimination between all possible fluor pairs. This fluor set, which is preferred for combinatorial multicolor coding, consists of 4′-6-diamidino-2-phenylinodole (DAPI), fluorescein (FITC), and the cyanine dyes Cy3, Cy3.5, Cy5, Cy5.5 and Cy7. Any subset of this preferred set can also be used where fewer combinations are required. The absorption and emission maxima, respectively, for these fluors are: DAPI (350 nm; 456 nm), FITC (490 nm; 520 nm), Cy3 (554 nm; 568 nm), Cy3.5 (581 nm; 588 nm), Cy5 (652 nm; 672 nm), Cy5.5 (682 nm; 703 nm) and Cy7 (755 nm; 778 nm). The excitation and emission spectra, extinction coefficients and quantum yield of these fluors are described by Ernst et al., Cytometry 10:3-10 (1989), Mujumdar et al., Cytometry 10:11-19 (1989), Yu, Nucleic Acids Res. 22:3226-3232 (1994), and Waggoner, Meth. Enzymology 246:362-373 (1995). These fluors can all be excited with a 75W Xenon arc.

To attain selectivity, filters with bandwidths in the range of 5 to 16 nm are preferred. To increase signal discrimination, the fluors can be both excited and detected at wavelengths far from their spectral maxima. Emission bandwidths can be made as wide as possible. For low-noise detectors, such as cooled CCD cameras, restricting the excitation bandwidth has little effect on attainable signal to noise ratios. A list of preferred filters for use with the preferred fluor set is listed in Table 1 of Speicher et al. It is important to prevent infra-red light emitted by the arc lamp from reaching the detector; CCD chips are extremely sensitive in this region. For this purpose, appropriate IR blocking filters can be inserted in the image path immediately in front of the CCD window to minimize loss of image quality. Image analysis software can then be used to count and analyze the spectral signatures of fluorescent dots.

D. Ligation Operation

If an open circle probe is used in or with the disclosed method, a ligation operation will be used to circularize the open circle probe (and thus form an amplification target circle). An open circle probe, optionally in the presence of one or more gap oligonucleotides, can be incubated with a sample containing nucleic acids, under suitable hybridization conditions, and then ligated to form a covalently closed circle. The ligated open circle probe is a form of amplification target circle. This operation is similar to ligation of padlock probes described by Nilsson et al., Science, 265:2085-2088 (1994). The ligation operation allows subsequent amplification to be dependent on the presence of a target sequence. Suitable ligases for the ligation operation are described above. Ligation conditions are generally known. Most ligases require Mg⁺⁺. There are two main types of ligases, those that are ATP-dependent and those that are NAD-dependent. ATP or NAD, depending on the type of ligase, should be present during ligation.

The target sequence for an open circle probe can be any nucleic acid or other compound to which the target probe portions of the open circle probe can hybridize in the proper alignment. Target sequences can be found in any nucleic acid molecule from any nucleic acid sample. Thus, target sequences can be in nucleic acids in cell or tissue samples, reactions, and assays. Target sequences can also be artificial nucleic acids (or other compounds to which the target probe portions of the open circle probe can hybridize in the proper alignment). For example, nucleic acid tags can be associated with various of the disclosed compounds to be detected using open circle probes. Thus, a reporter binding agent can contain a target sequence to which an open circle probe can hybridize. In these cases, the target sequence provides a link between the target molecule being detected and the amplification of signal mediated by the open circle probe. When matched open circle probe sets are used, the target sequences will be related based on the relationship of the open circle probes in the set.

When RNA is to be detected, it is preferred that a reverse transcription operation be performed to make a DNA target sequence. Alternatively, an RNA target sequence can be detected directly by using a ligase that can perform ligation on a DNA:RNA hybrid substrate. A preferred ligase for this is T4 DNA ligase.

E. Use of Reporter Binding Agents

A useful form of the disclosed method uses reporter binding agents having amplification target circles or target sequences as the oligonucleotide portion. The amplification target circle can be amplified as described herein. Alternatively, the oligonucleotide portion of the reporter binding agent serves as a target sequence. The affinity portion of the reporter binding agent is a specific binding molecule specific for a target molecule of interest, such as proteins or peptides. The reporter binding agent is associated with the target molecule and detection of this interaction is mediated by rolling circle amplification. Unbound reporter binding agents can be removed by washing. Once the reporter binding agent is associated with a target molecule, the associated amplification target circle can be amplified to detect the target molecule. Alternatively, a open circle probe can be hybridized to the target sequence of the reporter binding agent, ligated, and amplified. The resulting TS-DNA is associated with the ligated open circle probe, thus associating the TS-DNA to the site of the target molecule.

Reporter binding agents are preferably used with a solid-state substrate and in combination with combinatorial multicolor coding. For this purpose, samples to be tested are incorporated into a solid-state sample, as described above. The solid-state substrate is preferably a glass slide and the solid-state sample preferably incorporates up to 256 individual target or assay samples arranged in dots. Multiple solid-state samples can be used to either test more individual samples, or to increase the number of distinct target sequences to be detected. In the later case, each solid-state sample has an identical set of samples dots, and the assay will be carried out using a different set of reporter binding agents and open circle probes, collectively referred to as a probe set, for each solid-state sample. This allows a large number of individuals and target sequences to be assayed in a single assay. By using up to six different labels, combinatorial multicolor coding allows up to 63 distinct targets to be detected on a single solid-state sample. When using multiple solid-state substrates and performing RCA with a different set of reporter binding agents and amplification target circles or open circle probes for each solid-state substrate, the same labels can be used with each solid-state sample (although differences between ATCs or OCPs in each set may require the use of different detection probes). For example, 10 replica slides, each with 256 target sample dots, can be subjected to RCA using 10 different sets of reporter binding agents and amplification target circles or open circle probes, where each set is designed for combinatorial multicolor coding of 63 targets. This results in an assay for detection of 630 different target molecules.

After rolling circle amplification, a cocktail of detection probes can be added, where the cocktail contains color combinations that are specific for each ATC or OCP. The design and combination of such detection probes for use in combinatorial multicolor coding is described elsewhere herein. The labels for combinatorial multicolor detection can be used in the manner of fluorescent change probes. It is preferred that the ATCs or OCPs be designed with combinatorially coded detection tags to allow use of a single set of singly labeled detection probes. It is also preferred that collapsing detection probes be used.

F. Gap-Filling Ligation

The gap space formed by an OCP hybridized to a target sequence is normally occupied by one or more gap oligonucleotides as described above. Such a gap space may also be filled in by a gap-filling DNA polymerase during the ligation operation. As an alternative, the gap space can be partially bridged by one or more gap oligonucleotides, with the remainder of the gap filled using DNA polymerase. This modified ligation operation is referred to herein as gap-filling ligation and is a preferred form of the ligation operation. The principles and procedure for gap-filling ligation are generally analogous to the filling and ligation performed in gap LCR (Wiedmann et al., PCR Methods and Applications (Cold Spring Harbor Laboratory Press, Cold Spring Harbor Laboratory, NY, 1994) pages S51-S64; Abravaya et al., Nucleic Acids Res., 23(4):675-682 (1995); European Patent Application EP0439182 (1991)). In the case of LM-RCA, the gap-filling ligation operation is substituted for the normal ligation operation. Gap-filling ligation provides a means for discriminating between closely related target sequences. Gap-filling ligation can be accomplished by using a different DNA polymerase, referred to herein as a gap-filling DNA polymerase. Suitable gap-filling DNA polymerases are described above. Alternatively, DNA polymerases in general can be used to fill the gap when a stop base is used. The use of stop bases in the gap-filling operation of LCR is described in European Patent Application EP0439182. The principles of the design of gaps and the ends of flanking probes to be joined, as described in EP0439182, is generally applicable to the design of the gap spaces and the ends of target probe portions described herein. Gap-filling ligation is further described in U.S. Pat. No. 6,143,495.

G. Discrimination Between Closely Related Target Sequences

Open circle probes, gap oligonucleotides, and gap spaces can be designed to discriminate closely related target sequences, such as genetic alleles. Where closely related target sequences differ at a single nucleotide, it is preferred that open circle probes be designed with the complement of this nucleotide occurring at one end of the open circle probe, or at one of the ends of the gap oligonucleotide(s). Where gap-filling ligation is used, it is preferred that the distinguishing nucleotide appear opposite the gap space. This allows incorporation of alternative (that is, allelic) sequence into the ligated OCP without the need for alternative gap oligonucleotides. Where gap-filling ligation is used with a gap oligonucleotide(s) that partially fills the gap, it is preferred that the distinguishing nucleotide appear opposite the portion of gap space not filled by a gap oligonucleotide. Ligation of gap oligonucleotides with a mismatch at either terminus is extremely unlikely because of the combined effects of hybrid instability and enzyme discrimination. When the TS-DNA is generated, it will carry a copy of the gap oligonucleotide sequence that led to a correct ligation. Gap oligonucleotides may give even greater discrimination between related target sequences in certain circumstances, such as those involving wobble base pairing of alleles. Features of open circle probes and gap oligonucleotides that increase the target-dependency of the ligation operation are generally analogous to such features developed for use with the ligation chain reaction. These features can be incorporated into open circle probes and gap oligonucleotides for use in LM-RCA. In particular, European Patent Application EP0439182 describes several features for enhancing target-dependency in LCR that can be adapted for use in LM-RCA. The use of stop bases in the gap space, as described in European Patent Application EP0439182, is a preferred mode of enhancing the target discrimination of a gap-filling ligation operation.

A preferred form of target sequence discrimination can be accomplished by employing two types of open circle probes. In one embodiment, a single gap oligonucleotide is used which is the same for both target sequences, that is, the gap oligonucleotide is complementary to both target sequences. In a preferred embodiment, a gap-filling ligation operation can be used (Example 3 in U.S. Pat. No. 6,143,495). Target sequence discrimination would occur by virtue of mutually exclusive ligation events, or extension-ligation events, for which only one of the two open-circle probes is competent. Preferably, the discriminator nucleotide would be located at the penultimate nucleotide from the 3′ end of each of the open circle probes. The two open circle probes would also contain two different detection tags designed to bind alternative detection probes and/or address probes. Each of the two detection probes would have a different detection label. Both open circle probes would have the same primer complement portion. Thus, both ligated open circle probes can be amplified using a single primer. Upon array hybridization, each detection probe would produce a unique signal, for example, two alternative fluorescence colors, corresponding to the alternative target sequences.

These technique for target sequence discrimination are especially useful within matched open circle probe sets.

H. Transcription

Once TS-DNA is generated using RCA, further amplification can be accomplished by transcribing the TS-DNA from promoters embedded in the TS-DNA. This combined process, referred to as rolling circle replication with transcription (RCT) requires that the amplification target circle from which the TS-DNA is made have a promoter portion in its spacer region. The promoter portion is then amplified along with the rest of the amplification target circle resulting in a promoter embedded in each tandem repeat of the TS-DNA. Because transcription, like rolling circle amplification, is a process that can go on continuously (with re-initiation), multiple transcripts can be produced from each of the multiple promoters present in the TS-DNA. RCT effectively adds another level of amplification of amplification target circles. RCT is further described in U.S. Pat. No. 6,143,495. Amplification target circles can also be directly transcribed (that is, not in conjunction with rolling circle amplification). The amplified product will be RNA. Transcription of amplification target circles can produce a long tandem repeat transcript if the amplification target circle does not have a transcription termination sequence.

The transcripts generated in RCT or direct transcription can be labeled and/or detected using the same labels, labeling methods, and detection methods described for use with TS-DNA. Most of these labels and methods are adaptable for use with nucleic acids in general. A useful method of labeling RCT transcripts is by direct labeling of the transcripts by incorporation of labeled nucleotides, most preferably biotinylated nucleotides, during transcription. RCT transcripts can also be detected in real-time, using, for example, fluorescent change probes.

I. Nucleic Acid Library Analysis

The disclosed method can be used to produce replicated strands that serve as a nucleic acid library of a nucleic acid sample. Such a nucleic acid library can be used for any purpose, including, for example, detection of sequences, production of probes, production of nucleic acid arrays or chips, and comparison with nucleic acids in other nucleic acid libraries. Similarly prepared nucleic acid libraries of other nucleic acid samples to allow convenient detection of differences between the samples. The nucleic acid libraries can be used both for detection of related nucleic acid samples and comparison of nucleic acid samples. For example, the presence or identity of specific organisms can be detected by producing a nucleic acid library of the test organism and comparing the resulting nucleic acid library with reference nucleic acid libraries prepared from known organisms. Changes and differences in gene expression patterns can also be detected by preparing nucleic acid libraries of mRNA from different cell samples and comparing the nucleic acid libraries. The replicated strands can also be used to produce a set of probes or primers that is specific for the source of a nucleic acid sample. The replicated strands can also be used as a fingerprint of nucleic acid sequences present in a sample. Nucleic acid libraries can be made up of, or derived from, the mRNA of a sample such that the entire relevant mRNA content of the sample is substantially represented.

Nucleic acid libraries can be stored or archived for later use. For example, replicated strands produced in the disclosed method can be physically stored, either in solution, frozen, or attached or adhered to a solid-state substrate such as an array. Storage in an array is useful for providing an archived probe set derived from the nucleic acids in any sample of interest. As another example, informational content of, or derived from, nucleic acid fingerprints can also be stored. Such information can be stored, for example, in or as computer readable media. Examples of informational content of nucleic acid libraries include nucleic acid sequence information (complete or partial); differential nucleic acid sequence information such as sequences present in one sample but not another; hybridization patterns of replicated strands to, for example, nucleic acid arrays, sets, chips, or other replicated strands. Numerous other data that is or can be derived from nucleic acid libraries and replicated strands produced in the disclosed method can also be collected, used, saved, stored, and/or archived.

Nucleic acid libraries can also contain or be made up of other information derived from the information generated in the disclosed method, and can be combined with information obtained or generated from any other source. The informational nature of nucleic acid libraries produced using the disclosed method lends itself to combination and/or analysis using known bioinformatics systems and methods.

Nucleic acid libraries of nucleic acid samples can be compared to a similar nucleic acid library derived from any other sample to detect similarities and differences in the samples (which is indicative of similarities and differences in the nucleic acids in the samples). For example, a nucleic acid library of a first nucleic acid sample can be compared to a nucleic acid library of a sample from the same type of organism as the first nucleic acid sample, a sample from the same type of tissue as the first nucleic acid sample, a sample from the same organism as the first nucleic acid sample, a sample obtained from the same source but at time different from that of the first nucleic acid sample, a sample from an organism different from that of the first nucleic acid sample, a sample from a type of tissue different from that of the first nucleic acid sample, a sample from a strain of organism different from that of the first nucleic acid sample, a sample from a species of organism different from that of the first nucleic acid sample, or a sample from a type of organism different from that of the first nucleic acid sample.

The same type of tissue is tissue of the same type such as liver tissue, muscle tissue, or skin (which may be from the same or a different organism or type of organism). The same organism refers to the same individual, animal, or cell. For example, two samples taken from a patient are from the same organism. The same source is similar but broader, referring to samples from, for example, the same organism, the same tissue from the same organism, the same DNA molecule, or the same DNA library. Samples from the same source that are to be compared can be collected at different times (thus allowing for potential changes over time to be detected). This is especially useful when the effect of a treatment or change in condition is to be assessed. Samples from the same source that have undergone different treatments can also be collected and compared using the disclosed method. A different organism refers to a different individual organism, such as a different patient, a different individual animal. Different organism includes a different organism of the same type or organisms of different types. A different type of organism refers to organisms of different types such as a dog and cat, a human and a mouse, or E. coli and Salmonella. A different type of tissue refers to tissues of different types such as liver and kidney, or skin and brain. A different strain or species of organism refers to organisms differing in their species or strain designation as those terms are understood in the art.

J. Specific Embodiments

In some forms, the disclosed method involves incubating a mixture comprising an amplification target circle, one or more rolling circle replication primers, and one or more fluorescent change probes, under conditions that promote rolling circle replication of the amplification target circle, wherein the rolling circle replication is primed from a plurality of locations on the amplification target circle, wherein the rolling circle replication results in formation of tandem sequence DNA, and detecting, during the incubation, fluorescent change probes interacting with the tandem sequence DNA.

In some forms, the disclosed method involves incubating a mixture comprising an amplification target circle and one or more rolling circle replication primers under conditions that promote rolling circle replication of the amplification target circle, wherein one or more of the rolling circle replication primers comprise a fluorescent change primer, wherein the rolling circle replication is primed from a plurality of locations on the amplification target circle, wherein the rolling circle replication results in formation of tandem sequence DNA, and detecting, during the incubation, fluorescent change primers incorporated into the tandem sequence DNA.

In some forms, the disclosed method involves incubating a mixture comprising an amplification target circle, one or more rolling circle replication primers, and one or more DNA strand displacement primers under conditions that promote rolling circle replication of the amplification target circle, wherein one or more of the DNA strand displacement primers comprise a fluorescent change primer, wherein the rolling circle replication is primed from a plurality of locations on the amplification target circle, wherein the rolling circle replication results in formation of tandem sequence DNA, and detecting, during the incubation, fluorescent change primers incorporated into the tandem sequence DNA.

In some forms, the disclosed method involves incubating a mixture comprising an amplification target circle, one or more rolling circle replication primers, and one or more fluorescent change probes, under conditions that promote rolling circle replication of the amplification target circle, wherein one or more of the rolling circle replication primers comprise a fluorescent change primer, wherein the rolling circle replication is primed from a plurality of locations on the amplification target circle, wherein the rolling circle replication results in formation of tandem sequence DNA, and detecting, during the incubation, fluorescent change probes interacting with the tandem sequence DNA, fluorescent change primers incorporated into the tandem sequence DNA, or both.

The mixture can contain a plurality of rolling circle replication primers. The mixture can contain one rolling circle replication primer. Detection of fluorescent change probes interacting with the tandem sequence DNA can comprise measuring fluorescence from the fluorescent change probes continuously during the incubation. Detection of fluorescent change probes interacting with the tandem sequence DNA can also comprise measuring the rate of increase in fluorescence from the fluorescent change probes, wherein the rate of increase in fluorescence from the fluorescent change probes indicates the rate of amplification of the amplification target circle, wherein the rate of amplification of the amplification target circle indicates the amount of the amplification target circle present in the mixture. The amplification target circle can be derived from a nucleic acid molecule in a nucleic acid sample, wherein the amount of the amplification target circle present in the mixture indicates the amount of the nucleic acid molecule from which the amplification target circle is derived that is present in the nucleic acid sample. The amplification target circle can comprise a single stranded bacteriophage DNA, a double stranded DNA plasmid or other vector, a bacterial artificial chromosome vector, a yeast artificial chromosome vector, or a clone derived from such a vector. The amplification target circle can be a sub-chromosomal fragment. The sub-chromosomal fragment can be generated by restriction digestion chromosomal DNA and circularization of a chromosomal fragment. The amplification target circle can comprise the nucleic acid molecule in the nucleic acid sample. The amplification target circle can be a bacterial chromosome. The nucleic acid molecule can be human DNA, yeast DNA, mitochondrial DNA, mRNA, cDNA, genomic DNA, viral DNA, viral RNA, bacteriophage DNA, bacteriophage RNA, or precursor RNA.

The amplification target circle can be derived from a nucleic acid molecule, wherein the nucleic acid molecule is human DNA, yeast DNA, mitochondrial DNA, mRNA, cDNA, genomic DNA, viral DNA, viral RNA, bacteriophage DNA, bacteriophage RNA, or precursor RNA. Detection of fluorescent change probes interacting with the tandem sequence DNA can comprise measuring fluorescence from the fluorescent change probes a plurality of times during the incubation. The rolling circle replication primers each can comprise a complementary portion, wherein the amplification target circle can comprise a plurality of primer complement portions, wherein the complementary portion of the rolling circle replication primers can be complementary to one or more of the primer complement portions of the amplification target circle.

The rolling circle replication primers can be random primers. The random primers can comprise unmodified deoxyribonucleotides, unmodified ribonucleotides, modified deoxyribonucleotides, modified ribonucleotides, nucleotide analogs, one or a combination of oligonucleotide analogs, or a combination thereof. The random primers can be chimeric. The rolling circle replication primers can comprise unmodified deoxyribonucleotides, unmodified ribonucleotides, modified deoxyribonucleotides, modified ribonucleotides, nucleotide analogs, one or a combination of oligonucleotide analogs, or a combination thereof. The rolling circle replication primers comprise a mixture of random and specific primers.

The rolling circle replication primers can be within the range of 2 to 50 nucleotides in length. The rolling circle replication primers can be within the range of 2 to 35 nucleotides in length. The rolling circle replication primers can be within the range of 2 to 10 nucleotides in length. At least one of the rolling circle replication primers can be a hexamer. A hexamer is 6 nucleotides in length. At least one of the rolling circle replication primers can be an octamer. An octamer is 8 nucleotides in length. At least one of the rolling circle replication primers can comprise a non-complementary portion, wherein the non-complementary portion need not be complementary to the amplification target circle, wherein the non-complementary portion can be at the 5′ end of the rolling circle replication primer. The amplification target circle can be a single stranded DNA circle. The amplification target circle can be a duplex DNA circle having at least one nick. The amplification target circle can be a duplex DNA circle having no nicks. The method also can include a denaturation step to separate the two strands of the duplex DNA circle.

The amplification target circle can be a supercoiled duplex DNA circle. The amplification target circle can be derived from a nucleic acid sample, wherein the nucleic acid sample is derived from a biological sample. The amplification target circle can be derived from a nucleic acid molecule, wherein the nucleic acid molecule is derived from a biological sample. The biological sample can comprise a bacterial colony, a bacterial cell, a bacteriophage plaque, a bacteriophage, a virus plaque, a virus, a yeast colony, a yeast cell, a baculovirus plaque, a baculovirus, a biological agent, an infectious biological agent, a biological threat agent, a eukaryotic cell culture, a eukaryotic cell, a culture of transiently transfected eukaryotic cells, or a transiently transfected eukaryotic cell. The biological sample can comprise a blood sample, a urine sample, a semen sample, a lymphatic fluid sample, a cerebrospinal fluid sample, a plasma sample, a serum sample, a pus sample, an amniotic fluid sample, a bodily fluid sample, a stool sample, a biopsy sample, a needle aspiration biopsy sample, a swab sample, a mouthwash sample, a cancer sample, a tumor sample, a tissue sample, a cell sample, a cell lysate sample, a crude cell lysate sample, a forensic sample, an environmental sample, an archeological sample, an infection sample, a nosocomial infection sample, a community-acquired infection sample, a biological threat sample, a production sample, a drug preparation sample, a biological molecule production sample, a protein preparation sample, a lipid preparation sample, a carbohydrate preparation sample, or a combination.

The nucleic acid molecule can be human DNA, yeast DNA, mitochondrial DNA, mRNA, cDNA, genomic DNA, viral DNA, viral RNA, bacteriophage DNA, bacteriophage RNA, or precursor RNA. The biological sample can be lysed. Lysis can be achieved by treatment of the biological sample with heat, an enzyme, an organic solvent, or a combination of these. Lysis can be achieved by treatment of the biological sample with an enzyme, wherein the enzyme is lysozyme, glucylase, xymolyase, or a combination of these.

The amplification target circle can be a single stranded RNA circle. The amplification target circle can comprise no more than about 10,000 nucleotides. The amplification target circle can comprise more than 10,000 nucleotides. The amplification target circle can comprises no more than about 1,000 nucleotides. The amplification target circle can comprise no more than about 100 nucleotides. The amplification target circle can comprise a single stranded bacteriophage DNA, a double stranded DNA plasmid or vector, a bacterial artificial chromosome vector, a yeast artificial chromosome vector, or a clone derived from such a vector. The amplification target circle can comprise a nucleic acid molecule in a nucleic acid sample. The amplification target circle can be of unknown sequence composition. The fluorescent change probes each can comprise a complementary portion, wherein the amplification target circle can comprise at least one detection tag portion, wherein the complementary portion of the fluorescent change probes can match the sequence of at least one of the detection tag portions of the amplification target circle.

The mixture can comprise a plurality of amplification target circles. The fluorescent change probes each can comprise a complementary portion, wherein the amplification target circles each can comprise at least one detection tag portion, wherein the complementary portion of each of the fluorescent change probes matches the sequence of one or more of the detection tag portions of the amplification target circles. The mixture can comprise a plurality of fluorescent change probes, wherein the complementary portion of each fluorescent change probe matches the sequence of one or more of the detection tag portions of a different one of the amplification target circles. The mixture can comprise a plurality of fluorescent change probes, wherein the complementary portion of each fluorescent change probe matches the sequence of one or more of the detection tag portions of one or more of the amplification target circles. The mixture can comprises a plurality of fluorescent change probes, wherein the complementary portion of each fluorescent change probe matches the sequence of one of the detection tag portions of a different one of the amplification target circles. The mixture can comprise a plurality of fluorescent change probes, wherein the complementary portion of each fluorescent change probe matches the sequence of a plurality of the detection tag portions of a different one of the amplification target circles. The mixture can comprise a plurality of fluorescent change probes, wherein the complementary portion of each fluorescent change probe matches the sequence of a plurality of the detection tag portions of one of the amplification target circles. The mixture can comprise a plurality of fluorescent change probes, wherein the complementary portion of each fluorescent change probe matches the sequence of a plurality of the detection tag portions of a plurality of the amplification target circles. The mixture can comprise a plurality of fluorescent change probes, wherein the complementary portion of each fluorescent change probe matches the sequence of one of the detection tag portions of a plurality of the amplification target circles. The mixture can comprise a plurality of fluorescent change probes, wherein the complementary portion of each fluorescent change probe matches the sequence of one of the detection tag portions of one of the amplification target circles.

Detection of fluorescent change probes interacting with the tandem sequence DNA can comprise measuring fluorescence from the fluorescent change probes continuously during the incubation. The amplification target circle, the detection tag portion of which matches the sequence of the complementary portion of a fluorescent change probe, can correspond to the fluorescent change probe, wherein detection of fluorescent change probes interacting with the tandem sequence DNA can further comprises measuring the rate of increase in fluorescence from one of the fluorescent change probes, wherein the rate of increase in fluorescence from the fluorescent change probe indicates the rate of amplification of the amplification target circle corresponding to the fluorescent change probe, wherein the rate of amplification of the amplification target circle indicates the amount of the amplification target circle present in the mixture. The amplification target circle, the detection tag portion of which matches the sequence of the complementary portion of a fluorescent change probe, can correspond to that fluorescent change probe, wherein detection of fluorescent change probes interacting with the tandem sequence DNA can further comprise measuring the rate of increase in fluorescence from the fluorescent change probes, wherein the rate of increase in fluorescence from each of the fluorescent change probes indicates the rate of amplification of the amplification target circle corresponding to the fluorescent change probe, wherein the rate of amplification of the amplification target circle indicates the amount of the amplification target circle present in the mixture. The amplification target circles can be derived from nucleic acid molecules, wherein each amplification target circle can be derived from a different nucleic acid molecule. Each nucleic acid molecule can be derived from a different nucleic acid sample, wherein the amount of each amplification target circle present in the mixture indicates the amount of the nucleic acid molecule from which the amplification target circle is derived that is present in the nucleic acid sample from which the nucleic acid molecule is derived.

Replication of each amplification target circle can result in formation of different tandem sequence DNAs. The fluorescent change probes each can comprise a complementary portion, wherein the tandem sequence DNAs each can comprise different probe complement portions, wherein the complementary portion of each of the fluorescent change probes can be complementary to the sequence of a different one of the probe complement portions. At least one amplification target circle can be a plasmid, wherein at least one amplification target circle can be a bacterial chromosome. At least one of the amplification target circles can be eukaryotic chromosomal DNA. The eukaryotic chromosomal DNA can be human chromosomal DNA. The detection can result in detection of the genotype of one or more of the amplification target circles and antibiotic resistance phenotype of one or more of the amplification target circles.

One or more of the fluorescent change probes can be hairpin quenched probes, cleavage quenched probes, cleavage activated probes, fluorescent activated probes, or a combination. The fluorescent change primer can be a hairpin quenched primer.

The mixture can further comprise one or more DNA strand displacement primers, wherein one or more of the DNA strand displacement primers can comprise a fluorescent change primer. One or more of the fluorescent change probes can be hairpin quenched probes, cleavage quenched probes, cleavage activated probes, fluorescent activated probes, or a combination.

### Illustration

A. Amplification of Bacterial Plasmid Using Multiply-Primed Rolling Circle Amplification and Fluorescent Change Probes

Multiply-primed rolling circle amplification of the bacterial plasmids pUC19 and pBR322 can be performed using exonuclease-resistant random hexamers as the rolling circle replication primers, four molecular beacon probes (each complementary to a different sequence in the ampicillin resistance gene in the plasmids) as fluorescent change probes and +29 DNA polymerase as the DNA polymerase.

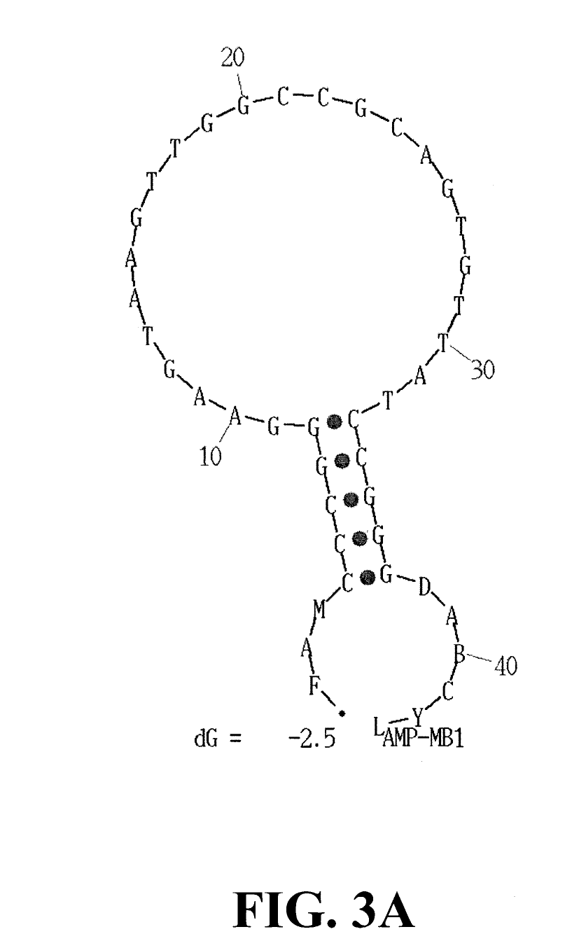

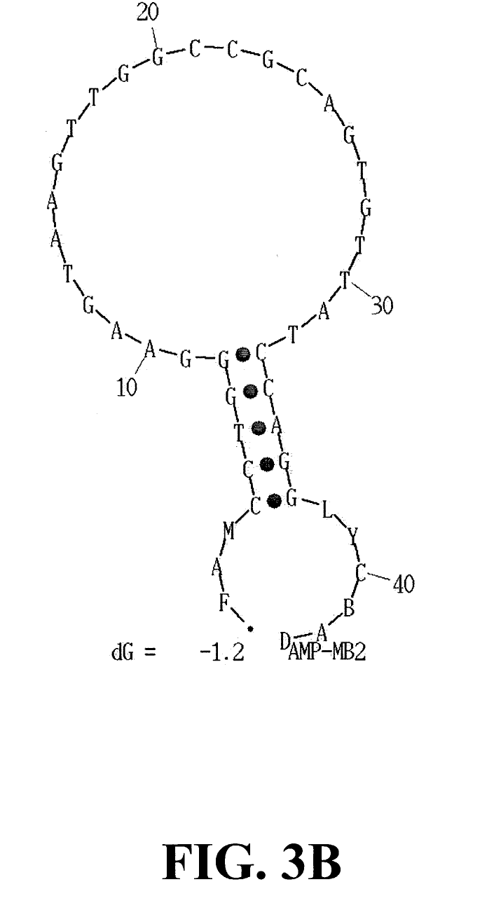

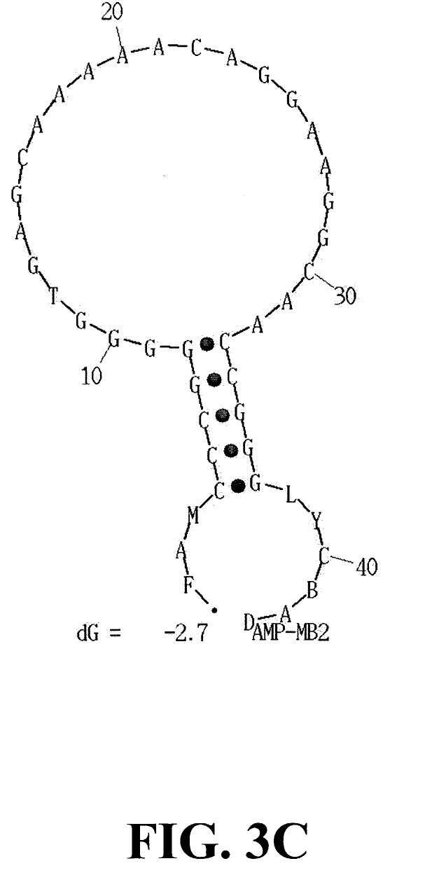

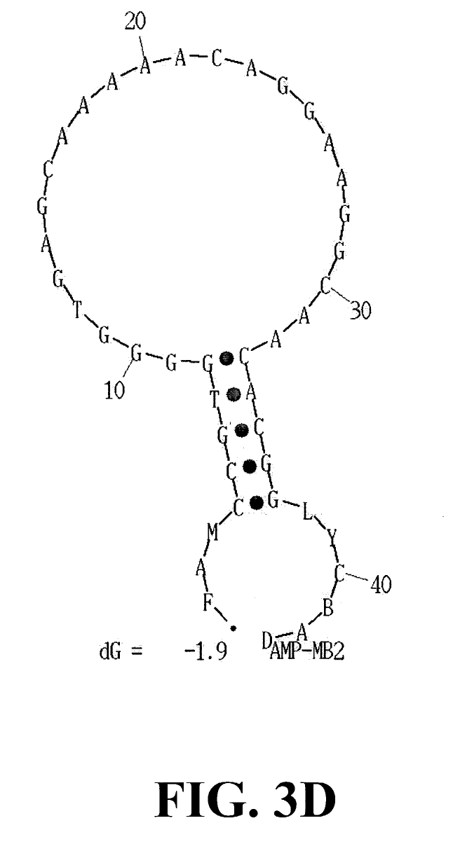

The fluorescent change probes can be AMP-MB1 (FIG. 3A), AMP-MB2 (FIG. 3B), AMP-MB3 (FIG. 3C) and AMP-MB4 (FIG. 3D). AMP-MB1 has a Tm 58.7° C., binds to a sequence in the ampicillin coding region between Pvu I and Sca I site. This is a DNA probe. Sequence: 5′-FAM-cccgg GAA GTA AGT TGG CCG CAG TGT TAT ccggg-DABCYL-3′ (SEQ ID NO:1). Tm for these fluorescent change probes is determined based on a DNA thermodynamics analysis. The 2′-O-Methyl RNA backbone fluorescent change probes will have 5-7° C. higher Tm compared to DNA fluorescent change probes.

AMP-MB2 has a Tm 47.7° C., binds to sequence in the ampicillin coding region between Pvu I and Sca I site. This probe can be either DNA or 2′-O-Methyl RNA backbone. Sequence: 5′-FAM-cctgg GAA GTA AGT TGG CCG CAG TGT TAT ccagg-LYCBAD-3′ (SEQ ID NO:2).

AMP-MB3 has a Tm 60.3° C., binds to sequence in the ampicillin coding region between Eco57 I and Ssp I site. This is a DNA probe. Sequence: 5′-FAM-cccgg GGG TGA GCA AAA ACA GGA AGG CAA ccggg-LYCBAD-3′ (SEQ ID NO:3).

AMP-MB4 has a Tm 52.7° C., binds to sequence in the ampicillin coding region between Eco57 I and Ssp I site. This probe can be either DNA or 2′-O-Methyl RNA backbone. Sequence: 5′-FAM-ccgtg GGG TGA GCA AAA ACA GGA AGG CAA cacgg-LYCBAD-3′ (SEQ ID NO:4).

The fluorescence from the probes can be monitored during the RCA reaction to achieve real-time detection.

### EXAMPLE

A. Example: Real-time detection of multiply-primed rolling circle amplification of plasmid DNA.

demonstrates an embodiment of the disclosed method involving multiply-primed rolling circle amplification and real-time detection of amplification using fluorescent change probes. Nuclease-resistant random hexamer primers were used for multiply-primed RCA, and molecular beacon probes were used as fluorescent change probes for real-time detection. RCA reactions contained 50 mM Tris-HCl pH 7.5, 10 mM MgCl₂, 20 mM ammonium sulfate, 5% glycerol, 200 μg/ml bovine serum albumin, 1 mM each dNTP, 0.02 units yeast pyrophosphatase, 50 μM random hexamer primer, 1 μM molecular beacon, and 0.3 units φ29 DNA polymerase in a total reaction volume of 30 μl. Different reactions included different amounts (0 to 1×10⁸ copies) of the plasmid pUC19. The reactions were incubated at 30° C. for 240 minutes. Fluorescence was monitored during the reaction.

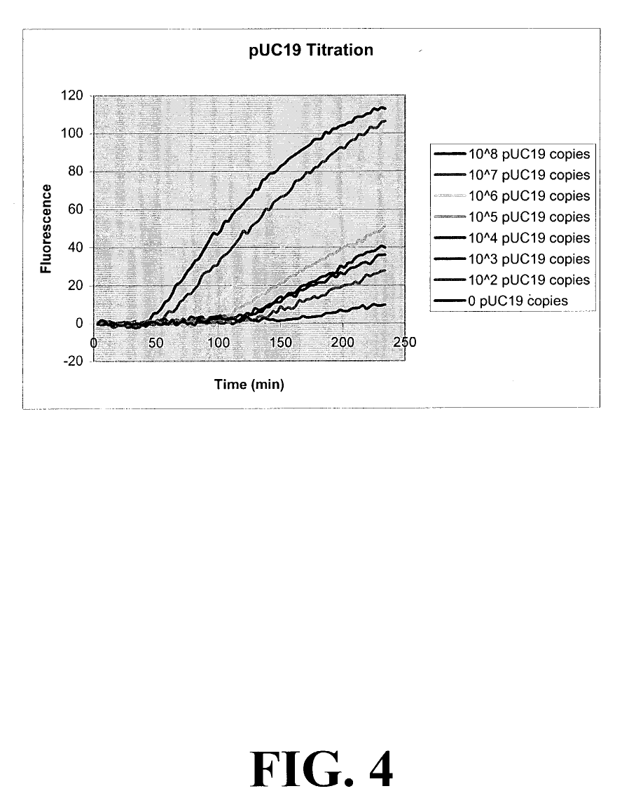

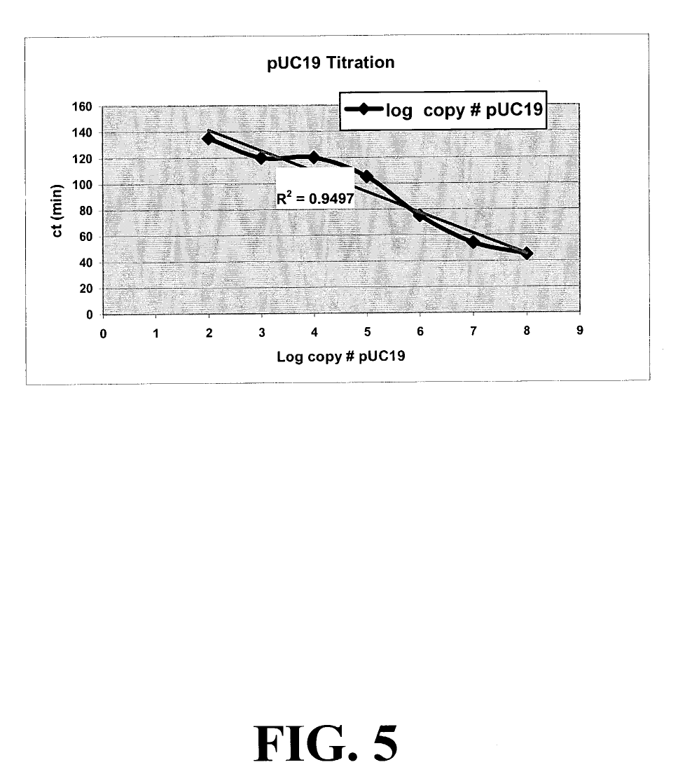

The results are shown in FIG. 4. As can be seen, fluorescence over background appears earlier in the reaction, and reaches a higher level, when more template (that is, pUC19) is used. FIG. 5 plots the log of the number of copies of the template used versus the time at which fluorescence over background is first detected. As can be seen, there is a linear relationship between the amount of template present and the time at which fluorescence over background first appears.

Similar reactions were performed using cell lysates as a source of template rather than purified pUC19. Reactions were performed using cell lysate from E. coli harboring pUC19, cell lysate from E. coli harboring pNEB, cell lysate from E. coli harboring pBR322, cell lysate from E. coli harboring pUC19-HCV, and a control cell lysate from E. coli not harboring a plasmid. The results are shown in FIG. 6. As can be seen, cell lysates can be used as a source of template. Further, the reaction does not produce a detectable signal when an appropriate circular template is not present (see E. coli control). This indicates that the reaction can be both sensitive and specific.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a ”, “an”, and “the” include plural reference unless the context clearly dictates otherwise. Thus, for example, reference to “a primer” includes a plurality of such primers, reference to “the primer” is a reference to one or more primers and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are specifically incorporated by reference. Nothing herein is to be construed as an admission that the present invention is not entitled to antedate such disclosure by virtue of prior invention.

Those skilled in the art will recognize, or be able to ascertain using no more than routine experimentation, many equivalents to the specific embodiments of the method and compositions described herein. Such equivalents are intended to be encompassed by the following claims.

We claim:

1. A method of detecting amplification products during multiply-primed rolling circle amplification, the method comprising
incubating a mixture comprising an amplification target circle, one or more rolling circle replication primers, and one or more fluorescent change probes, under conditions that promote rolling circle replication of the amplification target circle, wherein the rolling circle replication is primed from a plurality of locations on the amplification target circle, wherein the rolling circle replication results in formation of tandem sequence DNA, and
detecting, during the incubation, fluorescent change probes interacting with the tandem sequence DNA.

2. The method of claim 1 wherein the mixture contains a plurality of rolling circle replication primers.

3. The method of claim 1 wherein the mixture contains one rolling circle replication primer.

4. The method of claim 1 wherein detection of fluorescent change probes interacting with the tandem sequence DNA comprises measuring fluorescence from the fluorescent change probes continuously during the incubation.

5. The method of claim 4 wherein detection of fluorescent change probes interacting with the tandem sequence DNA further comprises measuring the rate of increase in fluorescence from the fluorescent change probes, wherein the rate of increase in fluorescence from the fluorescent change probes indicates the rate of amplification of the amplification target circle, wherein the rate of amplification of the amplification target circle indicates the amount of the amplification target circle present in the mixture.

6. The method of claim 5 wherein the amplification target circle is derived from a nucleic acid molecule in a nucleic acid sample, wherein the amount of the amplification target circle present in the mixture indicates the amount of the nucleic acid molecule from which the amplification target circle is derived that is present in the nucleic acid sample.

7. The method of claim 6 wherein the amplification target circle comprises a single stranded bacteriophage DNA, a double stranded DNA plasmid or other vector, a bacterial artificial chromosome vector, a yeast artificial chromosome vector, or a clone derived from such a vector.

8. The method of claim 7 wherein the amplification target circle is a sub-chromosomal fragment.

9. The method of claim 8 wherein the sub-chromosomal fragment is generated by restriction digestion chromosomal DNA and circularization of a chromosomal fragment.

10. The method of claim 6 wherein the amplification target circle comprises the nucleic acid molecule in the nucleic acid sample.

11. The method of claim 10 wherein the amplification target circle is a bacterial chromosome.

12. The method of claim 6 wherein the nucleic acid molecule is human DNA, yeast DNA, mitochondrial DNA, mRNA, cDNA, genomic DNA, viral DNA, viral RNA, bacteriophage DNA, bacteriophage RNA, or precursor RNA.

13. The method of claim 1 wherein the amplification target circle is derived from a nucleic acid molecule, wherein the nucleic acid molecule is human DNA, yeast DNA, mitochondrial DNA, mRNA, cDNA, genomic DNA, viral DNA, viral RNA, bacteriophage DNA, bacteriophage RNA, or precursor RNA.

14. The method of claim 1 wherein detection of fluorescent change probes interacting with the tandem sequence DNA comprises measuring fluorescence from the fluorescent change probes a plurality of times during the incubation.

15. The method of claim 1 wherein the rolling circle replication primers each comprise a complementary portion, wherein the amplification target circle comprises a plurality of primer complement portions, wherein the complementary portion of the rolling circle replication primers is complementary to one or more of the primer complement portions of the amplification target circle.

16. The method of claim 1 wherein the rolling circle replication primers are random primers.

17. The method of claim 16 wherein the random primers comprise unmodified deoxyribonucleotides, unmodified ribonucleotides, modified deoxyribonucleotides, modified ribonucleotides, nucleotide analogs, one or a combination of oligonucleotide analogs, or a combination thereof.

18. The method of claim 17 wherein the random primers are chimeric.

19. The method of claim 1 wherein the rolling circle replication primers comprise unmodified deoxyribonucleotides, unmodified ribonucleotides, modified deoxyribonucleotides, modified ribonucleotides, nucleotide analogs, one or a combination of oligonucleotide analogs, or a combination thereof.

20. The method of claim 19 wherein the rolling circle replication primers are chimeric.

21. The method of claim 1 wherein the rolling circle replication primers comprise a mixture of random and specific primers.

22. The method of claim 1 wherein the rolling circle replication primers are within the range of 2 to 50 nucleotides in length.

23. The method of claim 1 wherein the rolling circle replication primers are within the range of 2 to 35 nucleotides in length.

24. The method of claim 1 wherein the rolling circle replication primers are within the range of 2 to 10 nucleotides in length.

25. The method of claim 1 wherein at least one of the rolling circle replication primers are hexamers.

26. The method of claim 1 wherein at least one of the rolling circle replication primers are octamers.

27. The method of claim 1 wherein at least one of the rolling circle replication primers comprises a non-complementary portion, wherein the non-complementary portion is not complementary to the amplification target circle, wherein the non-complementary portion is at the 5′ end of the rolling circle replication primer.

28. The method of claim 1 wherein the amplification target circle is a single stranded DNA circle.

29. The method of claim 1 wherein the amplification target circle is a duplex DNA circle having at least one nick.

30. The method of claim 1 wherein the amplification target circle is a duplex DNA circle having no nicks.

31. The method of claim 30 further comprising a denaturation step to separate the two strands of the duplex DNA circle.

32. The method of claim 1 wherein the amplification target circle is a supercoiled duplex DNA circle.

33. The method of claim 1 wherein the amplification target circle is derived from a nucleic acid sample, wherein the nucleic acid sample is derived from a biological sample.

34. The method of claim 33 wherein the biological sample comprises a bacterial colony, a bacterial cell, a bacteriophage plaque, a bacteriophage, a virus plaque, a virus, a yeast colony, a yeast cell, a baculovirus plaque, a baculovirus, a biological agent, an infectious biological agent, a biological threat agent, a eukaryotic cell culture, a eukaryotic cell, a culture of transiently transfected eukaryotic cells, or a transiently transfected eukaryotic cell.

35. The method of claim 33 wherein the biological sample comprises a blood sample, a urine sample, a semen sample, a lymphatic fluid sample, a cerebrospinal fluid sample, a plasma sample, a serum sample, a pus sample, an amniotic fluid sample, a bodily fluid sample, a stool sample, a biopsy sample, a needle aspiration biopsy sample, a swab sample, a mouthwash sample, a cancer sample, a tumor sample, a tissue sample, a cell sample, a cell lysate sample, a crude cell lysate sample, a forensic sample, an environmental sample, an archeological sample, an infection sample, a nosocomial infection sample, a community-acquired infection sample, a biological threat sample, a production sample, a drug preparation sample, a biological molecule production sample, a protein preparation sample, a lipid preparation sample, a carbohydrate preparation sample, or a combination.

36. The method of claim 1 wherein the amplification target circle is derived from a nucleic acid molecule, wherein the nucleic acid molecule is derived from a biological sample.

37. The method of claim 36 wherein the biological sample comprises a blood sample, a urine sample, a semen sample, a lymphatic fluid sample, a cerebrospinal fluid sample, a plasma sample, a serum sample, a pus sample, an amniotic fluid sample, a bodily fluid sample, a stool sample, a biopsy sample, a needle aspiration biopsy sample, a swab sample, a mouthwash sample, a cancer sample, a tumor sample, a tissue sample, a cell sample, a cell lysate sample, a crude cell lysate sample, a forensic sample, an environmental sample, an archeological sample, an infection sample, a nosocomial infection sample, a community-acquired infection sample, a biological threat sample, a production sample, a drug preparation sample, a biological molecule production sample, a protein preparation sample, a lipid preparation sample, a carbohydrate preparation sample, or a combination.

38. The method of claim 37 wherein the biological sample comprises a bacterial colony, a bacterial cell, a bacteriophage plaque, a bacteriophage, a virus plaque, a virus, a yeast colony, a yeast cell, a baculovirus plaque, a baculovirus, a biological agent, an infectious biological agent, a biological threat agent, a eukaryotic cell culture, a eukaryotic cell, a culture of transiently transfected eukaryotic cells, or a transiently transfected eukaryotic cell.

39. The method of claim 38 wherein the nucleic acid molecule is human DNA, yeast DNA, mitochondrial DNA, mRNA, cDNA, genomic DNA, viral DNA, viral RNA, bacteriophage DNA, bacteriophage RNA, or precursor RNA.

40. The method of claim 33 wherein the biological sample is lysed.

41. The method of claim 40 wherein lysis is achieved by treatment of the biological sample with heat, an enzyme, an organic solvent, or a combination of these.

42. The method of claim 41 wherein lysis is achieved by treatment of the biological sample with an enzyme, wherein the enzyme is lysozyme, glucylase, xymolyase, or a combination of these.

43. The method of claim 1 wherein the amplification target circle is a single stranded RNA circle.

44. The method of claim 1 wherein the amplification target circle comprises no more than about 10,000 nucleotides.

45. The method of claim 1 wherein the amplification target circle comprises more than 10,000 nucleotides.

46. The method of claim 1 wherein the amplification target circle comprises no more than about 1,000 nucleotides.

47. The method of claim 1 wherein the amplification target circle comprise no more than about 100 nucleotides.

48. The method of claim 1 wherein the amplification target circle comprises a single stranded bacteriophage DNA, a double stranded DNA plasmid or vector, a bacterial artificial chromosome vector, a yeast artificial chromosome vector, or a clone derived from such a vector.

49. The method of claim 1 wherein the amplification target circle comprises a nucleic acid molecule in a nucleic acid sample.

50. The method of claim 1 wherein the amplification target circle is of unknown sequence composition.

51. The method of claim 1 wherein the fluorescent change probes each comprise a complementary portion, wherein the amplification target circle comprises at least one detection tag portion, wherein the complementary portion of the fluorescent change probes matches the sequence of at least one of the detection tag portions of the amplification target circle.

52. The method of claim 1 wherein the mixture comprises a plurality of amplification target circles.

53. The method of claim 52 wherein the fluorescent change probes each comprise a complementary portion, wherein the amplification target circles each comprise at least one detection tag portion, wherein the complementary portion of each of the fluorescent change probes matches the sequence of one or more of the detection tag portions of the amplification target circles.

54. The method of claim 53 wherein the mixture comprises a plurality of fluorescent change probes, wherein the complementary portion of each fluorescent change probe matches the sequence of one or more of the detection tag portions of a different one of the amplification target circles.

55. The method of claim 53 wherein the mixture comprises a plurality of fluorescent change probes, wherein the complementary portion of each fluorescent change probe matches the sequence of one or more of the detection tag portions of one or more of the amplification target circles.

56. The method of claim 53 wherein the mixture comprises a plurality of fluorescent change probes, wherein the complementary portion of each fluorescent change probe matches the sequence of one of the detection tag portions of a different one of the amplification target circles.

57. The method of claim 53 wherein the mixture comprises a plurality of fluorescent change probes, wherein the complementary portion of each fluorescent change probe matches the sequence of a plurality of the detection tag portions of a different one of the amplification target circles.

58. The method of claim 53 wherein the mixture comprises a plurality of fluorescent change probes, wherein the complementary portion of each fluorescent change probe matches the sequence of a plurality of the detection tag portions of one of the amplification target circles.

59. The method of claim 53 wherein the mixture comprises a plurality of fluorescent change probes, wherein the complementary portion of each fluorescent change probe matches the sequence of a plurality of the detection tag portions of a plurality of the amplification target circles.

60. The method of claim 53 wherein the mixture comprises a plurality of fluorescent change probes, wherein the complementary portion of each fluorescent change probe matches the sequence of one of the detection tag portions of a plurality of the amplification target circles.

61. The method of claim 53 wherein the mixture comprises a plurality of fluorescent change probes, wherein the complementary portion of each fluorescent change probe matches the sequence of one of the detection tag portions of one of the amplification target circles.

62. The method of claim 53 wherein detection of fluorescent change probes interacting with the tandem sequence DNA comprises measuring fluorescence from the fluorescent change probes continuously during the incubation.

63. The method of claim 62 wherein the amplification target circle, the detection tag portion of which matches the sequence of the complementary portion of a fluorescent change probe, corresponds to the fluorescent change probe, wherein detection of fluorescent change probes interacting with the tandem sequence DNA further comprises measuring the rate of increase in fluorescence from one of the fluorescent change probes, wherein the rate of increase in fluorescence from the fluorescent change probe indicates the rate of amplification of the amplification target circle corresponding to the fluorescent change probe, wherein the rate of amplification of the amplification target circle indicates the amount of the amplification target circle present in the mixture.

64. The method of claim 62 wherein the amplification target circle, the detection tag portion of which matches the sequence of the complementary portion of a fluorescent change probe, corresponds to that fluorescent change probe, wherein detection of fluorescent change probes interacting with the tandem sequence DNA further comprises measuring the rate of increase in fluorescence from the fluorescent change probes, wherein the rate of increase in fluorescence from each of the fluorescent change probes indicates the rate of amplification of the amplification target circle corresponding to the fluorescent change probe, wherein the rate of amplification of the amplification target circle indicates the amount of the amplification target circle present in the mixture.

65. The method of claim 64 wherein the amplification target circles are derived from nucleic acid molecules, wherein each amplification target circle is derived from a different nucleic acid molecule.

66. The method of claim 65 wherein each nucleic acid molecule is derived from a different nucleic acid sample, wherein the amount of each amplification target circle present in the mixture indicates the amount of the nucleic acid molecule from which the amplification target circle is derived that is present in the nucleic acid sample from which the nucleic acid molecule is derived.

67. The method of claim 52 wherein replication of each amplification target circle results in formation of different tandem sequence DNAs.

68. The method of claim 67 wherein the fluorescent change probes each comprise a complementary portion, wherein the tandem sequence DNAs each comprise different probe complement portions, wherein the complementary portion of each of the fluorescent change probes is complementary to the sequence of a different one of the probe complement portions.

69. The method of claim 52 wherein at least one amplification target circle is a plasmid, wherein at least one amplification target circle is a bacterial chromosome.

70. The method of claim 69 wherein at least one of the amplification target circles is eukaryotic chromosomal DNA.

71. The method of claim 70 wherein the eukaryotic chromosomal DNA is human chromosomal DNA.

72. The method of claim 69 wherein the detection results in detection of the genotype of one or more of the amplification target circles and antibiotic resistance phenotype of one or more of the amplification target circles.

73. The method of claim 1 wherein one or more of the fluorescent change probes are hairpin quenched probes, cleavage quenched probes, cleavage activated probes, fluorescent activated probes, or a combination.

74. A method of detecting amplification products during multiply-primed rolling circle amplification, the method comprising
incubating a mixture comprising an amplification target circle and one or more rolling circle replication primers under conditions that promote rolling circle replication of the amplification target circle, wherein one or more of the rolling circle replication primers comprise a fluorescent change primer, wherein the rolling circle replication is primed from a plurality of locations on the amplification target circle, wherein the rolling circle replication results in formation of tandem sequence DNA, and
detecting, during the incubation, fluorescent change primers incorporated into the tandem sequence DNA.

75. The method of claim 74 wherein the fluorescent change primer is a hairpin quenched primer.

76. A method of detecting amplification products during multiply-primed rolling circle amplification, the method comprising
incubating a mixture comprising an amplification target circle, one or more rolling circle replication primers, and one or more DNA strand displacement primers under conditions that promote rolling circle replication of the amplification target circle, wherein one or more of the DNA strand displacement primers comprise a fluorescent change primer, wherein the rolling circle replication is primed from a plurality of locations on the amplification target circle, wherein the rolling circle replication results in formation of tandem sequence DNA, and
detecting, during the incubation, fluorescent change primers incorporated into the tandem sequence DNA.

77. The method of claim 76 wherein the fluorescent change primer is a hairpin quenched primer.

78. A method of detecting amplification products during multiply-primed rolling circle amplification, the method comprising
incubating a mixture comprising an amplification target circle, one or more rolling circle replication primers, and one or more fluorescent change probes, under conditions that promote rolling circle replication of the amplification target circle, wherein one or more of the rolling circle replication primers comprise a fluorescent change primer, wherein the rolling circle replication is primed from a plurality of locations on the amplification target circle, wherein the rolling circle replication results in formation of tandem sequence DNA, and
detecting, during the incubation, fluorescent change probes interacting with the tandem sequence DNA, fluorescent change primers incorporated into the tandem sequence DNA, or both.

79. The method of claim 78 wherein the mixture further comprises one or more DNA strand displacement primers, wherein one or more of the DNA strand displacement primers comprise a fluorescent change primer.

80. The method of claim 78 wherein one or more of the fluorescent change probes are hairpin quenched probes, cleavage quenched probes, cleavage activated probes, fluorescent activated probes, or a combination.

81. The method of claim 78 wherein the fluorescent change primer is a hairpin quenched primer.

82. The method of claim 1 wherein one or more of the fluorescent change probes are triplex hairpin quenched probes.

83. The method of claim 1 wherein one or more of the fluorescent change probes are triplex FRET probes.


SEQUENCE LISTING (4 sequences)

SEQ ID NO: 1 - DNA, 34 bp, Artificial Sequence
  Description of Artificial Sequence /Note=Synthetic Construct
cccgggaagtaagttggccgcagtgttatccggg

SEQ ID NO: 2 - DNA, 34 bp, Artificial Sequence
  Description of Artificial Sequence /Note=Synthetic Construct
cctgggaagtaagttggccgcagtgttatccagg

SEQ ID NO: 3 - DNA, 34 bp, Artificial Sequence
  Description of Artificial Sequence /Note=Synthetic Construct
cccgggggtgagcaaaaacaggaaggcaaccggg

SEQ ID NO: 4 - DNA, 34 bp, Artificial Sequence
  Description of Artificial Sequence /Note=Synthetic Construct
ccgtggggtgagcaaaaacaggaaggcaacacgg